(19) United States
(12) Patent Application Publication
(10) Pub. No.: US 2004/0082602 A1
(43) Pub. Date: Apr. 29, 2004

(54) Substituted thiophene carboxamide compounds for the treatment of inflammation

(76) Inventors: Timothy J. Hagen, Gurnee, IL (US); Richard M. Weier, Lake Bluff, IL (US); Xiangdong Xu, Gurnee, IL (US); Stephen C. Houdek, Des Plaines, IL (US); Michael Clare, Skokie, IL (US)

Correspondence Address: Pharmacia Corporation, Global Patent Department, 5th Floor, 575 Maryville Centre Drive, St. Louis, MO, 63141, US

(21) Appl. No.: 10/623,228

(22) Filed: Jul. 18, 2003

Related U.S. Application Data
(60) Provisional application No. 60/397,052, filed on Jul. 19, 2002.

(51) Int. Cl. A61K 31/4743; A61K 31/407; A61K 31/381

(52) U.S. Cl. 514/291; 514/411; 514/443; 546/80; 548/430; 549/43

(57) ABSTRACT

The present invention provides substituted thiophene carboxamide compounds having structural Formula I and isomers, tautomers, polymorphs, carriers, prodrugs, and pharmaceutically acceptable salts thereof, compositions comprising such, and methods for treating diseases associated with kinase activity. More specifically, the present invention provides methods of treatment of a variety of diseases associated with IKK2 including the treatment of inflammation, other inflammation-associated disorders, such as, as an analgesic in the treatment of pain and headaches, arthritis, including but not limited to rheumatoid arthritis, asthma, gastrointestinal conditions such as inflammatory bowel disease, vascular diseases, viral infections such as AIDS, and cancer.

The present application claims priority under Title 35, United States Code, §119 to U.S. Provisional application Serial No. 60/397,052, filed Jul. 19, 2002, which is incorporated by reference in its entirety as if written herein.

### FIELD OF THE INVENTION

The present invention in general is in the field of anti-inflammatory pharmaceutical agents and specifically relates to substituted thiophene carboxamide derivatives, compositions comprising such, and methods for treating cancer, inflammation, and inflammation-associated disorders, such as arthritis.

### BACKGROUND OF THE INVENTION

The following description of the background of the invention is provided to aid in the understanding the invention, but is not admitted to be or describe prior art to the invention. NF-κB is a ubiquitous transcription factor that plays a prominent role in the activation of the immune system and in stress responses by regulating the transcription of many early, inducible genes including proinflammatory cytokines, adhesion molecules, growth factors, enzymes, and receptors (Ghosh S., May, M. J., and Kopp. E (1998) Annu. Rev. Immunol. 16, 115-260; Zandi, E., and Karin, M. (1999) Mol. Cell. Biol. 19, 4547-4551; Karin, M. (1999) J. Biol. Chem. 274, 27339-27342). Specificity of gene expression is determined at a cellular level by a diverse array of external stimuli such as bacterial products including LPS, as well as cytokines, most importantly tumor necrosis factor-α (TNFα) and interleukin-β (IL1β). Through the synergistic interaction with other transcription factors, further specificity can be achieved while maintaining enormous potential to coordinately induce a large number of functionally related genes. NF-κB is composed of homo and heterodimers of the Rel protein family and is sequestered in an inactive form in the cytoplasm by members of the IκB family of inhibitory proteins (Ghosh S., May, M. J., and Kopp. E (1998) Annu. Rev. Immunol. 16, 115-260; Zandi, E., and Karin, M. (1999) Mol. Cell. Biol. 19, 4547-4551; Karin, M. (1999) J. Biol. Chem. 274, 27339-27342). IκBs mask the nuclear localization signal on NF-κB, preventing nuclear translocation and hence DNA binding to the promoter regions of responsive genes. Stimulation of cells with an agonist that activates NF-κB leads to a series of biochemical signals, ultimately resulting in the phosphorylation, ubiquitinylation, and degradation of IκBs, thereby releasing NF-κB for nuclear translocation (Ghosh S., May, M. J., and Kopp. E (1998) Annu. Rev. Immunol. 16, 115-260; Zandi, E., and Karin, M. (1999) Mol. Cell. Biol. 19, 4547-4551; Karin, M. (1999) J. Biol. Chem. 274, 27339-27342). Recently, two IκB kinases (IKK1 or IKKα and IKK2 or IKKβ), which phosphorylate IκdBs and thereby initiate their degradation, have been cloned and characterized by a number of laboratories (Ghosh S., May, M. J., and Kopp. E (1998) Annu. Rev. Immunol. 16, 115-260; Zandi, E., and Karin, M. (1999) Mol. Cell. Biol. 19, 4547-4551; Karin, M. (1999) J. Biol. Chem. 274, 27339-27342). The catalytic subunits, IKK1 and IKK2, are similar structurally as well as enzymatically and exist as a heterodimer in a large protein complex referred to as the IKK signalsome (Regnier, C., Song, H., Gao, X., Goeddel, D., Cao, Z. and Rothe, M. (1997) Cell 90, 373-383; DiDonato, J. A., Hayakawa, M., Rothwarf, D. M., Zandi, E. and Karin, M. (1997) Nature 388, 548-554; Mercurio, F., Zhu, H., Murray, B. W., Shevchenko, A., Bennett, B. L., Li, J. W., Young, D. B., Barbosa, M., Mann, M., Manning, A. and Roa, A. (1997) Science 278, 860-866; Zandi, E. Rothwarf, D. M., Delhase, M., Hayadawa, M and Karin, M. (1997) Cell 91, 243-252; Woronicz, J. D., Gao, X., Cao, Z., Rothe, M. And Goeddel, D. V. (1997) Science 278, 866-869). A third protein, NEMO (IKKγ, IKKAP1), is a regulatory adapter protein necessary for IKK activation and kinase activity (Yamaoka, S., Courtois, G., Bessia, C., Whiteside, S. T., Weil, R., Agou, F., Kirk, H. E., Kay, R. J., and Ireal, A. (1998) Cell 93, 1231-1240; Rothwarf, D. M., Zandi, E., Natoli, G., Karin, M. (1998) Nature 395, 297; Mercurio, F., Murray, B. W., Shevchenko, A., Bennet, B. L., Young, D. B., Li, J. W., Pascual, G., Motiwala, A., Zhu, H., Mann, M and Manning, A. M. (1999) Mol. Cell. Biol. 2, 1526-1538). IKK1 and IKK2 are co-expressed in most human adult tissues as well as in different developmental stages of mouse embryos (Regnier, C., Song, H., Gao, X., Goeddel, D., Cao, Z. and Rothe, M. (1997) Cell 90, 373-383; DiDonato, J. A., Hayakawa, M., Rothwarf, D. M., Zandi, E. and Karin, M. (1997) Nature 388, 548-554; Mercurio, F., Zhu, H., Murray, B. W., Shevchenko, A., Bennett, B. L., Li, J. W., Young, D. B., Barbosa, M., Mann, M., Manning, A. and Roa, A. (1997) Science 278, 860-866; Zandi, E. Rothwarf, D. M., Delhase, M., Hayadawa, M and Karin, M. (1997) Cell 91, 243-252; Woronicz, J. D., Gao, X., Cao, Z., Rothe, M. and Goeddel, D. V. (1997) Science 278, 866-869; Hu, M. C. T., and Wang, Y. (1998) Gene 222, 31-40). This kinase complex appears to represent a critical, common denominator in the activation of NF-κB in a number of signal transduction pathways stimulated by a variety of agonists including cytokines, such as TNFβ and IL1β, microbial products such as LPS and viral proteins such as TAX, as well as phorbol esters, oxidizing agents and serine/tyrosine phosphatases (Ghosh S., May, M. J., and Kopp. E (1998) Annu. Rev. Immunol. 16, 115-260; Zandi, E., and Karin, M. (1999) Mol. Cell. Biol. 19, 4547-4551; Karin, M. (1999) J. Biol. Chem. 274, 27339-27342).

IKK1 (also termed IKKα, Regnier, C., Song, H., Gao, X., Goeddel, D., Cao, Z. and Rothe, M. (1997) Cell 90, 373-383; DiDonato, J. A., Hayakawa, M., Rothwarf, D. M., Zandi, E. and Karin, M. (1997) Nature 388, 548-554; Mercurio, F., Zhu, H., Murray, B. W., Shevchenko, A., Bennett, B. L., Li, J. W., Young, D. B., Barbosa, M., Mann, M., Manning, A. And Roa, A. (1997) Science 278, 860-866) was cloned simultaneously by standard biochemical purification of the IcB kinase activity from TNFα stimulated HeLa S3 cells and by its interaction with the MAP3K, NF-κB inducing kinase (NIK), in a yeast two-hybrid screen. IKK1 was identified as the previously cloned serine-threonine kinase, CHUK (Connelly, M. and Marcu, K. (1995) Cell. Mol. Biol. Res. 41, 537-549). IKK1 (also termed IKKα) is an 85 kDa, 745 amino acid protein that contains an N-terminal serine/threonine kinase catalytic domain, a leucine zipper-like amphipathic helix, and a C-terminal helix-loop-helix domain. IKK2 (also termed IKK0) was also cloned by standard biochemical purification, copurifying with IKK1 from TNFα stimulated HeLa S3 cells as well as by being identified in the public database from an EST clone with sequence homology to IKK1 (Mercurio, F., Zhu, H., Murray, B. W., Shevchenko, A., Bennett, B. L., Li, J. W., Young, D. B., Barbosa, M., Mann, M., Manning, A. and Roa, A. (1997) Science 278, 860-866; Zandi, E. Rothwarf, D. M., Delhase, M., Hayadawa, M and Karin, M. (1997) Cell 91, 243-252; Woronicz, J. D., Gao, X., Cao, Z., Rothe, M. And Goeddel, D. V. (1997) Science 278, 866-869). IKK2 is an 87 kDa, 756 amino acid protein with the same over all topology as IKK1 except for the addition of an 11 amino acid extension at the C-terminus. IKK1 and IKK2 are 52% identical overall with 65% identity in the kinase domain and 44% identity in the protein interaction domains in the C-terminus. Data obtained using transient mammalian expression analysis, by in vitro translation experiments and by coexpression in a baculoviral system reveals that IKK1 and IKK2 associate preferentially as a heterodimer through their leucine zipper motifs. Although homodimers have also been described in these systems, the heterodimer is thought to be the physiologic form of the kinase in mammalian cells (Zandi, E. Rothwarf, D. M., Delhase, M., Hayadawa, M and Karin, M. (1997) Cell 91, 243-252; Li, J., Peet, G. W., Pullen, S. S., Schembri-King, J., Warren, T. C., Marcu, K. B., Kehry, M. R., Barton, R. and Jakes, S. (1998) J. Biol. Chem. 273, 30736-30741). Finally, NEMO (also termed IKKγ) contains three α-helical regions including a leucine zipper, interacts preferentially with IKK2 and is required for activation of the heterodimeric kinase complex perhaps by bringing other proteins into the signalsome complex (Yamaoka, S., Courtois, G., Bessia, C., Whiteside, S. T., Weil, R., Agou, F., Kirk, H. E., Kay, R. J., and Ireal, A. (1998) Cell 93, 1231-1240; Rothwarf, D. M., Zandi, E., Natoli, G., Karin, M. (1998) Nature 395, 297; Mercurio, F., Murray, B. W., Shevchenko, A., Bennet, B. L., Young, D. B., Li, J. W., Pascual, G., Motiwala, A., Zhu, H., Mann, M and Manning, A. M. (1999) Mol. Cell. Biol. 2, 1526-1538).

The kinase activities of IKK1 and IKK2 are regulated by phosphorylation and require an intact leucine zipper (LZ) for dimerization as well as an intact helix-loop-helix (HLH) domain, which can exert a positive regulatory effect on kinase activity even when it is expressed in trans with the remainder of the IKK protein (Regnier, C., Song, H., Gao, X., Goeddel, D., Cao, Z. and Rothe, M. (1997) Cell 90, 373-383; DiDonato, J. A., Hayakawa, M., Rothwarf, D. M., Zandi, E. and Karin, M. (1997) Nature 388, 548-554; Mercurio, F., Zhu, H., Murray, B. W., Shevchenko, A., Bennett, B. L., Li, J. W., Young, D. B., Barbosa, M., Mann, M., Manning, A. and Roa, A. (1997) Science 278, 860-866; Zandi, E. Rothwarf, D. M., Delhase, M., Hayadawa, M and Karin, M. (1997) Cell 91, 243-252; Woronicz, J. D., Gao, X., Cao, Z., Rothe, M. and Goeddel, D. V. (1997) Science 278, 866-869; Dehase, M., Hayakawa, M., Chen, Y., and Karin, M. (1999) Science 284, 309-313). Both IKK subunits contain a canonical MAPKK activation loop motif near the N-terminus which is the target for phosphorylation and activation of kinase activity by MAP3Ks such as NIK and MEKK1, although the physiologic regulation by these two upstream kinases awaits further characterization (Zandi, E., and Karin, M. (1999) Mol. Cell. Biol. 19, 4547-4551; Karin, M. (1999) J. Biol. Chem. 274, 27339-27342; Karin, M., and Delhase, M. (1998) Proc. Natl. Acad. Sci. USA 95, 9067-9069). Finally, phosphorylation of serines in the C-terminus of IKK2 results in a decrease in IKK activity and it is postulated to be responsible for the transient kinase activity seen after stimulation of cells with an agonist (Dehase, M., Hayakawa, M., Chen, Y., and Karin, M. (1999) Science 284, 309-313).

IKK2 demonstrates a more potent kinase activity compared to IKK1 using IκBα or IκBβ as a substrate (Mercurio, F., Zhu, H., Murray, B. W., Shevchenko, A., Bennett, B. L., Li, J. W., Young, D. B., Barbosa, M., Mann, M., Manning, A. and Roa, A. (1997) Science 278, 860-866; Zandi, E. Rothwarf, D. M., Delhase, M., Hayadawa, M and Karin, M. (1997) Cell 91, 243-252; Woronicz, J. D., Gao, X., Cao, Z., Rothe, M. and Goeddel, D. V. (1997) Science 278, 866-869; Dehase, M., Hayakawa, M., Chen, Y., and Karin, M. (1999) Science 284, 309-313). Mutations of the phospho-acceptor serine residues within the MAPKK activation loop alters IKK2 kinase activity; the serine to alanine substitutions result in decreased kinase activity whereas the serine to glutamic acid substitutions result in a constitutively active kinase. Similar alanine mutations in IKK1 do not result in a decreased stimulation of total IKK activity in response to TNFα or IL1β (Dehase, M., Hayakawa, M., Chen, Y., and Karin, M. (1999) Science 284, 309-313). IKK2 being the dominant kinase activity within the IKK complex is further supported by the analysis of fibroblasts from mice deficient in IKK1 or IKK2. Fibroblasts lacking IKK1 retain full IKK activity in response to cytokines and could activate NF-κB. In contrast, fibroblasts lacking IKK2 do not exhibit IKK activity when stimulated with cytokines nor do they activate NF-κB. Furthermore, the phenotypes of each IKK knock out is unique with IKK1 deficiency resulting in skin and skeletal defects and IKK2 knock out being embryonic lethal due to hepatocyte apoptosis (Li, Q., Antwerp, D. V., Mercurio, F., Lee, K., and Verma, I. M. (1999) Science 284, 321-325; Takeda, K., Tekeuchi, O., Tsujimura, T., Itami, S., Adachi, O., Kawai, T., Sanjo, H., Yoshikawa, K., Terada, N, and Akira, S. (1999) Science 284, 313-316; Hu, Y., Baud, V., Delhase, M., Zhang, P., Deerinck, T., Ellisman, M., Johnson, R., and Karin, M. (1999) Science 284, 315-320; Li, Q., Lu, Q., Hwang, J. Y., Buscher, D., Lee, K., Izpisua-Belmonte, J. C., and Verma, I. M. (1999) Gene and Development 13, 1322-1328; Tanaka, M., Fuentes, M. E., Yamaguchi, K., Durnin, M. H., Dalrymple, S. A., Hardy, K. L., and Goeddel, D. V. (1999) Immunity 10, 421-429).

It is well-known that NF-κB plays a key role in the regulated expression of a large number of pro-inflammatory mediators including cytokines such as IL-6 and IL-8, cell adhesion molecules, such as ICAM and VCAM, and inducible nitric oxide synthase (iNOS). Such mediators are known to play a role in the recruitment of leukocytes at sites of inflammation and in the case of iNOS, may lead to organ destruction in some inflammatory and autoimmune diseases. The importance of NF-κB in inflammatory disorders is further strengthened by studies of airway inflammation including asthma in which NF-κB has been shown to be activated. This activation may underlie the increased cytokine production and leukocyte infiltration characteristic of these disorders. In addition, inhaled steroids are known to reduce airway hyperresponsiveness and suppress the inflammatory response in asthmatic airways. In light of the recent findings with regard to glucocorticoid inhibition of NF-κB, one may speculate that these effects are mediated through an inhibition of NF-κB. Further evidence for a role of NF-κB in inflammatory disorders comes from studies of rheumatoid synovium. Although NF-κB is normally present as an inactive cytoplasmic complex, recent immunohistochemical studies have indicated that NF-κB is present in the nuclei, and hence active, in the cells comprising rheumatoid synovium. Furthermore, NF-κB has been shown to be activated in human synovial cells in response to stimulation with TNF-α. Such a distribution may be the underlying mechanism for the increased cytokine and eicosanoid production characteristic of this tissue. See Roshak, A. K., et al., J. Biol. Chem., 271, 31496-31501 (1996).

The NF-κB/Rel and IκB proteins are also likely to play a key role in neoplastic transformation. Family members are associated with cell transformation in vitro and in vivo because of overexpression, gene amplification, gene rearrangements, or translocations (Gilmore T D, Trends Genet 7:318-322, 1991; Gillmore T D, Oncogene 18:6925-6937, 1999; Rayet B. et al., Oncogene 18: 6938-6947, 1991). In addition, rearrangement and/or amplification of the genes encoding these proteins are seen in 20-25% of certain human lymphoid tumors. In addition, a role for NFκB in the regulation of apoptosis, cell cycle progression, invasion, and metastasis has been reported (Bours V. et al., Biochemical Pharmacology 60:1085-1090, 2000) strengthening the role of this transcription factor in the control of cell proliferation. The inhibition of NF-κB has been shown to potentiate TNF- and cancer therapy through increased apoptosis (Wang C-Y et al., Science 274:784-787, 1996; Wang C-Y et al., Nat Med 5:412-417, 1999). It has also been shown that human T-cell leukemia virus type 1 (HTLV 1) infected cells (the etiological agent of an aggressive malignancy of activated CD4⁺ T lymphocytes), IKKα and IKK0 are expressed constitutively, which normally function in a transient manner (Chu Z-L et al., J of Biological Chemistry 273:15891-15894, 1998). The HTLV1 transforming and transactivating protein (Tax) has been shown to bind MEKK1 and increases the activity of IKKβ to enhance phosphorylation of serine residues in IκBα that lead to its degradation.

U.S. Pat. No. 4,999,436 discloses aryl and heterocyclic substituted thiophene derivatives as inhibitors of 5-lipoxygenase for treating inflammation.

U.S. Pat. No. 4,797,414 discloses thienobenzothiopyran derivatives as respiratory enhancing agents.

U.S. Pat. No. 5,468,750 discloses heterocycle-coupled substituted pyrrolo[3,2—C]pyridin-2-carbxylic acids as inhibitors of the biological effect of oxygenated free radicals.

### DETAILED DESCRIPTION OF THE INVENTION

The present invention concerns novel pharmaceutical composition comprising tricyclic compounds of the Formula I

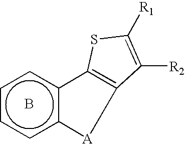

or isomers, tautomers, polymorphs, carriers, prodrugs, pharmaceutically acceptable salts thereof, wherein;

A is (CH₂)ₘ or (CH₂)ₘ—W—(CH₂)ₙ, wherein A is optionally substituted with one or more substituent independently selected from the group consisting of sulfamyl, halo, alkyl, alkoxy, hydroxyl and haloalkyl, CF₃, COCF₃, CN, NO₂, hydrido, OR³, OCOOR³, CO₂H, CO₂R³, CONH₂, CONHR³, CON(R³)₂, COR³, SR³, SOR³, SCOOR³, SO₂R³, NH₂, NHR³, NR³R³, NR³COR³, NR³CONHR, NR³SO₂R³, NR³SO₂NHR³, SO₂NHR³, and SO₂N(R³)₂;

B is a 6-membered aromatic hydrocarbon ring, optionally substituted with one or more substituent independently selected from the group consisting of OR³, SR⁴, SO₂N(R⁴)₂, NHR⁴, NHCOR⁴, NR⁴COR⁴, NHCO(OR⁴), NR⁴CO(OR⁴), NHCONHR³, NR³CONHR⁴, NR³SO₂R⁴, NHSO₂R⁴, NHSO₂N(R⁴)₂, NHCONR⁴R⁴, CO₂R⁴, CON(R⁴)₂, aryl, heteroaryl, heterocyclic, halo, alkylthio, alkylsulfinyl, alkylsulfonyl, cyano, carboxyl, alkoxycarbonyl, amido, N-monoalkylamido, N-monoarylamido, alkyl, alkenyl, alkynyl, N,N-dialkylamido, N-alkyl-N-arylamido, haloalkyl, hydrido, hydroxyl, alkoxy, hydroxyalkyl, haloalkoxy, sulfamyl, N-alkylsulfamyl, amino, alkylamino, and nitro;

W is S(O)ₚ, O, CH═N, N(O)═CH, CH₂═CH₂, and NR⁴;

m is 0 to 3, inclusive;

n is 0 to 3, inclusive;

p is 0 to 2, inclusive;

R¹ and R² are independently selected from the group consisting of: hydrido, cyano, nitro, hydroxyl, alkyl, hydroxyalkyl, haloalkyl, alkoxy, haloalkoxy, amidine, guanidine, CONHR⁵, NHR⁵, and NHCXNHR⁵, wherein X is O, S, or NR⁶;

R³ is selected from the group consisting of: hydrido, aryl, heteroaryl, lower alkyl, alkenyl, alkynyl, and heteroalkyl;

R⁴ is selected from the group consisting of: lower alkyl, aryl, heteroaryl, arylalkyl, heteroalkyl, haloalkyl, arylalkylamino, and heteroarylalkyl, wherein aryl, arylalkyl, heteroaryl, or heteroarylalkyl are optionally substituted with one or more radical selected from alkyl, alkoxy, halo, haloalkyl, cyano, haloalkoxy, acyl, carboxyl, hydroxy, hydroxyalkyloxy, phenoxy, benzyloxy, dialkylaminoalkyloxy, and heterocyclic;

R⁵ is selected from the group consisting of: hydrido, alkyl, aminoalkyl, hydroxyalkyl, haloalkyl, and acyl;

R⁶ is selected from the group consisting of: hydrido, alkyl, cyano, and nitro.

With the proviso, when W is CH═N and m is 0, the nitrogen atom in CH═N has to be directly attached to the ring B.

Definitions

The present invention includes the use of all hydrates, solvates, complexes and prodrugs of the compounds of this invention. Prodrugs are any covalently bonded compounds, which release the active parent drug according to Formula I in vivo. If a chiral center or another form of an isomeric center is present in a compound of the present invention all forms of such isomer or isomers, including enantiomers and diastereomers, are intended to be covered herein. Compounds containing a chiral center may be used as a racemic mixture, an enantiornerically enriched mixture, or the racemic mixture may be separated using well-known techniques and an individual enantiomer may be used alone. In cases in which compounds have unsaturated carbon-carbon double bonds, both the cis (Z) and trans (E) isomers are within the scope of this invention. In cases wherein compounds may exist in tautomeric forms, such as keto-enol tautomers, each tautomeric form is contemplated as being included within this invention whether existing in equilibrium or predominantly in one form.

The meaning of any substituent at any one occurrence in Formula I or any sub-formula thereof is independent of its meaning, or any other substituents meaning, at any other occurrence, unless specified otherwise.

The term “alkyl” is used, either alone or within other terms such as “haloalkyl” and “alkylsulfonyl”; it embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about five carbon atoms. Examples of such radicals include methyl, ethyl, n-propyl, isopropyl, n-butyl, isobutyl, sec-butyl, tert-butyl, pentyl, isoamyl, hexyl, octyl and the, like. The term “hydrido” denotes a single hydrogen atom (H). This hydrido radical may be attached, for example, to an oxygen atom to form a hydroxyl radical or two hydrido radicals may be attached to a carbon atom to form a methylene (—CH₂—) radical. The term “halo” means halogens such as fluorine, chlorine, and bromine or iodine atoms. The term “haloalkyl” embraces radicals wherein any one or more of the alkyl carbon atoms is substituted with halo as defined above. Specifically embraced are monohaloalkyl, dihaloalkyl, and polyhaloalkyl radicals. A monohaloalkyl radical, for one example, may have a bromo, chloro, or a fluoro atom within the radical. Dihalo radicals may have two or more of the same halo atoms or a combination of different halo radicals and polyhaloalkyl radicals may have more than two of the same halo atoms or a combination of different halo radicals. The term “hydroxyalkyl” embraces linear or branched alkyl radicals having one to about ten carbon atoms any one of which may be substituted with one or more hydroxylradicals. The terms “alkoxy” and “alkoxyalkyl” embrace linear or branched oxy-containing radicals each having alkyl portions of one to about ten carbon atoms, such as methoxy radical. The term “alkoxyalkyl” also embraces alkyl radicals having two or more alkoxy radicals attached to the alkyl radical, that is, to form monoalkoxyalkyl and dialkoxyalkyl radicals. The “alkoxy” or “alkoxyalkyl” radicals may be further substituted with one or more halo atoms, such as fluoro, chloro, or bromo, to provide “haloalkoxy” or “haloalkoxyalkyl” radicals. Examples of “alkoxy” radicals include methoxy, butoxy, and trifluoromethoxy. The term “aryl”, alone or in combination, means a carbocyclic aromatic system containing one, two, or three rings wherein such rings may be attached together in a pendent manner or may be fused. The term “aryl” embraces aromatic radicals such as phenyl, naphthyl, tetrahydronapthyl, indane, and biphenyl. The term “heterocyclic” embraces saturated, partially saturated, and unsaturated heteroatom-containing ring-shaped radicals, where the heteroatoms may be selected from nitrogen, sulfur and oxygen. Examples of saturated heterocyclic radicals include pyrrolidyl and morpholinyl. The term “heteroaryl” embraces unsaturated heterocyclic radicals. Examples of unsaturated heterocyclic radicals, also termed “heteroaryl” radicals include thienyl, pyrrolyl, furyl, pyridyl, pyrimidyl, pyrazinyl, pyrazolyl, oxazolyl, isoxazolyl, imidazolyl, thiazolyl, and tetrazolyl. The term also embraces radicals where heterocyclic radicals are fused with aryl radicals. Examples of such fused bicyclic radicals include benzofuran, benzothiophene, and the like. The term “sulfonyl”, whether used alone or linked to other terms such as alkylsulfonyl, denotes respectively divalent radicals —SO₂—. “Alkylsulfonyl”, embraces alkyl radicals attached to a sulfonyl radical, where alkyl is defined as above. The term “arylsulfonyl” embraces sulfonyl radicals substituted with an aryl radical. The terms “sulfamyl” or “sulfonamidyl”, whether alone or used with terms such as “N-alkylsulfamyl”, “N-arylsulfamyl”, “N,N-dialkylsulfamyl” and “N-alkyl-N-arylsulfamyl”, denotes a sulfonyl radical substituted with an amine radical, forming a sulfonamide (—SO₂NH₂). The terms “N-alkylsulfamyl” and “N,N-dialkylsulfamyl” denote sulfamyl radicals substituted, respectively, with one alkyl radical, a cycloalkyl ring, or two alkyl radicals. The terms “N-arylsulfamyl” and “N-alkyl-N-arylsulfamyl” denote sulfamyl radicals substituted, respectively, with one aryl radical, and one alkyl and one aryl radical. The terms “carboxy” or “carboxyl”, whether used alone or with other terms, such as “carboxyalkyl”, denotes —CO₂H. The term “carboxyalkyl” embraces radicals having a carboxyradical as defined above, attached to an alkyl radical. The term “carbonyl”, whether used alone or with other terms, such as “alkylcarbonyl”, denotes —C(═O)—. The term “alkylcarbonyl” embraces radicals having a carbonyl radical substituted with an alkyl radical. An example of an “alkylcarbonyl” radical is CH₃—C(═O)—. The term “alkylcarbonylalkyl” denotes an alkyl radical substituted with an “alkylcarbonyl” radical. The term “alkoxycarbonyl” means a radical containing an alkoxy radical, as defined above, attached via an oxygen atom to a carbonyl (C═O) radical. Examples of such “alkoxycarbonyl” radicals include (CH₃)₃CO—C(═O)— and —(O═)C—OCH₃. The term “alkoxycarbonylalkyl” embraces radicals having “alkoxycarbonyl”, as defined above substituted to an alkyl radical. Examples of such “alkoxycarbonylalkyl” radicals include (CH₃)₃COC(═O)—(CH₂)₂— and —(CH₂)₂ (O═)COCH₃. The term “amido” when used by itself or with other terms such as “amidoalkyl”, “N-monoalkylamido”, “N-monoarylamido”, “N,N-dialkylamido”, “N-alkyl-N-arylamido”, “N-alkyl-N-hydroxyamido” and “N-alkyl-Nhydroxyamidoalkyl”, embraces a carbonyl radical substituted with an amino radical. The terms “N-alkylamido” and “N,N-dialkylamido” denote amido groups which have been substituted with one alkyl radical and with two alkyl radicals, respectively. The terms “N-monoarylamido” and “N-alkyl-N-arylamido” denote amido radicals substituted, respectively, with one aryl radical, and one alkyl and one aryl radical. The term “N-alkyl-N-hydroxyamido” embraces amido radicals substituted with a hydroxyl radical and with an alkyl radical. The term “N-alkyl-Nhydroxyamidoalkyl” embraces alkyl radicals substituted with an N-alkyl-N-hydroxyamido radical. The term “amidoalkyl” embraces alkyl radicals substituted with amido radicals. The term “aminoalkyl” embraces alkyl radicals substituted with amino radicals. The term “alkylaminoalkyl” embraces aminoalkyl radicals having the nitrogen atom substituted with an alkyl radical. The term “amidino” denotes an —C(═NH)—NH₂ radical. The term “cyanoamidino” denotes an —C(═N—CN)—NH₂ radical. The term “heterocycloalkyl” embraces heterocyclic-substituted alkyl radicals such as pyridylmethyl and thienylmethyl. The term “aralkyl” embraces aryl-substituted alkyl radicals such as benzyl, diphenylmethyl, triphenylmethyl, phenethyl, and diphenethyl. The terms benzyl and phenylmethyl are interchangeable. The term “cycloalkyl” embraces radicals having three to ten carbon atoms, such as cyclopropyl cyclobutyl, cyclopentyl, cyclohexyl, and cycloheptyl. The term “cycloalkenyl” embraces unsaturated radicals having three to ten carbon atoms, such as cylopropenyl, cyclobutenyl, cyclopentenyl, cyclohexenyl, and cycloheptenyl. The term “alkylthio” embraces radicals containing a linear or branched alkyl radical, of one to ten carbon atoms, attached to a divalent sulfur atom. An example of “alkylthio” is methylthio, (CH₃S—). The term “alkylsulfinyl” embraces radicals containing a linear or branched alkyl radical, of one to ten carbon atoms, attached to a divalent —S(O)— atom. The terms “N-alkylamino” and “N,N-dialkylamino” denote amino groups which have been substituted with one alkyl radical and with two alkyl radicals, respectively. The term “acyl”, whether used alone, or within a term such as “acylamino”, denotes a radical provided by the residue after removal of hydroxyl from an organic acid. The term “acylamino” embraces an amino radical substituted with an acyl group. An example of an “acylamino” radical is acetylamino (CH₃C(═O)—NH—).

Compounds of Formula I or would be useful for, but not limited to, the treatment of inflammation in a subject, and for treatment of other inflammation-associated disorders, such as, as an analgesic in the treatment of pain and headaches, or as an antipyretic for the treatment of fever. For example, compounds of Formula I would be useful to treat arthritis, including but not limited to rheumatoid arthritis, spondylo arthopathies, gouty arthritis, osteoarthritis, systemic lupus erythematosus, and juvenile arthritis. Such compounds of Formula I would be useful in the treatment of asthma, bronchitis, menstrual cramps, tendinitis, bursitis, and skin related conditions such as psoriasis, eczema, burns, and dermatitis. Compounds of Formula I also would be useful to treat gastrointestinal conditions such as inflammatory bowel disease, Crohn's disease, gastritis, irritable bowel syndrome, and ulcerative colitis and for the prevention of colorectal cancer. Compounds of Formula I would be useful in treating inflammation in such diseases as vascular diseases such as vascularitus, migraine headaches, periarteritis nodosa, thyroiditis, aplastic anemia, Hodgkin's disease, sclerodoma, rheumatic fever, type I diabetes, myasthenia gravis, sarcoidosis, nephrotic syndrome, Behcet's syndrome, polymyositis, gingivitis, hypersensitivity, conjunctivitis, swelling occurring after injury, myocardial ischemia, and the like. The compounds of the present invention may also be used for pain. The compounds are useful as antiinflammatory agents, such as for the treatment of arthritis, with the additional benefit of having significantly less harmful side effects. The compounds of Formula I are useful as agents for treating cancer or anticancer agents. The compounds of Formula I may be proapoptotic, antiapoptotic, anticell cycle progressive, antiinvasive, antiproliferative, antiangiogenic, and antimetastatic. The cancer may be colon, ovarian, breast, prostate, gastric, B-cell lymphoma, and multiple myeloma. The compounds of Formula I may be used as an anitviral agent. The compounds of this invention may act as inhibitors of protein kinases. The compounds of this invention may act as inhibitors of IKK1 and/or IKK2, IKKα/IKKβ heterodimer, TBK or IKKi. The present invention preferably includes compounds, which selectively inhibit IKK2 over IKK1. Preferably, the compounds have an IKK2 IC50 of less than 1 μM, and have a selectivity ratio of IKK2 inhibition over IKK1 inhibition of at least 50, and more preferably of at least 100. Even more preferably, the compounds have an IKK1 IC50 of greater than 10 μM, and more preferably of greater than 100 μM. The compounds of formula may also be used to treat angiogenesis associated cardiovascular, ophthalmology and osteoporosis disorders. The compounds of the present invention may also be used for treatment of knee injury such as sport injuries.

While it is possible for an active ingredient to be administered alone as the raw chemical, it is preferable to present it as a pharmaceutical formulation. The present invention comprises a pharmaceutical composition comprising a therapeutically effective amount of a compound of the present invention in association with at least one pharmaceutically acceptable carrier, adjuvant, or diluent. The present invention also comprises a method of treating inflammation or inflammation associated disorders in a subject, the method comprising administering to the subject having such inflammation or disorders a therapeutically effective amount of a compound of the present invention. Also included in the family of compounds of the present invention are the pharmaceutically acceptable salts thereof. The term “pharmaceutically acceptable salts” embraces salts commonly used to form alkali metal salts and to form addition salts of free acids or free bases. The nature of the salt is not critical, provided that it is pharmaceutically acceptable. Suitable pharmaceutically acceptable acid addition salts of compounds of the present invention may be prepared from an inorganic acid or from an organic acid. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamric, benzoic, anthranilic, mesylic, salicyclic, salicyclic, phydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, cyclohexylaminosulfonic, algenic, β-hydroxybutyric, salicyclic, galactaric and galacturonic acid. Suitable pharmaceutically acceptable base addition salts of compounds of the present invention include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine (N-methyl-glucamine) and procaine. All of these salts may be prepared by conventional means from the corresponding compound of the present invention by reacting, for example, the appropriate acid or base with the compound of the present invention.

Also embraced within this invention are pharmaceutical compositions comprising one or more compounds of the present invention in association with one or more non-toxic, pharmaceutically acceptable carriers and/or diluents and/or adjuvants and/or excipient (collectively referred to herein as “carrier” materials) and, if desired, other active ingredients. Accordingly, the compounds of the present invention may be used in the manufacture of a medicament. Pharmaceutical compositions of the compounds of the present invention prepared as herein before described may be formulated as solutions or lyophilized powders for parenteral administration. Powders may be reconstituted by addition of a suitable diluent or other pharmaceutically acceptable carrier prior to use. The liquid formulation may be a buffered, isotonic aqueous solution. The compounds of the present invention may be administered by any suitable route, preferably in the form of a pharmaceutical composition adapted to such a route, and in a dose effective for the treatment intended. The compounds and composition may, for example, be administered intravascularly, intraperitoneally, intravenously, subcutaneously, intramuscularly, intramedullary, orally, or topically. For oral administration, the pharmaceutical composition may be in the form of, for example, a tablet, capsule, suspension, or liquid. The active ingredient may also be administered by injection as a composition wherein, for example, normal isotonic saline solution, standard 5% dextrose in water or buffered sodium or ammonium acetate solution may be used as a suitable carrier. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride, or sodium citrate. The pharmaceutical composition is preferably made in the form of a dosage unit containing a particular amount of the active ingredient. Examples of such dosage units are tablets or capsules. The amount of therapeutically active compound that is administered and the dosage regimen for treating a disease condition with the compounds and/or compositions of this invention depends on a variety of factors, including the age, weight, sex and medical condition of the subject, the severity of the disease, the route and frequency of administration, and the particular compound employed, and thus may vary widely. The pharmaceutical compositions may contain active ingredient in the range of about 0.1 to 2000 mg, preferably in the range of about 0.5 to 500 mg and most preferably between about 1 and 100 mg. A daily dose of about 0.01 to 100 mg/kg bodyweight, preferably between about 0.1 and about 50 mg/kg body weight and most preferably between about 1 to 20 mg/kg bodyweight, may be appropriate. The daily dose can be administered in one to four doses per day. For therapeutic purposes, the compounds of this invention are ordinarily combined with one or more adjuvants appropriate to the indicated route of administration. If administered orally, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled release formulation as may be provided in a dispersion of active compound in a sustained release material such as glyceryl monostearate, glyceryl distearate, hydroxypropylmethyl cellulose alone or with a wax. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion, or an aqueous or non-aqueous suspension. Such a liquid formulation may be administered orally or filled into a soft gelatin capsule. For rectal administration, the compounds of the present invention may also be combined with excipients such as cocoa butter, glycerin, gelatin, or polyethylene glycols and molded into a suppository. The methods of the present invention include topical administration of the compounds of the present invention. By topical administration is meant non-systemic administration, including the application of a compound of the invention externally to the epidermis, to the buccal cavity and instillation of such a compound into the ear, eye, and nose, wherein the compound does not significantly enter the blood stream. By systemic administration is meant oral, intravenous, intraperitoneal, and intramuscular administration. The amount of a compound of the present invention (hereinafter referred to as the active ingredient) required for therapeutic or prophylactic effect upon topical administration will, of course, vary with the compound chosen, the nature and severity of the condition being treated and the animal undergoing treatment, and is ultimately at the discretion of the physician. The topical formulations of the present invention, both for veterinary and for human medical use, comprise an active ingredient together with one or more acceptable carriers therefore, and optionally any other therapeutic ingredients. The carrier must be “acceptable” in the sense of being compatible with the other ingredients of the formulation and not deleterious to the recipient thereof. Formulations suitable for topical administration include liquid or semi-liquid preparations suitable for penetration through the skin to the site of where treatment is required such as: liniments, lotions, creams, ointments or pastes, and drops suitable for administration to the eye, ear or nose. The active ingredient may comprise, for topical administration, from 0.01 to 5.0 wt % of the formulation. Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified by filtration, transferred to a suitable container, which is then sealed and sterilized by autoclaving, or maintaining at 90-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.00217c), benzalkonium chloride (0.0 1%) and chlorhexidine acetate (0.0 1%). Suitable solvents for the preparation of an oily solution include glycerol, diluted alcohol, and propylene glycol.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil. Creams, ointments, or pastes according to the present invention are semi-solid formulations of the active ingredient for external application. They may be made by mixing the active ingredient in finely divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogols. The formulation may incorporate any suitable surface-active agent such as an anionic, cationic, or non-ionic surface-active agent such as sorbitan esters or polyoxyethylene derivatives thereof. Suspending agents such as natural gums, cellulose derivatives or inorganic materials such as silicaceous silicas, and other ingredients such as lanolin may also be included. Other adjuvants and modes of administration are well and widely known in the pharmaceutical art. Although this invention has been described with respect to specific embodiments, the details of these embodiments are not to be construed as limitations.

General Synthetic Procedures

The starting materials used herein are commercially available or are prepared by routine methods well known to those of ordinary skill in the art and can be found in standard reference books, such as the COMPENDIUM OF ORGANIC SYNTHETIC METHODS, Vol. I-VI (published by Wiley-Interscience).

The compounds of the invention can be synthesized according to the following procedures of Scheme I, wherein the R¹-R⁴ substituents are as defined for Formula I, above, except where further noted.

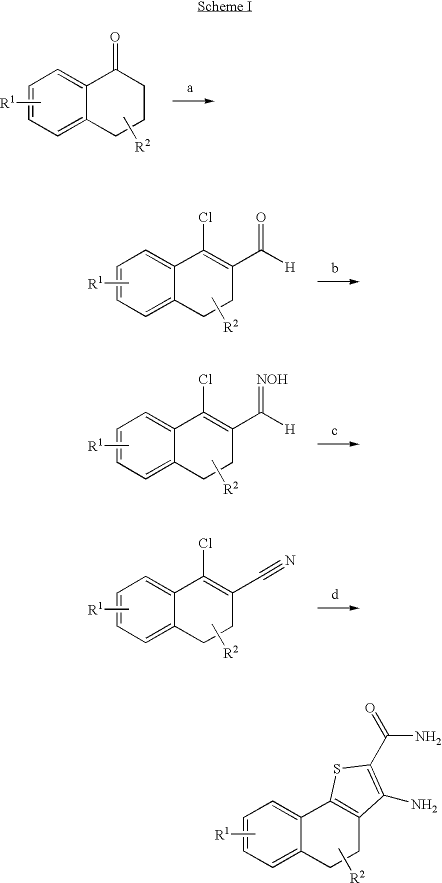

a) POCl₃, DMF 5° C.-RT, 16 h b) NH₂OH×HCl, NaOAc, EtOH (90%) reflux 2 h

c) Acetic anhydride, pyridine, reflux d) 2-Mercaptoacetamide, NaOMe, reflux

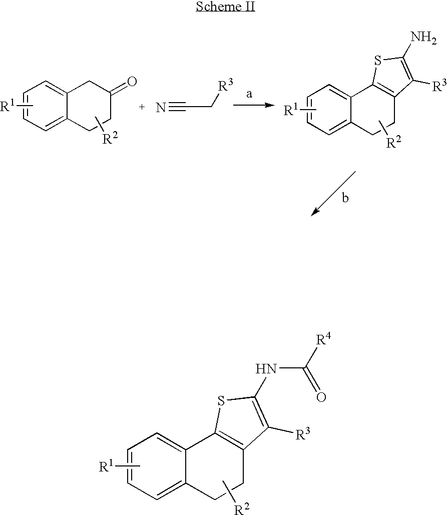

R³═CONH₂ or CN, R⁴═CH₃ or NH₂

a) sulfur, amine base, EtOH reflux, b) sodium cyanate in AcOH

The complete content of all publications, patents, and patent applications cited in this disclosure are herein incorporated by reference as if each individual publication, patent, or patent application were specifically and individually indicated to incorporated by reference. Although the foregoing invention has been described in some detail by way of illustration and example for the purposes of clarity of understanding, it will be readily apparent to one skilled in the art in light of the teachings of this invention that changes and modifications can be made without departing from the spirit and scope of the present invention. The following examples are provided for exemplification purposes only and are not intended to limit the scope of the invention, which has been described in broad terms above.

### EXAMPLES

### Examples 1

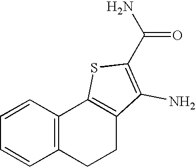

3-amino-4,5-dihydronaphtho[1,2-b]thiophene-2-carboxamide 1-chloro-3,4-dihydronaphthalene-2-carbaldehyde 1a: In a 3 neck flask fitted with a N₂ inlet tube, thermometer, and addition funnel, was placed DMF (8.4 g) and CH₂Cl₂ (40 mL). After cooling in an ice bath to 0-5° C., POCl₃ (14 g) was added dropwise. After the addition was complete the reaction was stirred at RT for 2 h. The mixture was then cooled to 0-5° C. and a solution of α-tetralone (8.76 g, 60 mmol) in CH₂Cl₂ (50 mL) was added. After the addition was complete the reaction was allowed to stir at RT for 16 h. The reaction solution was poured onto ice and NaHCO₃ (satd, 200 mL), added an additional CH₂Cl₂ and stirred until gas evolution ceased. The layers were separated, and the aqueous layer was extracted with CH₂Cl₂ (250 mL). The CH₂Cl₂ extracts were combined, washed with water and dried (Na₂SO₄), and concentrated in vacuo to yield the title compound as a red liquid (11.7 g., 96% yield).

1-chloro-3,4-dihydronaphthalene-2-carbaldehyde oxime 1b: A mixture of 1 (11.0 g), hydroxylamine hydrochloride (5.2 g) and sodium acetate (11.75 g) in ethanol (90 mL, 90%) was refluxed for 2 h. The reaction was cooled to RT, then poured onto ice water (90 mL). The resulting precipitate was collected by filtration to yield the title compound as a tan solid (12 g, 100% yield).

1-chloro-3,4-dihydronaphthalene-2-carbonitrile 1c: A mixture of 2 (2.08 g) and pyridine (0.4 mL) in acetic anhydride was refluxed for 10 h. The reaction solution was cooled and poured onto ice water. The resulting precipitate was collected by filtration to yield the title compound as a tan solid (1.28 g, 68% yield)

3-amino-4,5-dihydronaphtho[1,2-b]thiophene-2-carboxamide 1: 2-Mercaptoacetamide (480 mg, 5.27 mmol) was added to a freshly prepared solution of sodium methoxide (5.27 mmol) in methanol (20 mL). The resulting solution was stirred at RT for 15 minutes, followed by the addition of 3 (1.0 g, 5.27 mmol). The resulting solution was refluxed overnight. The solvent was removed in vacuo and the residue suspended in ethylacetate (50 mL) and HCl (1 N, 50 mL). The resulting solid was collected by filtration and recrystallized from methanol to yield the title compound as an off white solid (358 mg, 32%). The title compound 3-amino-4,5-dihydronaphtho[1,2-b]thiophene-2-carboxamide was evaluated in the IKK-2 Resin assay and the IC₅₀ was ≧10≦20 μM.

### Example 2

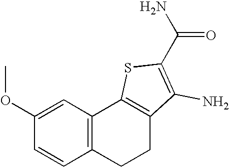

3-amino-8-methoxy-4,5-dihydronaphtho[1,2-b]thiophene-2-carboxamide

1-chloro-7-methoxy-3,4-dihydronaphthalene-2-carbaldehyde 2a: This material was prepared by the method of example 1a from 7-methoxy-α-tetralone (5 g) to yield the title compound as a red liquid. This material was used directly in the next step.

1-chloro-7-methoxy-3,4-dihydronaphthalene-2-carbaldehyde oxime Lb: A mixture of 2a (6.2 g), hydroxylamine hydrochloride (2.78 g) and sodium acetate (6.15 g) in ethanol (50 mL, 90%) was refluxed for 2.5 h. The reaction was cooled to RT, then poured onto ice water (50 mL). The resulting precipitate was collected by filtration to yield the title compound as a yellow solid (5.8 g, 86% yield from 7-methoxy-α-tetralone).

1-chloro-7-methoxy-3,4-dihydronaphthalene-2-carbonitrile 2c: A mixture of 2b (5.8 g) and pyridine (0.5 mL) in acetic anhydride (50 mL) was refluxed for 8 h. The reaction solution was cooled and poured onto ice water (250 mL). The resulting precipitate was collected by filtration to yield the title compound as a brown solid (5.7 g, 68% yield).

3-amino-8-methoxy-4,5-dihydronaphtho[1,2-b]thiophene-2-carboxamide 2: 2-Mercaptoacetamide (2.09 g) was added to a freshly prepared solution of sodium methoxide (23 mmol) in methanol (50 mL). The resulting solution was stirred at RT for 15 minutes, followed by the addition of 2c (5.0 g). The resulting solution was refluxed overnight. The solvent was removed in vacuo and the residue suspended in ethylacetate (50 mL) and HCl (1 N, 50 mL). The resulting solid (6.0 g) was collected by filtration and recrystallized from methanol to yield the title compound as an off white solid (1.1 g, 17%). The title compound 3-amino-8-methoxy-4,5-dihydronaphtho[1,2-b]thiophene-2-carboxamide was evaluated in the IKK-2 Resin assay and the IC₅₀ was ≧1≦10 μM.

### Example 3

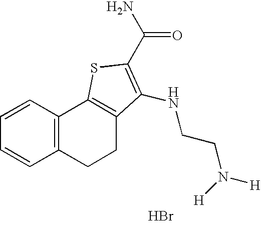

### 3-[(2-aminoethyl)amino]-4,5-dihydronaphtho[1,2-b]thiophene-2-carboxamide hydrobromide

A solution of 1 (732 mg) and 2-bromoethylamine hydrobromide (1.89 g) in DMF was heated at 80° C. for 16 h. The solvent was removed and the residue was purified by reverse phase HPLC to yield the title compound. The title compound 3-[(2-aminoethyl)amino]-4,5-dihydronaphtho[1,2-b]thiophene-2-carboxamide hydrobromide was evaluated in the IKK-2 Resin assay and the IC₅₀ was ≧1≦10 μM.

### Example 4

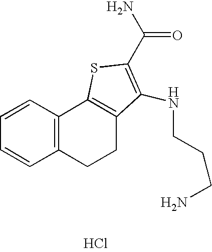

### 3-[(3-aminopropyl)amino]-4,5-dihydronaphtho[1,2-b]thiophene-2-carboxamide hydrochloride

A solution of 1(732 mg) and 2-bromopropylamine hydrobromide (2.0 g) in DMF was heated at 80° C. for 16 h. The solvent was removed and the residue was purified by reverse phase HPLC. The residue was lyophilized from 1N HCl to yield the title compound. The title compound of 3-[(3-aminopropyl)amino]4,5-dihydronaphtho[1,2-b]thiophene-2-carboxamide hydrochloride was evaluated in the IKK-2 Resin assay and the IC₅₀ was ≧10≦20 μM.

### Example 5

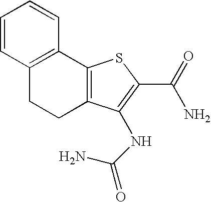

### 3-[(aminocarbonyl)amino]4,5-dihydronaphtho[1,2-b]thiophene-2-carboxamide

To a stirred solution of 1 (512 mg) in acetic acid (35 mL) and water (7 mL) was added sodium cyanate. The resulting solution was stirred for 16 h. The resulting solid was removed by filtration. The solid was suspended in ethanol (95%, 50 mL) and filtered and dried to yield the title compound. The title compound 3-[(aminocarbonyl)amino]-4,5-dihydronaphtho[1,2-b]thiophene-2-carboxamide was evaluated in the IKK-2 Resin assay and the IC₅₀ was ≧10≦20 μM.

### Example 6

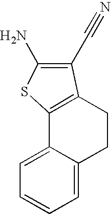

### 2-amino-4,5-dihydronaphtho[1,2-b]thiophene-3-carbonitrile

To a suspension of β-tetralone (22.6 g, 0.154 mol), malonitrile (20 mL, 0.16 mol) and sulfur (7.2 g, 0.22 mol) in ethanol (160 mL) was added morpholine (5 mL) dropwise. The reaction mixture was refluxed for 15 min and then another 150 mL of ethanol was added. The hot suspension as filtered and the solid was treated with a mixture of acetone and ether. The residual sulfur was removed by filtration and the filtrate was concentrated to give 13.3 g of desired product as a tan solid (33% yield). The title compound 2-amino-4,5-dihydronaphtho[1,2-b]thiophene-3-carbonitrile was evaluated in the IKK-2 Resin assay and the IC₅₀ was ≧10≦20 μM.

### Example 7

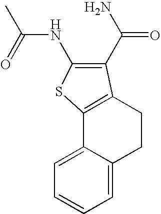

### 2-(acetylamino)-4,5-dihydronaphtho[1,2-b]thiophene-3-carboxamide

To a suspension of β-tetralone (11.3 g, 0.077 mol), cyanoacetamide (5.34 g, 0.08 mol) and sulfur (3.6 g, 0.11 mol) in ethanol (40 mL) was added diethylamine (2.5 mL) dropwise at room temperature. The reaction mixture was refluxed for 3 h and then cooled. The precipitate was collected by filtration, washed with ether and air-dried to give 1.47 g of 2-amino-4,5-dihydronaphtho[1,2-b]thiophene-3-carboxamide as a light yellow solid (8% yield). To a solution of this compound (0.13 g, 0.00053 mol) in AcOH (5 mL) was added a solution of sodium cyanate (0.07 g, 0.0011 mol) in water (2 mL). The mixture was stirred at room temperature overnight. This crude solid was isolated by filtration and purified by preparative HPLC to give 0.01 g of the desired product as a white solid. The title compound 2(acetylamino)-4,5-dihydronaphtho[1,2-b]thiophene-3-carboxamide was evaluated in the IKK-2 Resin assay and the IC₅₀ was ≧10≦20 μM.

### Example 8

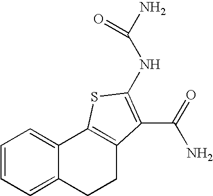

### 2-[(aminocarbonyl)amino]-4,5-dihydronaphtho[1,2-b]thiophene-3-carboxamide

To a solution of 2-amino-4,5-dihydronaphtho[1,2-b]thiophene-3-carboxamide (0.5 g, 0.002 mol) in AcOH (10 mL) was added a solution of sodium cyanate (0.135 g, 0.004 mol) in water (4 mL). The mixture was stirred at room temperature overnight. Another 4 eq of sodium cyanate was added and the solution was stirred for another 18 h. The precipitate was collected by filtration and purified by preparative HPLC to give 0.19 g of the desired product as a tan solid. The title compound 2-[(aminocarbonyl)amino]-4,5-dihydronaphtho[1,2-b]thiophene-3-carboxamide was evaluated in the IKK-2 Resin assay and the IC₅₀ was ≦1 μM.

The following compounds of the invention can be synthesized according to the Scheme III and to similar procedure as described in the preparation of example 8 using corresponding starting materials.

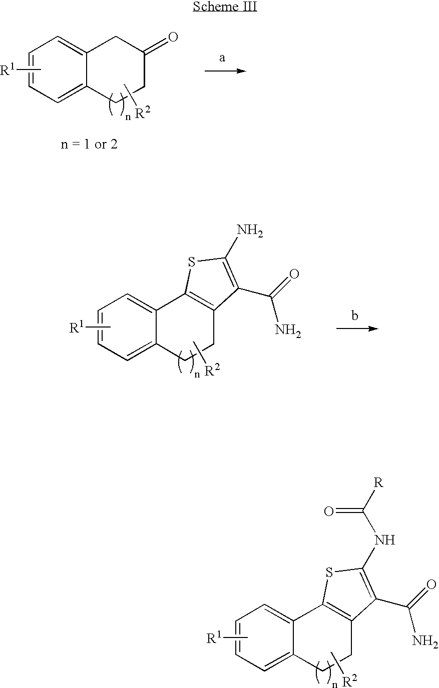

a) cyanoacetamide, sulfur, diethylamine b) sodium cyanate, AcOH.

| Example | | |
|---|---|---|
| number | Structure | Name |
| 9. | 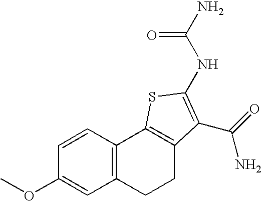 | 2-[(aminocarbonyl)- amino]-7-methoxy-4,5- dihydronaphtho-[1,2- b]thiophene-3- carboxamide |
| 10. | 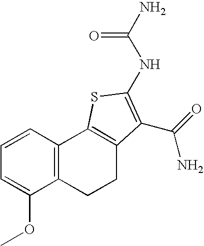 | 2-[(aminocarbonyl)- amino]-6-methoxy-4,5- dihydronaphtho-[1,2-b]- thiophene-3- carboxamide |
| 11. | 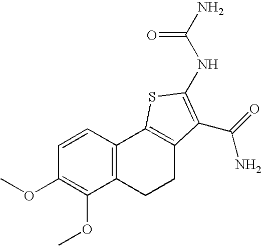 | 2-[(aminocarbonyl)- amino]-6,7-dimethoxy- 4,5-dihydronaphtho[1,2- b]thiophene-3- carboxamide |
| 12. | 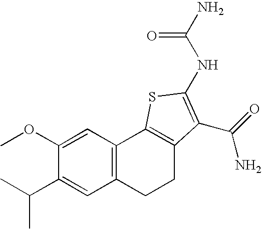 | 2-[(aminocarbonyl)- amino]-7-isopropyl-8- methoxy-4,5-dihydro- naphtho[1,2-b]- thiophene-3- carboxamide |
| 13. | 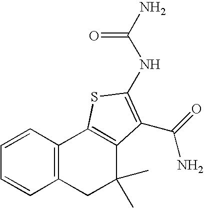 | 2-[(aminocarbonyl)- amino]-4,4-dimethyl- 4,5-dihydronaphtho- [1,2-b]thiophene-3- carboxamide |
| 14. | 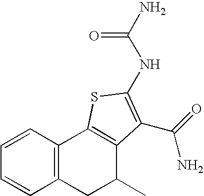 | 2-[(aminocarbonyl)- amino]-4-methyl-4,5- dihydronaphtho[1,2- b]thiophene-3- carboxamide |
| 15. | 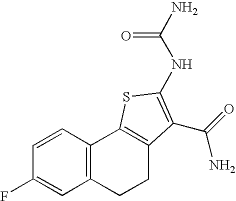 | 2-[(aminocarbonyl)- amino]-7-fluoro-4,5- dihydronaphtho[1,2- b]thiophene-3- carboxamide |
| 16. | 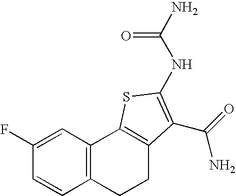 | 2-[(aminocarbonyl)amino]- 8-fluoro-4,5-dihydro- naphtho[1,2-b]- thiophene-3- carboxamide |
| 17. | 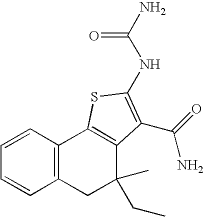 | 2-[(aminocarbonyl)- amino]-4-ethyl-4- methyl-4,5-dihydro- naphtho[1,2-b]- thiophene-3- carboxamide |
| 18. | 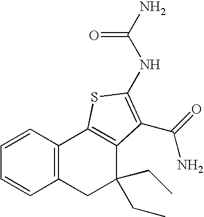 | 2-[(aminocarbonyl)- amino]-4,4-diethyl-4,5- dihydronaphtho[1,2-b]- thiophene-3- carboxamide |
| 19. | 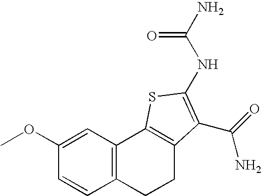 | 2-[(aminocarbonyl)- amino]-8-methoxy-4,5- dihydronaphtho[1,2-b]- thiophene-3- carboxamide |
| 20. | 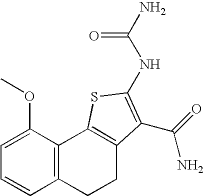 | 2-[(aminocarbonyl)- amino]-9-methoxy-4,5- dihydronaphtho[1,2-b]- thiophene-3- carboxamide |
| 21. | 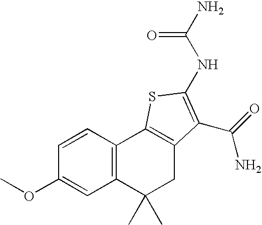 | 2-[(aminocarbonyl)- amino]-7-methoxy-5,5- dimethyl-4,5-dihydro- naphtho[1,2-b]- thiophene-3- carboxamide |
| 22. | 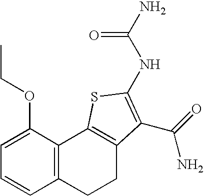 | 2-[(aminocarbonyl)- amino]-9-ethoxy-4,5- dihydronaphtho[1,2-b]- thiophene-3- carboxamide |
| 23. | 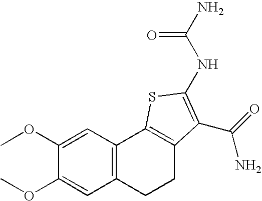 | 2-[(aminocarbonyl)- amino]-7,8-dimethoxy- 4,5-dihydronaphtho- [1,2-b]thiophene-3- carboxamide |
| 24. | 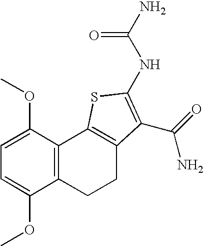 | 2-[(aminocarbonyl)- amino]-6,9-dimethoxy- 4,5-dihydronaphtho- [1,2-b]thiophene-3- carboxamide |
| 25. | 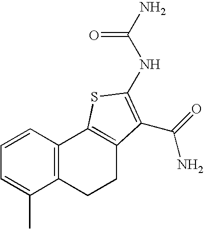 | 2-[(aminocarbonyl)- amino]-6-methyl-4,5- dihydronaphtho[1,2-b]- thiophene-3- carboxamide |
| 26. | 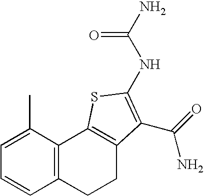 | 2-[(aminocarbonyl)- amino]-9-methyl-4,5- dihydronaphtho[1,2-b]- thiophene-3- carboxamide |
| 27. | 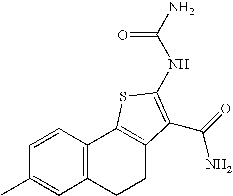 | 2-[(aminocarbonyl)- amino]-7-methyl-4,5- dihydronaphtho[1,2-b]- thiophene-3- carboxamide |
| 28. | 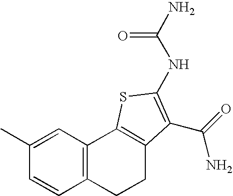 | 2-[(aminocarbonyl)- amino]-8-methyl-4,5- dihydronaphtho[1,2-b]- thiophene-3- carboxamide |
| 29. | 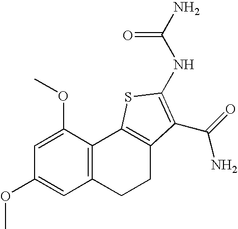 | 2-[(aminocarbonyl)- amino]-7,9-dimethoxy- 4,5-dihydronaphtho- [1,2-b]thiophene-3- carboxamide |
| 30. | 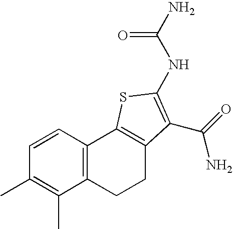 | 2-[(aminocarbonyl)- amino]-6,7-dimethyl- 4,5-dihydronaphtho- [1,2-b]thiophene-3- carboxamide |
| 31. | 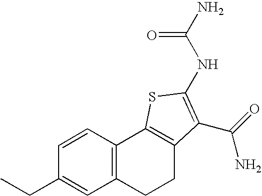 | 2-[(aminocarbonyl)- amino]-7-ethyl-4,5- dihydronaphtho[1,2-b]- thiophene-3- carboxamide |
| 32. | 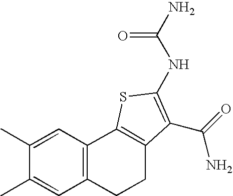 | 2-[(aminocarbonyl)- amino]-7,8-dimethyl- 4,5-dihydronaphtho- [1,2-b]thiophene-3- carboxamide |
| 33. | 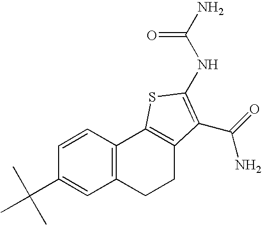 | 2-[(aminocarbonyl)- amino]-7-tert-butyl- 4,5-dihydronaphtho- [1,2-b]thiophene-3- carboxamide |
| 34. | 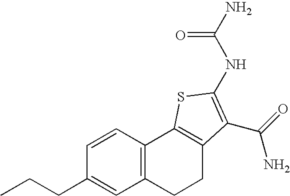 | 2-[(aminocarbonyl)- amino]-7-propyl-4,5- dihydronaphtho[1,2-b]- thiophene-3- carboxamide |
| 35. | 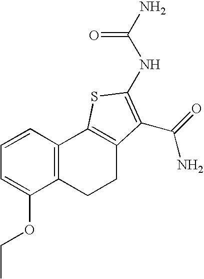 | 2-[(aminocarbonyl)- amino]-6-ethoxy-4,5- dihydronaphtho[1,2- b]thiophene-3- carboxamide |
| 36. | 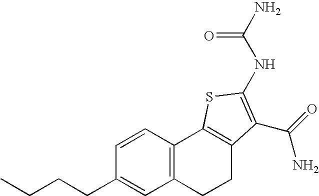 | 2-[(aminocarbonyl)- amino]-7-butyl-4,5- dihydronaphtho[1,2-b]- thiophene-3- carboxamide |
| 37. | 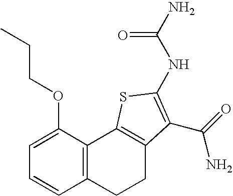 | 2-[(aminocarbonyl)- amino]-9-propoxy-4,5- dihydronaphtho[1,2-b]- thiophene-3- carboxamide |
| 38. | 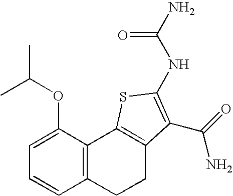 | 2-[(aminocarbonyl)- amino]-9-isopropoxy- 4,5-dihydronaphtho- [1,2-b]thiophene-3- carboxamide |
| 39. | 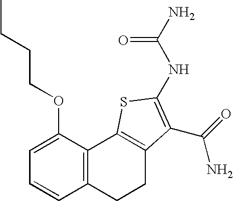 | 2-[(aminocarbonyl)- amino]-9-butoxy-4,5- dihydronaphtho[1,2- b]thiophene-3- carboxamide |
| 40. | 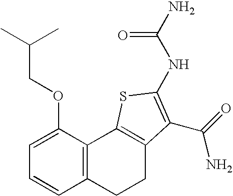 | 2-[(aminocarbonyl)- amino]-9-isobutoxy-4,5- dihydro-naphtho[1,2-b]- thiophene-3- carboxamide |
| 41. | 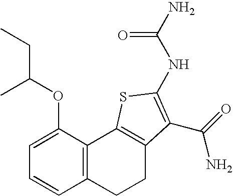 | 2-[(aminocarbonyl)- amino]-9-sec-butoxy- 4,5-dihydronaphtho- [1,2-b]thiophene-3- carboxamide |
| 42. | 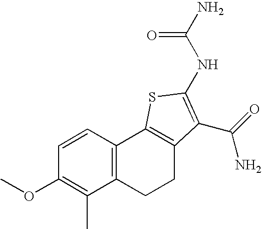 | 2-[(aminocarbonyl)- amino]-7-methoxy-6- methyl-4,5-dihydro- naphtho[1,2-b]- thiophene-3- carboxamide |
| 43. | 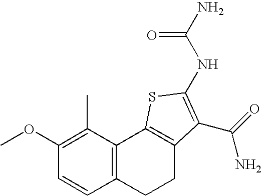 | 2-[(aminocarbonyl)- amino]-8-methoxy-9- methyl-4,5-dihydro- naphtho[1,2-b]- thiophene-3- carboxamide |
| 44. | 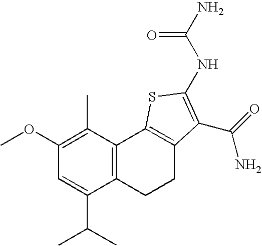 | 2-[(aminocarbonyl)- amino]-6-isopropyl-8- methoxy-9-methyl-4,5- dihydronaphthol[1,2-b]- thiophene-3- carboxamide |
| 45. | 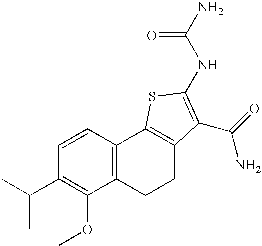 | 2-[(aminocarbonyl)- amino]-7-isopropyl-6- methoxy-4,5-dihydro- naphthol[1,2-b]- thiophene-3- carboxamide |
| 46. | 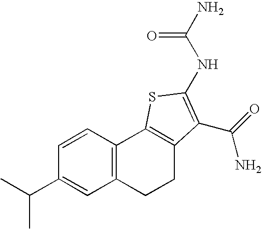 | 2-[(aminocarbonyl)- amino]-7-isopropyl- 4,5-dihydronaphtho- [1,2-b]-thiophene-3- carboxamide |
| 47. | 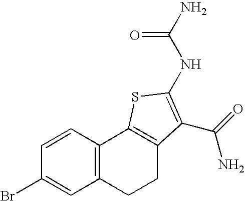 | 2-[(aminocarbonyl)- amino]-7-bromo-4,5- dihydronaphtho[1,2-b]- thiophene-3- carboxamide |
| 48. | 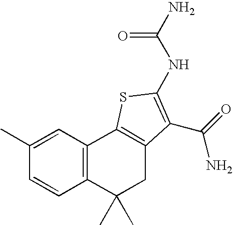 | 2-[(aminocarbonyl)- amino]-5,5,8-trimethyl- 4,5-dihydronaphtho- [1,2-b]thiophene-3- carboxamide |
| 49. | 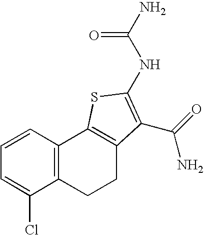 | 2-[(aminocarbonyl)- amino]-6-chloro-4,5- dihydronaphtho[1,2-b]- thiophene-3- carboxamide |
| 50. | 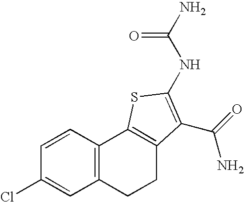 | 2-[(aminocarbonyl)- amino]-7-chloro-4,5- dihydronaphtho[1,2-b]- thiophene-3- carboxamide |
| 51. | 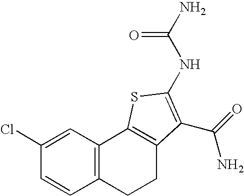 | 2-[(aminocarbonyl)- amino]-8-chloro-4,5- dihydronaphtho[1,2-b]- thiophene-3- carboxamide |
| 52. | 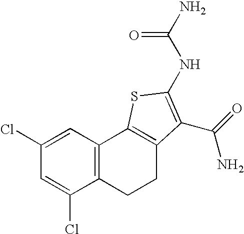 | 2-[(aminocarbonyl)- amino]-6,8-dichloro- 4,5-dihydronaphtho- [1,2-b]thiophene-3- carboxamide |
| 53. | 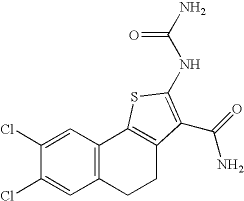 | 2-[(aminocarbonyl)- amino]-7,8-dichloro- 4,5-dihydronaphtho- [1,2-b]thiophene-3- carboxamide |
| 54. | 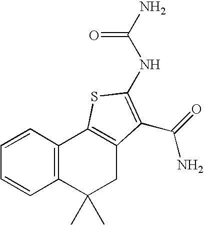 | 2-[(aminocarbonyl)- amino]-5,5-dimethyl- 4,5-dihydronaphtho- [1,2-b]thiophene-3- carboxamide |
| 55. | 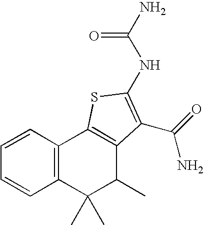 | 2-[(aminocarbonyl)- amino]-4,5,5-trimethyl- 4,5-dihydronaphtho- [1,2-b]thiophene-3- carboxamide |
| 56. | 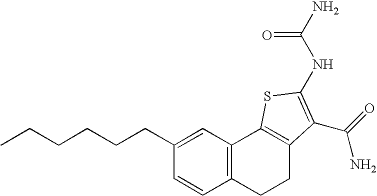 | 2-[(aminocarbonyl)- amino]-8-hexyl-4,5- dihydronaphtho[1,2-b]- thiophene-3- carboxamide |
| 57. | 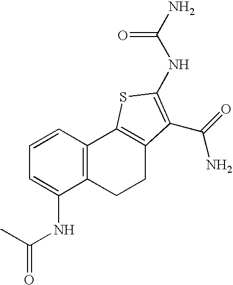 | 6-(acetylamino)-2- [(aminocarbonyl)amino]- 4,5-dihydronaphtho[1,2- b]thiophene-3- carboxamide |
| 58. | 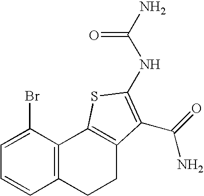 | 2-[(aminocarbonyl)- amino]-9-bromo-4,5- dihydronaphtho[1,2-b]- thiophene-3- carboxamide |
| 59. | 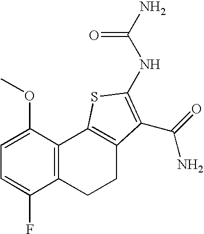 | 2-[(aminocarbonyl)- amino]-6-fluoro-9- methoxy-4,5-dihydro- naphtho[1,2-b]- thiophene-3- carboxamide |
| 60. | 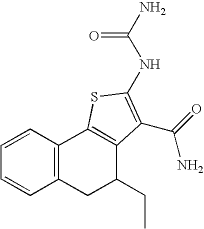 | 2-[(aminocarbonyl)- amino]-4-ethyl-4,5- dihydronaphtho[1,2-b]- thiophene-3- carboxamide |
| 61. | 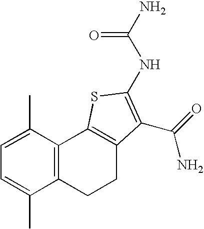 | 2-[(aminocarbonyl)- amino]-6,9-dimethyl- 4,5-dihydronaphtho[1,2- b]thiophene-3- carboxamide |
| 62. | 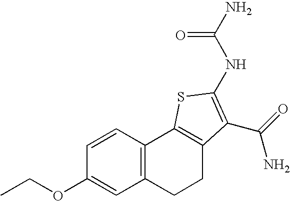 | 2-[(aminocarbonyl)- amino]-7-ethoxy-4,5- dihydronaphtho[1,2-b]- thiophene-3- carboxamide |
| 63. | 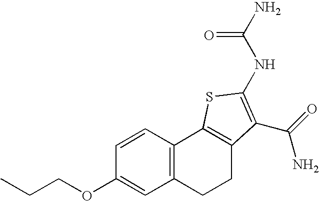 | 2-[(aminocarbonyl)- amino]-7-propoxy-4,5- dihydronaphtho[1,2-b]- thiophene-3- carboxamide |
| 64. | 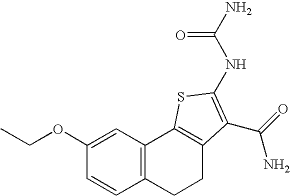 | 2-[(aminocarbonyl)- amino]-8-ethoxy-4,5- dihydronaphtho[1,2-b]- thiophene-3- carboxamide |
| 65. | 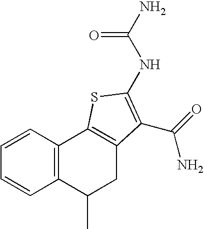 | 2-[(aminocarbonyl)- amino]-5-methyl-4,5- dihydronaphtho[1,2-b]- thiophene-3- carboxamide |
| 66. | 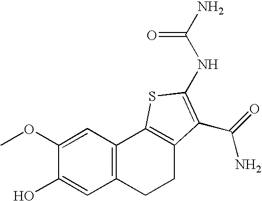 | 2-[(aminocarbonyl)- amino]-7-hydroxy-8- methoxy-4,5-dihydro- naphtho[1,2-b]- thiophene-3- carboxamide |
| 67. | 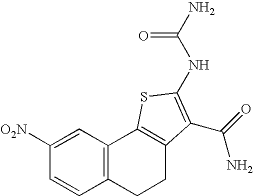 | 2-[(aminocarbonyl)- amino]-8-nitro-4,5- dihydronaphtho[1,2-b]- thiophene-3- carboxamide |
| 68. | 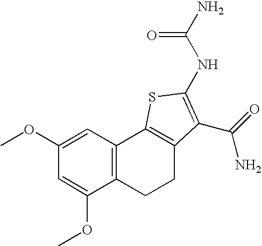 | 2-[(aminocarbonyl)- amino]-6,8-dimethoxy- 4,5-dihydronaphtho- [1,2-b]thiophene-3- carboxamide |
| 69. | 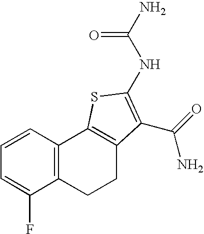 | 2-[(aminocarbonyl)- amino]-6-fluoro-4,5- dihydronaphtho[1,2-b]- thiophene-3- carboxamide |
| 70. | 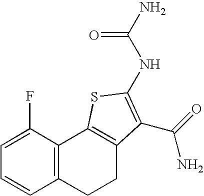 | 2-[(aminocarbonyl)- amino]-9-fluoro-4,5- dihydronaphtho[1,2-b]- thiophene-3- carboxamide |
| 71. | 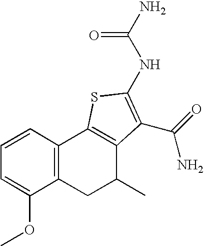 | 2-[(aminocarbonyl)- amino]-6-methoxy-4- methyl-4,5-dihydro- naphtho[1,2-b]- thiophene-3- carboxamide |
| 72. | 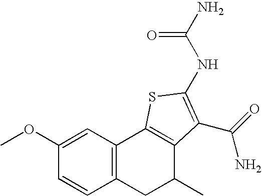 | 2-[(aminocarbonyl)- amino]-8-methoxy-4- methyl-4,5-dihydro- naphtho[1,2-b]- thiophene-3- carboxamide |
| 73. | 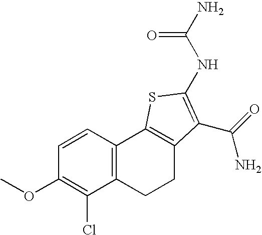 | 2-[(aminocarbonyl)- amino]-6-chloro-7- methoxy-4,5-dihydro- naphtho[1,2-b]- thiophene-3- carboxamide |
| 74. | 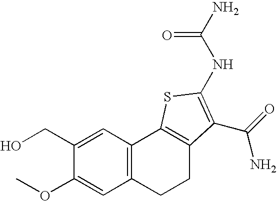 | 2-[(aminocarbonyl)- amino]-8-(hydroxy- methyl)-7-methoxy- 4,5-dihydronaphtho- [1,2-b]-thiophene-3- carboxamide |
| 75. | 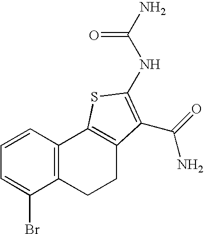 | 2-[(aminocarbonyl)- amino]-6-bromo-4,5- dihydronaphtho[1,2-b]- thiophene-3- carboxamide |
| 76. | 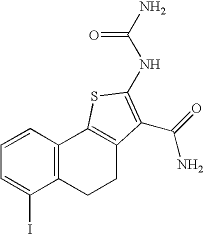 | 2-[(aminocarbonyl)- amino]-6-iodo-4,5- dihydronaphtho[1,2-b]- thiophene-3- carboxamide |
| 77. | 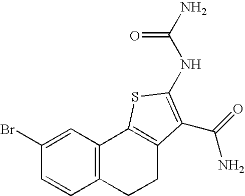 | 2-[(aminocarbonyl)- amino]-8-bromo-4,5- dihydronaphtho[1,2-b]- thiophene-3- carboxamide |
| 78. | 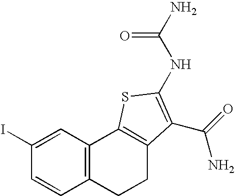 | 2-[(aminocarbonyl)- amino]-8-iodo-4,5- dihydronaphtho[1,2-b]- thiophene-3- carboxamide |
| 79. | 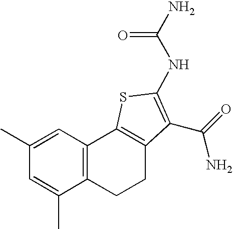 | 2-[(aminocarbonyl)- amino]-6,8-dimethyl- 4,5-dihydronaphtho- [1,2-b]thiophene-3- carboxamide |
| 80. | 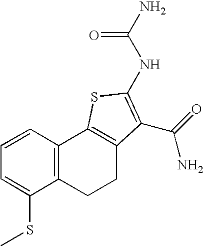 | 2-[(aminocarbonyl)- amino]-6-(methylthio)- 4,5-dihydronaphtho- [1,2-b]thiophene-3- carboxamide |
| 81. | 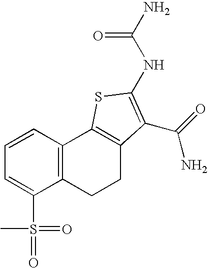 | 2-[(aminocarbonyl)- amino]-6-(methyl- sulfonyl)-4,5-dihydro- naphtho[1,2-b]- thiophene-3- carboxamide |
| 82. | 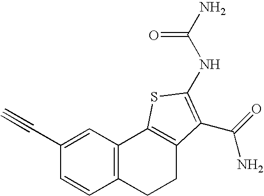 | 2-[(aminocarbonyl)- amino]-8-ethynyl-4,5- dihydronaphtho[1,2-b]- thiophene-3- carboxamide |
| 83. | 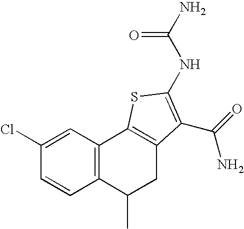 | 2-[(aminocarbonyl)- amino]-8-chloro-5- methyl-4,5-dihydro- naphtho[1,2-b]- thiophene-3- carboxamide |
| 84. | 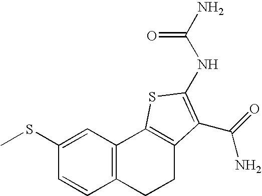 | 2-[(aminocarbonyl)- amino]-8-(methylthio)- 4,5-dihydronaphtho- [1,2-b]thiophene-3- carboxamide |
| 85. | 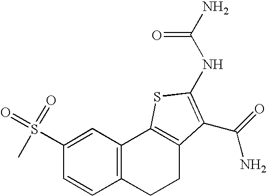 | 2-[(aminocarbonyl)- amino]-8-(methyl- sulfonyl)-4,5-dihydro- naphtho[1,2-b]- thiophene-3- carboxamide |
| 86. | 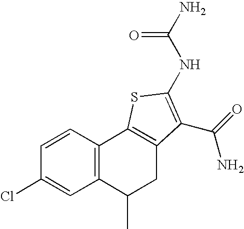 | 2-[(aminocarbonyl)- amino]-7-chloro-5- methyl-4,5-dihydro- naphtho[1,2-b]- thiophene-3- carboxamide |
| 87. | 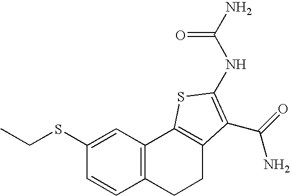 | 2-[(aminocarbonyl)- amino]-8-(ethylthio)- 4,5-dihydronaphtho- [1,2-b]thiophene-3- carboxamide |
| 88. | 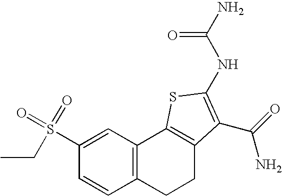 | 2-[(aminocarbonyl)- amino]-8-(ethyl- sulfonyl)-4,5-dihydro- naphtho[1,2-b]- thiophene-3- carboxamide |
| 89. | 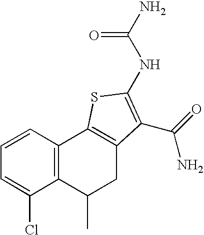 | 2-[(aminocarbonyl)- amino]-6-chloro-5- methyl-4,5-dihydro- naphtho[1,2-b]- thiophene-3- carboxamide |
| 90. | 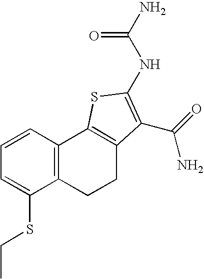 | 2-[(aminocarbonyl)- amino]-6-(ethylthio)- 4,5-dihydronaphtho- [1,2-b]thiophene-3- carboxamide |
| 91. | 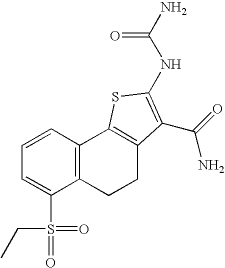 | 2-[(aminocarbonyl)- amino]-6-(ethyl- sulfonyl)-4,5-dihydro- naphtho[1,2-b]- thiophene-3- carboxamide |
| 92. | 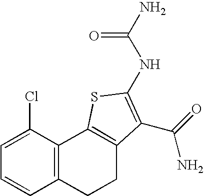 | 2-[(aminocarbonyl)- amino]-9-chloro-4,5- dihydronaphtho[1,2-b]- thiophene-3- carboxamide |
| 93. | 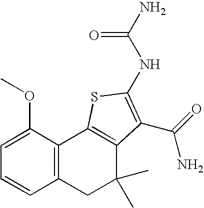 | 2-[(aminocarbonyl)- amino]-9-methoxy-4,4- dimethyl-4,5-dihydro- naphtho[1,2-b-]- thiophene-3- carboxamide |
| 94. | 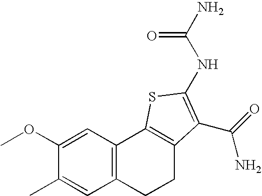 | 2-[(aminocarbonyl)- amino]-8-methoxy-7- methyl-4,5-dihydron- aphtho[1,2-b]- thiophene-3- carboxamide |
| 95. | 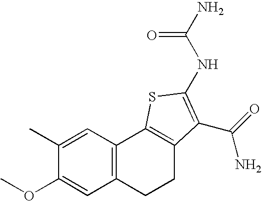 | 2-[(aminocarbonyl)- amino]-7-methoxy-8- methyl-4,5-dihydro- naphtho[1,2-b]- thiophene-3- carboxamide |
| 96. | 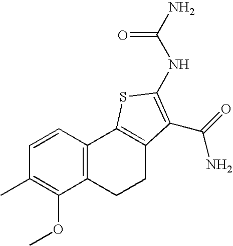 | 2-[(aminocarbonyl)- amino]-6-methoxy-7- methyl-4,5-dihydro- naphtho[1,2-b]- thiophene-3- carboxamide |
| 97. | 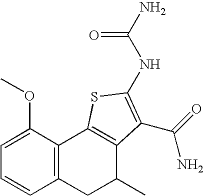 | 2-[(aminocarbonyl)- amino]-9-methoxy-4- methyl-4,5-dibydro- naphtho[1,2-b]- thiophene-3- carboxamide |
| 98. | 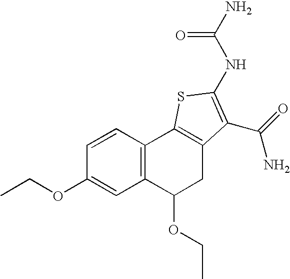 | 2-[(aminocarbonyl)- amino]-6,7-diethoxy- 4,5-dihydronaphtho[- 1,2-b]thiophene-3- carboxamide |
| 99. | 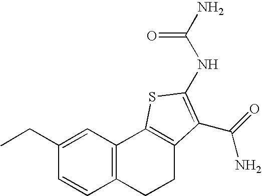 | 2-[(aminocarbonyl)- amino]-8-ethyl-4,5- dihydronaphtho[1,2-b]- thiophene-3- carboxamide |
| 100. | 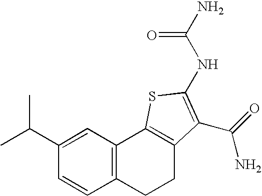 | 2-[(aminocarbonyl)- amino]-8-isopropyl- 4,5-dihydronaphtho- [1,2-b]-thiophene-3- carboxamide |
| 101. | 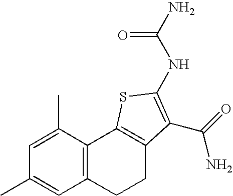 | 2-[(aminocarbonyl)- amino]-7,9-dimethyl- 4,5-dihydronaphtho- [1,2-b]thiophene-3- carboxamide |
| 102. | 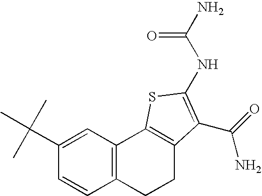 | 2-[(aminocarbonyl)- amino]-8-tert-butyl- 4,5-dihydronaphtho- [1,2-b]-thiophene-3- carboxamide |
| 103. | 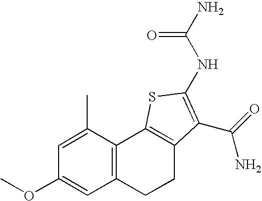 | 2-[(aminocarbonyl)- amino]-7-methoxy-9- methyl-4,5-dihydro- naphtho[1,2-b]- thiophene-3- carboxamide |
| 104. | 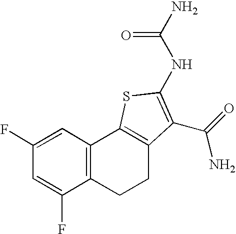 | 2-[(aminocarbonyl)- amino]-6,8-difluoro- 4,5-dihydronaphtho- [1,2-b]-thiophene-3- carboxamide |
| 105. | 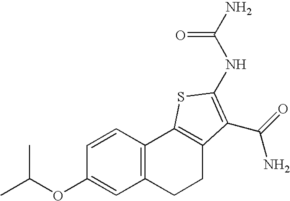 | 2-[(aminocarbonyl)- amino]-7-isopropoxy- 4,5-dihydronaphtho- [1,2-b]thiophene-3- carboxamide |
| 106. | 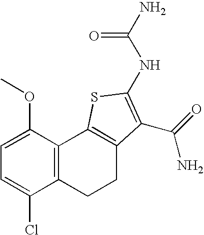 | 2-[(aminocarbonyl)- amino]-6-chloro-9- methoxy-4,5-dihydro- naphtho[1,2-b]- thiophene-3- carboxamide |
| 107. | 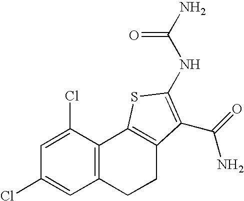 | 2-[(aminocarbonyl)- amino]-7,9-dichloro- 4,5-dihydronaphtho- [1,2-b]thiophene-3- carboxamide |
| 108. | 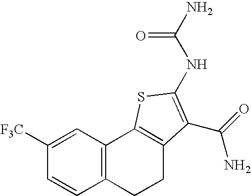 | 2-[(aminocarbonyl)- amino]-8-(trifluoro- methyl)-4,5-dihydro- naphtho[1,2-b]- thiophene-3- carboxamide |
| 109. | 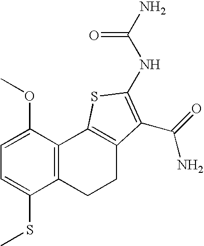 | 2-[(aminocarbonyl)- amino]-9-methoxy-6- (methylthio)-4,5- dihydronaphtho[1,2-b]- thiophene-3- carboxamide |
| 110. | 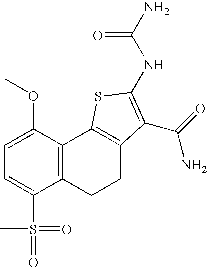 | 2-[(aminocarbonyl)- amino]-9-methoxy-6- (methylsulfonyl)-4,5- dihydronaphtho[1,2-b]- thiophene-3- carboxamide |
| 111. | 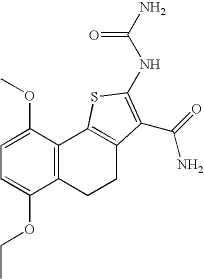 | 2-[(aminocarbonyl)- amino]-6-ethoxy-9- methoxy-4,5-dihydro- naphtho[1,2-b]- thiophene-3- carboxamide |
| 112. | 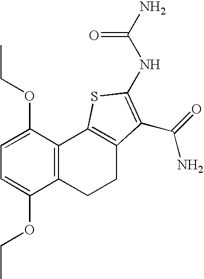 | 2-[(aminocarbonyl)- amino]-6,9-diethoxy- 4,5-dihydronaphtho- [1,2-b]thiophene-3- carboxamide |
| 113. | 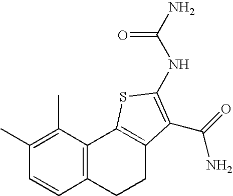 | 2-[(aminocarbonyl)- amino]-8,9-dimethyl- 4,5-dihydronaphtho- [1,2-b]thiophene-3- carboxamide |
| 114. | 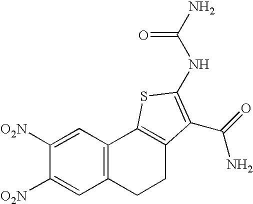 | 2-[(aminocarbonyl)- amino]-7,8-dinitro-4,5- dihydronaphtho[1,2-b]- thiophene-3- carboxamide |
| 115. | 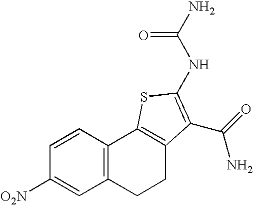 | 2-[(aminocarbonyl)- amino]-7-nitro-4,5- dihydronaphtho[1,2-b]- thiophene-3- carboxamide |
| 116. | 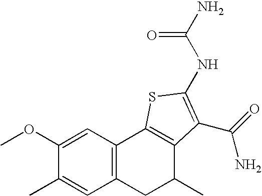 | 2-[(aminocarbonyl)- amino]-8-methoxy-4,7- dimethyl-4,5-dihydro- naphtho[1,2-b]- thiophene-3- carboxamide |
| 117. | 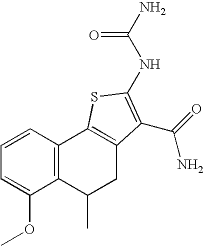 | 2-[(aminocarbonyl)- amino]-6-methoxy-5- methyl-4,5-dihydro- naphtho[1,2-b]- thiophene-3- carboxamide |
| 118. | 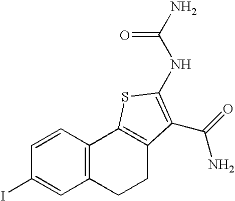 | 2-[(aminocarbonyl)- amino]-7-iodo-4,5- dihydronaphtho[1,2-b]- thiophene-3- carboxamide |
| 119. | 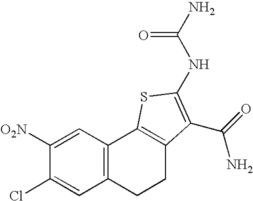 | 2-[(aminocarbonyl)- amino]-7-chloro-8- nitro-4,5-dihydro- naphtho[1,2-b]- thiophene-3- carboxamide |
| 120. | 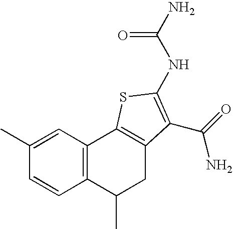 | 2-[(aminocarbonyl)- amino]-5,8-dimethyl- 4,5-dihydronaphtho- [1,2-b]thiophene-3- carboxamide |
| 121. | 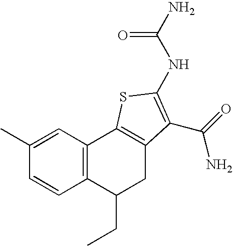 | 2-[(aminocarbonyl)- amino]-5-ethyl-8- methyl-4,5-dihydro- naphtho[1,2-b]- thiophene-3- carboxamide |
| 122. | 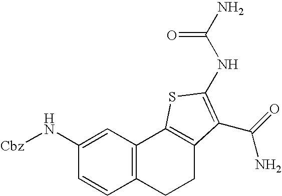 | benzyl 3-(amino- carbonyl)-2-[(amino- carbonyl)amino]-4,5- dihydronaphtho[1,2-b]- thien-8-ylcarbamate |
| 123. | 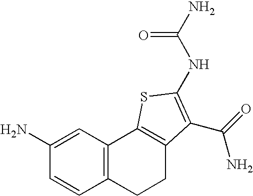 | 8-amino-2-[(amino- carbonyl)amino]-4,5- dihydronapbtho[1,2-b]- thiophene-3- carboxamide |
| 124. | 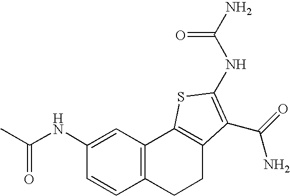 | 8-(acetylamino)-2- [(aminocarbonyl)amino]- 4,5-dihydronaphtho[1,2- b]thiophene-3- carboxamide |
| 125. | 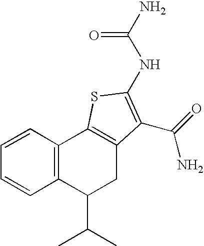 | 2-[(aminocarbonyl)- amino]-5-isopropyl-4,5- dihydronaphtho[1,2-b]- thiophene-3- carboxamide |
| 126. | 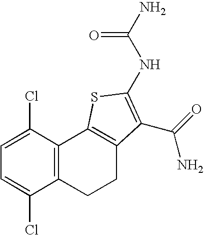 | 2-[(aminocarbonyl)- amino]-6,9-dichloro-4,5- dihydronaphtho[1,2-b]- thiophene-3- carboxamide |
| 127. | 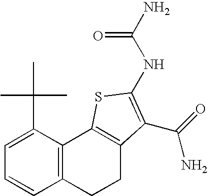 | 2-[(aminocarbonyl)- amino]-9-tert-butyl-4,5- dihydronaphtho[1,2-b]- thiophene-3- carboxamide |
| 128. | 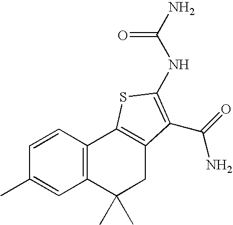 | 2-[(aminocarbonyl)amino]- 5,5,7-trimethyl-4,5- dihydronaphtho[1,2-b]- thiophene-3- carboxamide |
| 129. | 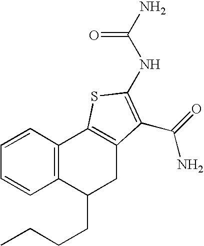 | 2-[(aminocarbonyl)- amino]-5-butyl-4,5- dihydronaphtho[1,2-b]- thiophene-3- carboxamide |
| 130. | 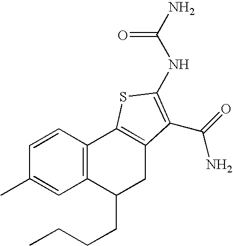 | 2-[(aminocarbonyl)- amino]-5-butyl-7- methyl-4,5-dihydro- naphtho[1,2-b]- thiophene-3- carboxamide |
| 131. | 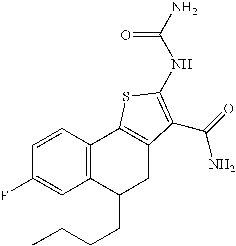 | 2-[(aminocarbonyl)- amino]-5-butyl-7-fluoro- 4,5-dihydronaphtho[1,2- b]thiophene-3- carboxamide |
| 132. | 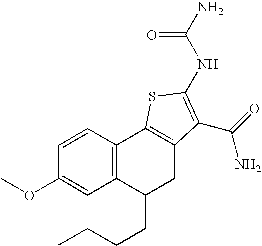 | 2-[(aminocarbonyl)- amino]-5-butyl-7- methoxy-4,5-dihydro- naphtho[1,2-b]- thiophene-3- carboxamide |
| 133. | 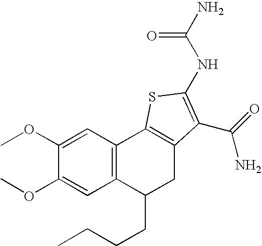 | 2-[(aminocarbonyl)- amino]-5-butyl-7,8- dimethoxy-4,5- dihydronaphtho[1,2-b]- thiophene-3- carboxamide |
| 134. | 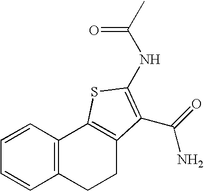 | 2-(acetylamino)-4,5- dihydronaphtho[1,2-b]- thiophene-3- carboxamide |
| 135. | 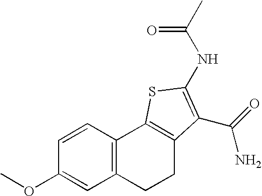 | 2-(acetylamino)-7- methoxy-4,5-dihydro- naphtho[1,2-b]- thiophene-3- carboxamide |
| 136. | 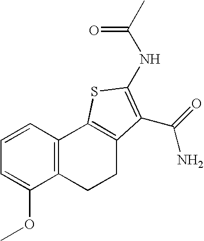 | 2-(acetylamino)-6- methoxy-4,5-dihydro- naphtho[1,2-b]- thiophene-3- carboxamide |
| 137. | 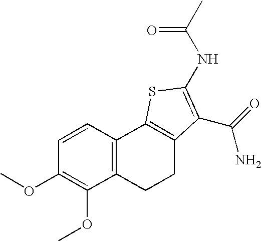 | 2-(acetylamino)-6,7- dimethoxy-4,5- dihydronaphtho[1,2-b]- thiophene-3- carboxamide |
| 138. | 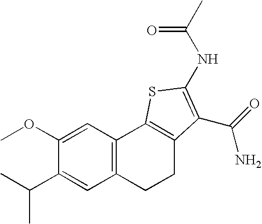 | 2-(acetylamino)-7- isopropyl-8-methoxy- 4,5-dihydronaphtho- [1,2-b]thiophene-3- carboxamide |
| 139. | 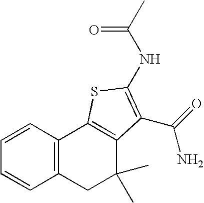 | 2-(acetylamino)-4,4- dimethyl-4,5-dihydro- naphtho[1,2-b]- thiophene-3- carboxamide |
| 140. | 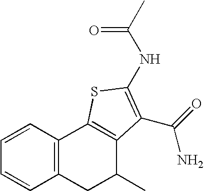 | 2-(acetylamino)-4- methyl-4,5-dihydro- naphtho[1,2-b]- thiophene-3- carboxamide |
| 141. | 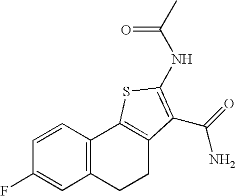 | 2-(acetylamino)-7- fluoro-4,5-dihydro- naphtho[1,2-b]- thiophene-3- carboxamide |
| 142. | 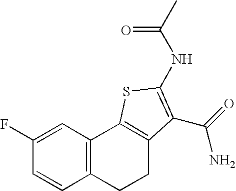 | 2-(acetylamino)-8- fluoro-4,5-dihydro- naphtho[1,2-b]- thiophene-3- carboxamide |
| 143. | 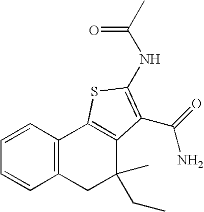 | 2-(acetylamino)-4- ethyl-4-methyl-4,5- dihydronaphtho[1,2-b]- thiophene-3- carboxamide |
| 144. | 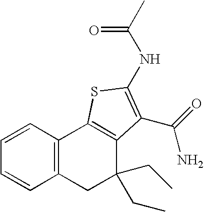 | 2-(acetylamino)-4,4- diethyl-4,5- dihydronaphtho[1,2-b]- thiophene-3- carboxamide |
| 145. | 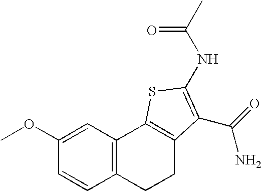 | 2-(acetylamino)-8- methoxy-4,5-dihydro- naphtho[1,2-b]- thiophene-3- carboxamide |
| 146. | 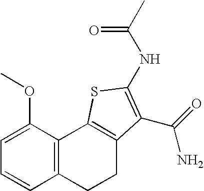 | 2-(acetylamino)-9- methoxy-4,5-dihydro- naphtho[1,2-b]- thiophene-3- carboxamide |
| 147. | 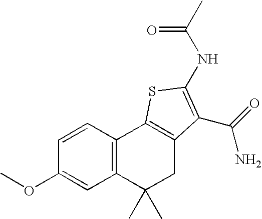 | 2-(acetylamino)-7- methoxy-5,5-dimethyl- 4,5-dihydronaphtho- [1,2-b]thiophene-3- carboxamide |
| 148. | 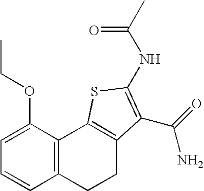 | 2-(acetylamino)-9- ethoxy-4,5-dihydro- naphtho[1,2-b]- thiophene-3- carboxamide |
| 149. | 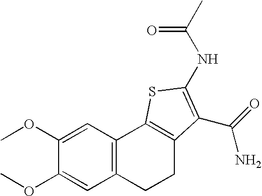 | 2-(acetylamino)-7,8- dimethoxy-4,5- dihydronaphtho[1,2-b]- thiophene-3- carboxamide |
| 150. | 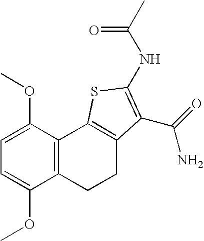 | 2-(acetylamino)-6,9- dimethoxy-4,5- dihydronaphtho[1,2-b]- thiophene-3- carboxamide |
| 151. | 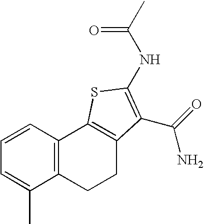 | 2-(acetylamino)-6- methyl-4,5-dihydro- naphtho[1,2-b]- thiophene-3- carboxamide |
| 152. | 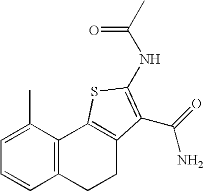 | 2-(acetylamino)-9- methyl-4,5-dihydro naphtho[1,2-b]- thiophene-3- carboxamide |
| 153. | 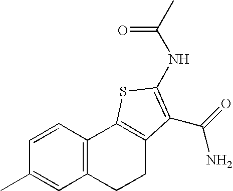 | 2-(acetylamino)-7- methyl-4,5-dihydro- naphtho[1,2-b]- thiophene-3- carboxamide |
| 154. | 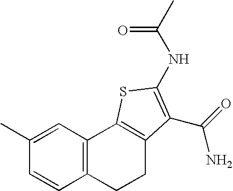 | 2-(acetylamino)-8- methyl-4,5-dihydro- naphtho[1,2-b]- thiophene-3- carboxamide |
| 155. | 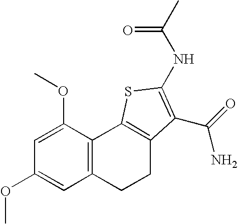 | 2-(acetylamino)-7,9- dimethoxy-4,5- dihydro-naphtho[1,2- b]-thiophene-3- carboxamide |
| 156. | 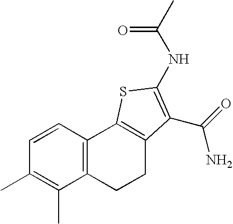 | 2-(acetylamino)-6,7- dimethyl-4,5-dihydro- naphtho[1,2-b]- thiophene-3- carboxamide |
| 157. | 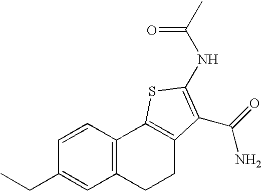 | 2-(acetylamino)-7- ethyl-4,5-dihydro- naphtho[1,2-b]- thiophene-3- carboxamide |
| 158. | 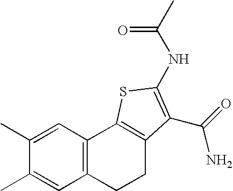 | 2-(acetylamino)-7,8- dimethyl-4,5-dihydro- naphtho[1,2-b]- thiophene-3- carboxamide |
| 159. | 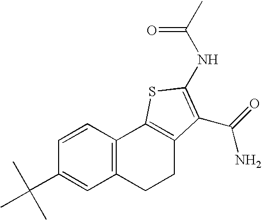 | 2-(acetylamino)-7-tert- butyl-4,5-dihydro- naphtho[1,2-b]- thiophene-3- carboxamide |
| 160. | 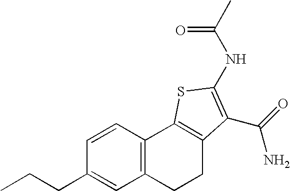 | 2-(acetylamino)-7- propyl-4,5-dihydro- naphtho[1,2-b]- thiophene-3- carboxamide |
| 161. | 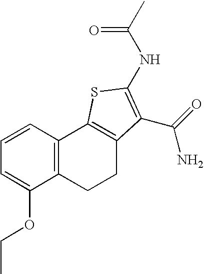 | 2-(acetylamino)-6- ethoxy-4,5-dihydro- naphtho[1,2-b]- thiophene-3- carboxamide |
| 162. | 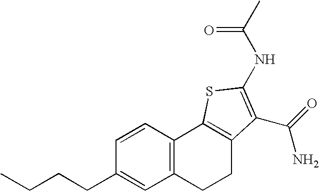 | 2-(acetylamino)-7- butyl-4,5-dihydro- naphtho[1,2-b]- thiophene-3- carboxamide |
| 163. | 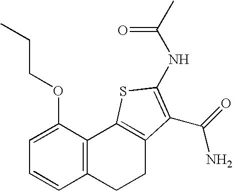 | 2-(acetylamino)-9- propoxy-4,5-dihydro- naphtho[1,2-b]- thiophene-3- carboxamide |
| 164. | 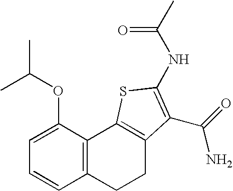 | 2-(acetylamino)-9- isopropoxy-4,5- dihydronaphtho[1,2-b]- thiophene-3- carboxamide |
| 165. | 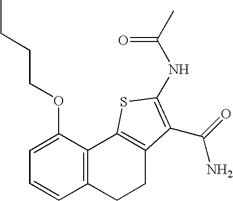 | 2-(acetylamino)-9- butoxy-4,5-dihydro- naphtho[1,2-b]- thiophene-3- carboxamide |
| 166. | 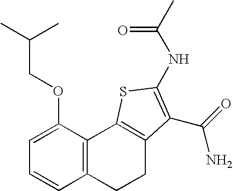 | 2-(acetylamino)-9- isobutoxy-4,5-dihydro- naphtho[1,2-b]- thiophene-3- carboxamide |
| 167. | 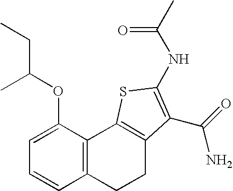 | 2-(acetylamino)-9-sec- butoxy-4,5-dihydro- naphtho[1,2-b]- thiophene-3- carboxamide |
| 168. | 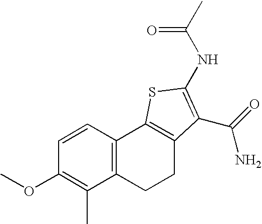 | 2-(acetylamino)-7- methoxy-6-methyl-4,5- dihydronaphtho[1,2-b]- thiophene-3- carboxamide |
| 169. | 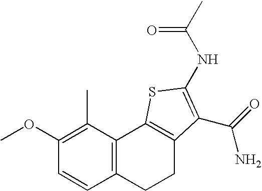 | 2-(acetylamino)-8- methoxy-9-methyl-4,5- dihydronaphtho[1 ,2-b]- thiophene-3- carboxamide |
| 170. | 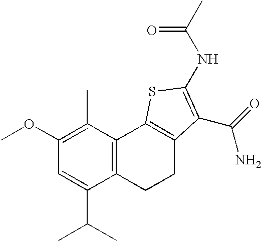 | 2-(acetylamino)-6- isopropyl-8-methoxy- 9-methyl-4,5-dihydro- naphtho[1,2-b]- thiophene-3- carboxamide |
| 171. | 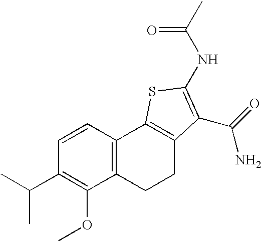 | 2-(acetylamino)-7- isopropyl-6-methoxy- 4,5-dihydronaphtho- [1,2-b]thiophene-3- carboxamide |
| 172. | 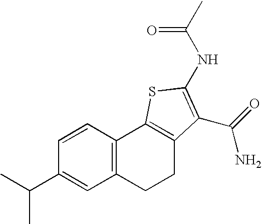 | 2-(acetylamino)-7- isopropyl-4,5-dihydro- naphtho[1,2-b]- thiophene-3- carboxamide |
| 173. | 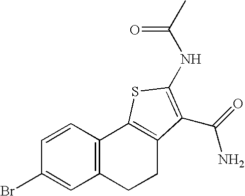 | 2-(acetylamino)-7- bromo-4,5-dihydro- naphtho[1,2-b]- thiophene-3- carboxamide |
| 174. | 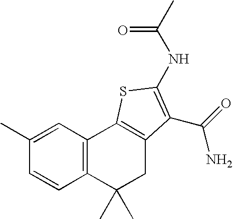 | 2-(acetylamino)-5,5,8- trimethyl-4,5-dihydro- naphtho[1,2-b]- thiophene-3- carboxamide |
| 175. | 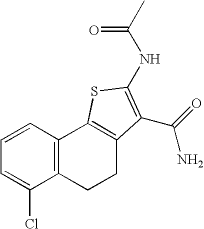 | 2-(acetylamino)-6- chloro-4,5-dihydro- naphtho[1,2-b]- thiophene-3- carboxamide |
| 176. | 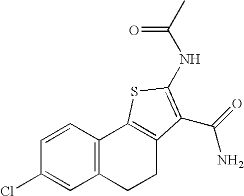 | 2-(acetylamino)-7- chloro-4,5-dihydro- naphtho[1,2-b]- thiophene-3- carboxamide |
| 177. | 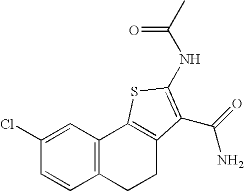 | 2-(acetylamino)-8- chloro-4,5-dihydro- naphtho-[1,2- b]thiophene-3- carboxamide |
| 178. | 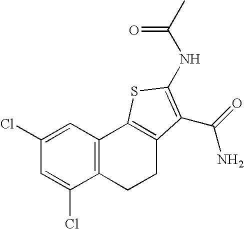 | 2-(acetylamino)-6,8- dichloro-4,5-dihydro- naphtho[1,2-b]- hiophene-3- carboxamide |
| 179. | 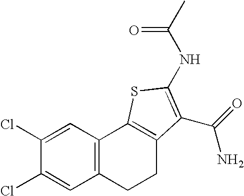 | 2-(acetylamino)-7,8- dichloro-4,5-dihydro- naphtho[1,2-b]- thiophene-3- carboxamide |
| 180. | 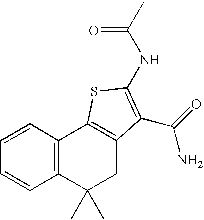 | 2-(acetylamino)-5,5- dimethyl-4,5-dihydro- naphtho[1,2-b]- thiophene-3- carboxamide |
| 181. | 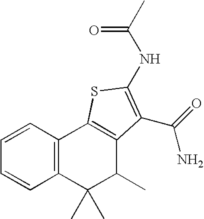 | 2-(acetylamino)-4,5,5- trimethyl-4,5-dihydro- naphtho[1,2-b]- thiophene-3- carboxamide |
| 182. | 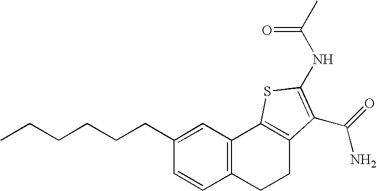 | 2-(acetylamino)-8- hexyl-4,5-dihydro- naphtho[1,2-b]- thiophene-3- carboxamide |
| 183. | 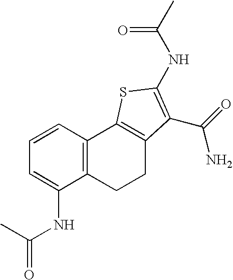 | 2,6-bis(acetylamino)- 4,5-dihydronaphtho- [1,2-b]thiophene-3- carboxamide |
| 184. | 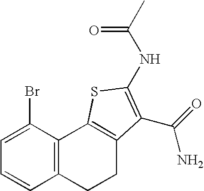 | 2-(acetylamino)-9- bromo-4,5-dihydro- naphtho[1,2-b]- thiophene-3- carboxamide |
| 185. | 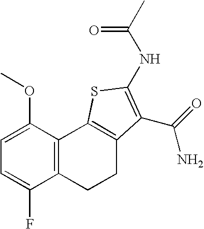 | 2-(acetylamino)-6- fluoro-9-methoxy-4,5- dihydronaphtho[1,2-b]- thiophene-3- carboxamide |
| 186. | 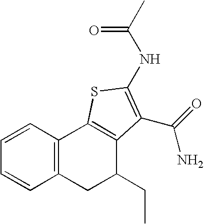 | 2-(acetylamino)-4- ethyl-4,5-dihydro- naphtho[1,2-b]- thiophene-3- carboxamide |
| 187. | 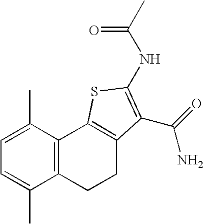 | 2-(acetylamino)-6,9- dimethyl-4,5-dihydro- naphtho[1,2-b]- thiophene-3- carboxamide |
| 188. | 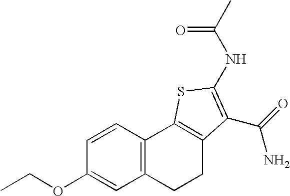 | 2-(acetylamino)-7- ethoxy-4,5-dihydro- naphtho[1,2-b]- thiophene-3- carboxamide |
| 189. | 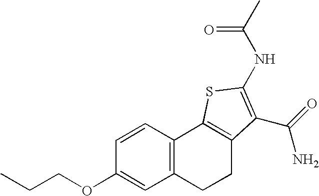 | 2-(acetylamino)-7- propoxy-4,5-dihydro- naphtho[1,2-b]- thiophene-3- carboxamide |
| 190. | 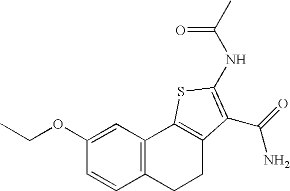 | 2-(acetylamino)-8- ethoxy-4,5-dihydro- naphtho[1,2-b]- thiophene-3- carboxamide |
| 191. | 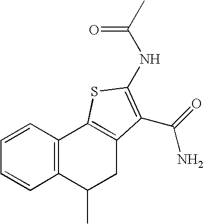 | 2-(acetylamino)-5- methyl-4,5-dihydro- naphtho[1,2-b]- thiophene-3- carboxamide |
| 192. | 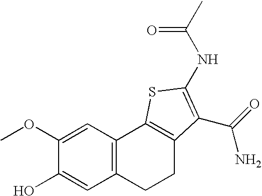 | 2-(acetylamino)-7- hydroxy-8-methoxy- 4,5-dihydronaphtho- [1,2-b]thiophene-3- carboxamide |
| 193. | 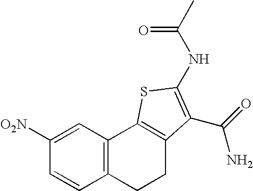 | 2-(acetylamino)-8- nitro-4,5-dihydro- naphtho[1,2-b]- thiophene-3- carboxamide |
| 194. | 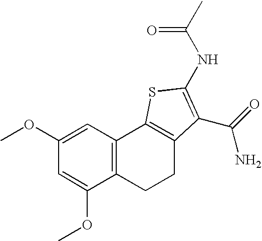 | 2-(acetylamino)-6,8- dimethoxy-4,5- dihydronaphtho[1,2-b]- thiophene-3- carboxamide |
| 195. | 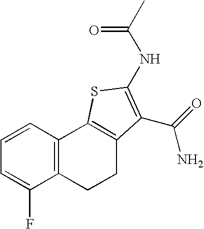 | 2-(acetylamino)-6- fluoro-4,5-dihydro- naphtho[1,2-b]- thiophene-3- carboxamide |
| 196. | 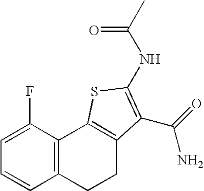 | 2-(acetylamino)-9- fluoro-4,5-dihydro- naphtho[1,2-b]- thiophene-3- carboxamide |
| 197. | 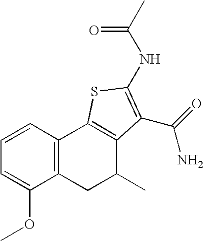 | 2-(acetylamino)-6- methoxy-4-methyl-4,5- dihydronaphtho[1,2-b]- thiophene-3- carboxamide |
| 198. | 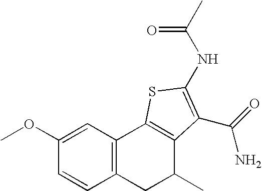 | 2-(acetylamino)-8- methoxy-4-methyl-4,5- dihydronaphtho[1,2-b]- thiophene-3- carboxamide |
| 199. | 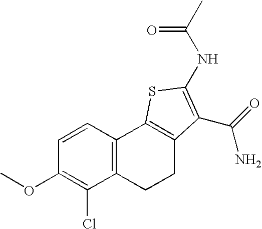 | 2-(acetylamino)-6- chloro-7-methoxy-4,5- dihydronaphtho[1,2-b]- thiophene-3- carboxamide |
| 200. | 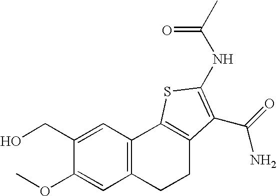 | 2-(acetylamino)-8- methoxy-4,5-dihydro- naphtho[1,2-b]- thiophene-3- carboxamide |
| 201. | 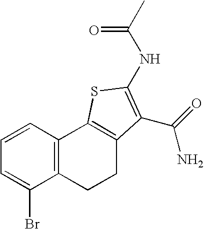 | 2-(acetylamino)-6- bromo-4,5-dihydro- naphtho[1,2-b]- thiophene-3- carboxamide |
| 202. | 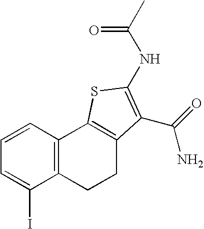 | 2-(acetylamino)-6- iodo-4,5-dihydro- naphtho-[1,2- b]thiophene-3- carboxamide |
| 203. | 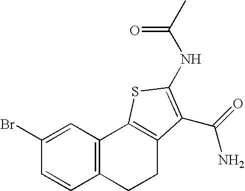 | 2-(acetylamino)-8- bromo-4,5-dihydro- naphtho[1,2-b]- thiophene-3- carboxamide |
| 204. | 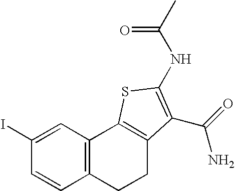 | 2-(acetylamino)-8- iodo-4,5-dihydro- naphtho-[1,2-b]- thiophene-3- carboxamide |
| 205. | 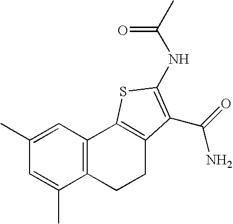 | 2-(acetylamino)-6,8- dimethyl-4,5-dihydro- naphtho[1,2-b]- thiophene-3- carboxamide |
| 206. | 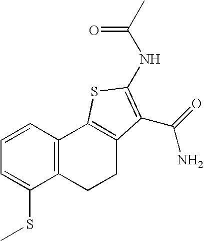 | 2-(acetylamino)-6- (methylthio)-4,5- dihydronaphtho[1,2-b]- thiophene-3- carboxamide |
| 207. | 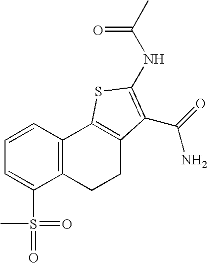 | 2-(acetylamino)-6- (methylsulfonyl)-4,5- dihydronaphtho[1,2-b]- thiophene-3- carboxamide |
| 208. | 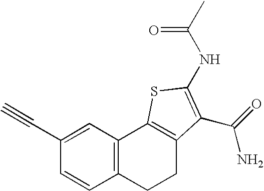 | 2-(acetylamino)-8- ethynyl-4,5-dihydro- naphtho[1,2-b]- thiophene-3- carboxamide |
| 209. | 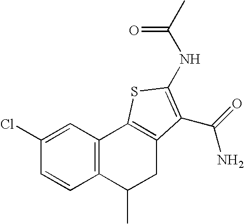 | 2-(acetylamino)-8- chloro-5-methyl-4,5- dihydronaphtho[1,2-b]- thiophene-3- carboxamide |
| 210. | 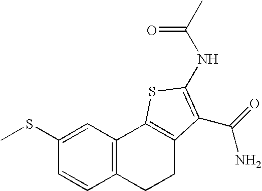 | 2-(acetylamino)-8- (methylthio)-4,5- dihydronaphtho[1,2-b]- thiophene-3- carboxamide |
| 211. | 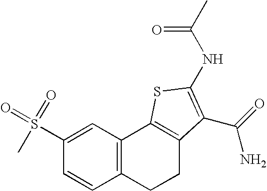 | 2-(acetylamino)-8- (methylsulfonyl)-4,5- dihydronaphtho[1,2-b]- thiophene-3- carboxamide |
| 212. | 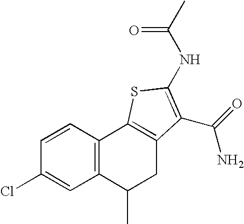 | 2-(acetylamino)-7- chloro-5-methyl-4,5- dihydronaphtho[1,2-b]- thiophene-3- carboxamide |
| 213. | 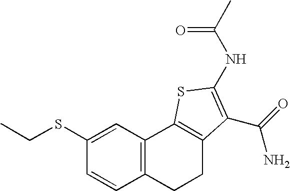 | 2-(acetylamino)-8- (ethylthio)-4,5- dihydro-naphtho[1,2b]- thiophene-3- carboxamide |
| 214. | 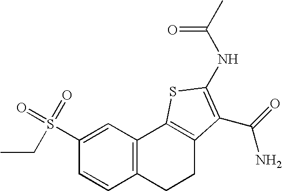 | 2-(acetylamino)-8- (ethylsulfonyl)-4,5- dihydronaphtho[1,2-b]- thiophene-3- carboxamide |
| 215. | 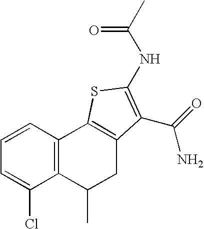 | 2-(acetylamino)-6- chloro-5-methyl-4,5- dihydronaphtho[1,2-b]- thiophene-3- carboxamide |
| 216. | 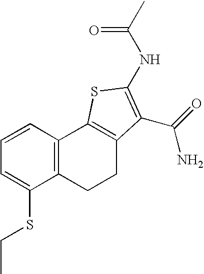 | 2-(acetylamino)-6- (ethylthio)-4,5- dihydronaphtho[1,2-b]- thiophene-3- carboxamide |
| 217. | 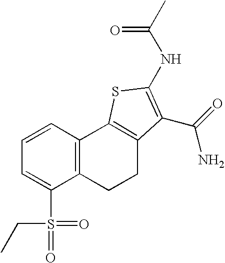 | 2-(acetylamino)-6- (ethylsulfonyl)-4,5- dihydronaphtho[1,2-b]- thiophene-3- carboxamide |
| 218. | 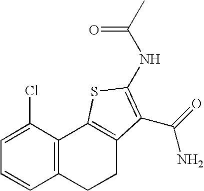 | 2-(acetylamino)-9- chloro-4,5-dihydro- naphtho[1,2-b]- thiophene-3- carboxamide |
| 219. | 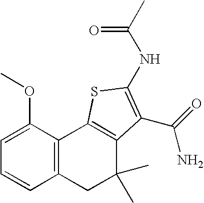 | 2-(acetylamino)-9- methoxy-4,4-dimethyl- 4,5-dihydronaphtho- [1,2-b]thiophene-3- carboxamide |
| 220. | 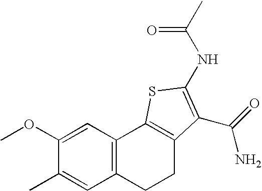 | 2-(acetylamino)-8- methoxy-7-methyl-4,5- dihydronaphtho[1,2-b]- thiophene-3- carboxamide |
| 221. | 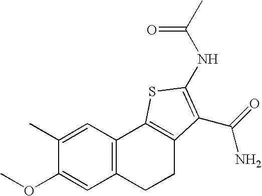 | 2-(acetylamino)-7- methoxy-8-methyl-4,5- dihydronaphtho[1,2-b]- thiophene-3- carboxamide |
| 222. | 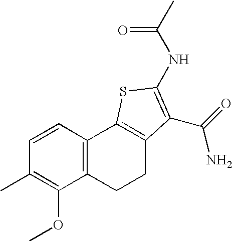 | 2-(acetylamino)-6- methoxy-7-methyl-4,5- dihydronaphtho[1,2-b]- thiophene-3- carboxamide |
| 223. | 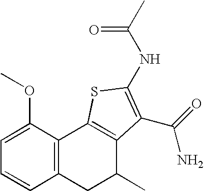 | 2-(acetylamino)-9- methoxy-4-methyl-4,5- dihydronaphtho[1,2-b]- thiophene-3- carboxamide |
| 224. | 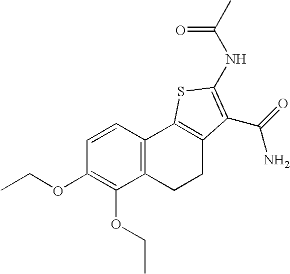 | 2-(acetylamino)-6,7- diethoxy-4,5-dihydro- naphtho[1,2-b]- thiophene-3- carboxamide |
| 225. | 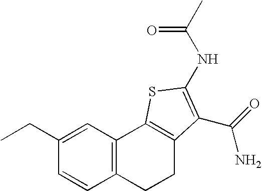 | 2-(acetylamino)-8- ethyl-4,5-dihydro- naphtho[1,2-b]- thiophene-3- carboxamide |
| 226. | 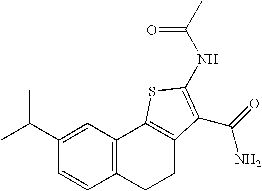 | 2-(acetylamino)-8- isopropyl-4,5-dihydro- naphtho[1,2-b]- thiophene-3- carboxamide |
| 227. | 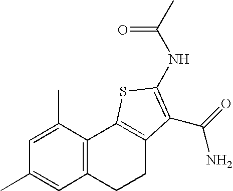 | 2-(acetylamino)-7,9- dimethyl-4,5-dihydro- naphtho[1,2-b]- thiophene-3- carboxamide |
| 228. | 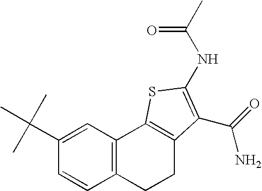 | 2-(acetylamino)-8-tert- butyl-4,5-dihydro- naphtho[1,2-b]- thiophene-3- carboxamide |
| 229. | 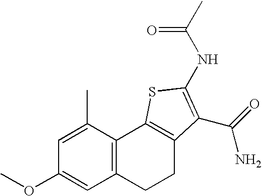 | 2-(acetylamino)-7- methoxy-9-methyl-4,5- dihydronaphtho[1,2-b]- thiophene-3- carboxamide |
| 230. | 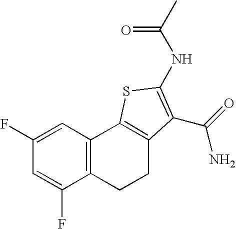 | 2-(acetylamino)-6,8- difluoro-4,5-dihydro- naphtho[1,2-b]- thiophene-3- carboxamide |
| 231. | 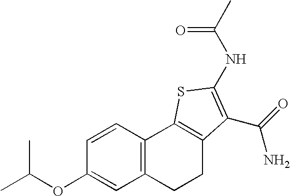 | 2-(acetylamino)-7- isopropoxy-4,5- dihydronaphtho[1,2- b]thiophene-3- carboxamide |
| 232. | 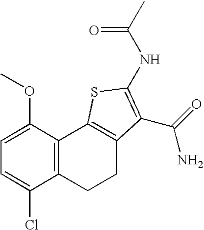 | 2-(acetylamino)-6- chloro-9-methoxy-4,5- dihydronaphtho[1,2- b]-thiophene-3- carboxamide |
| 233. | 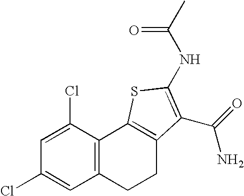 | 2-(acetylamino)-7,9- dichloro-4,5-dihydro- naphtho[1,2-b]- thiophene-3- carboxamide |
| 234. | 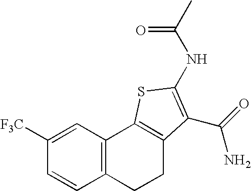 | 2-(acetylamino)-8- (trifluoromethyl)-4,5- dihydronaphtho[1,2-b]thiophene-3- carboxamide |
| 235. | 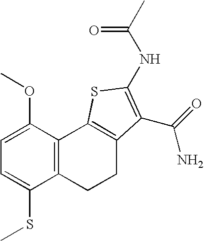 | 2-(acetylamino)-9- methoxy-6- (methylthio)-4,5- dihydronaphtho[1,2-b]- thiophene-3- carboxamide |
| 236. | 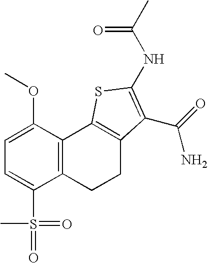 | 2-(acetylamino)-9- methoxy-6-(methyl- sulfonyl)-4,5-dihydro- naphtho[1,2-b]- thiophene-3- carboxamide |
| 237. | 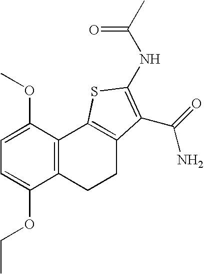 | 2-(acetylamino)-6- ethoxy-9-methoxy- 4,5-dihydronaphtho- [1,2-b]thiophene-3- carboxamide |
| 238. | 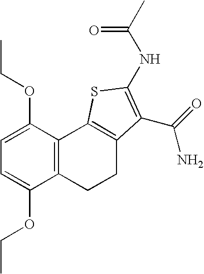 | 2-(acetylamino)-6,9- dlethoxy-4,5-dihydro- thiophene-3- carboxamide |
| 239. | 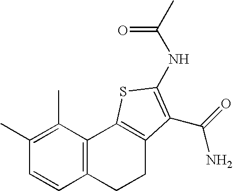 | 2-(acetylamino)-8,9- dimethyl-4,5-dihydro- naphtho[1,2-b]- thiophene-3- carboxamide |
| 240. | 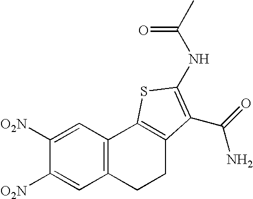 | 2-(acetylamino)-7,8- dinitro-4,5-dihydro- naphtho[1,2-b]- thiophene-3- carboxamide |
| 241. | 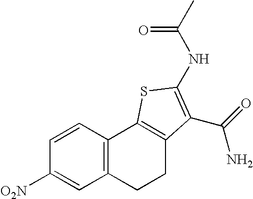 | 2-(acetylamino)-7- nitro-4,5-dihydro- naphtho-[1,2-b]- thiophene-3- carboxamide |
| 242. | 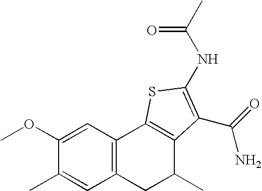 | 2-(acetylamino)-8- methoxy-4,7-dimethyl- 4,5-dihydronaphtho- [1,2-b]thiophene-3- carboxamide |
| 243. | 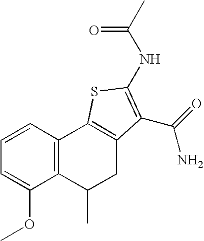 | 2-(acetylamino)-6- methoxy-5-methyl-4,5- dihydronaphtho[1,2-b]- thiophene-3- carboxamide |
| 244. | 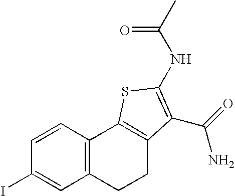 | 2-(acetylamino)-7- iodo-4,5-dihydro- naphtho[1,2-b]- thiophene-3- carboxamide |
| 245. | 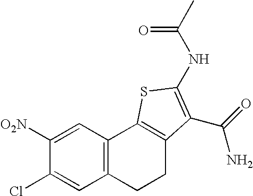 | 2-(acetylamino)-7- chloro-8-nitro-4,5- dihydronaphtho[1,2-b]- thiophene-3- carboxamide |
| 246. | 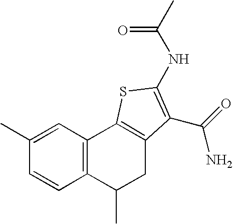 | 2-(acetylamino)-5,8- dimethyl-4,5-dihydro- naphtho[1,2-b]- thiophene-3- carboxamide |
| 247. | 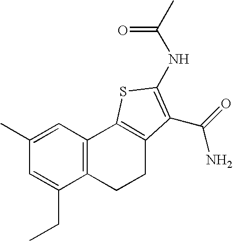 | 2-(acetylamino)-5- ethyl-8-methyl-4,5- dihydronaphtho[1,2-b]- thiophene-3- carboxamide |
| 248. | 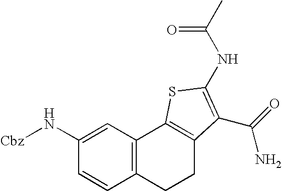 | benzyl 2-(acetyl- amino)-3-(amino- carbonyl)-4,5-dihydro- naphtho[1,2-b]-thien-8- ylcarbamate |
| 249. | 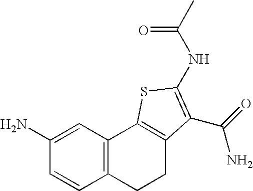 | 2-(acetylamino)-8- amino-4,5-dihydro- naphtho[1,2-b]- thiophene-3- carboxamide |
| 250. | 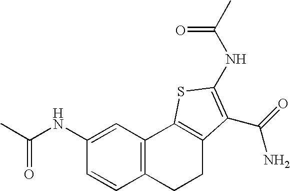 | 2,8-bis(acetylamino)- 4,5-dihydronaphtho- [1,2-b]thiophene-3- carboxamide |
| 251. | 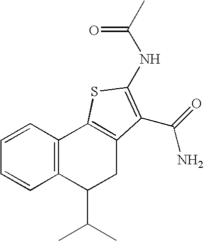 | 2-(acetylamino)-5- isopropyl-4,5-dihydro- naphtho[1,2-b]- thiophene-3- carboxamide |
| 252. | 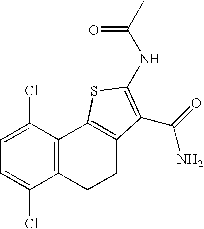 | 2-(acetylamino)-6,9- dichloro-4,5-dihydro- naphtho[1,2-b]- thiophene-3- carboxamide |
| 253. | 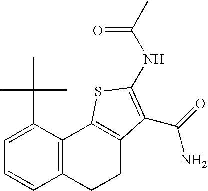 | 2-(acetylamino)-9-tert- butyl-4,5-dihydro- naphtho[1,2-b]- thiophene-3- carboxamide |
| 254. | 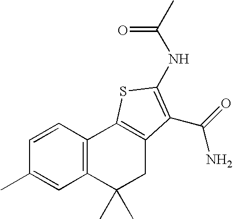 | 2-(acetylamino)-5,5,7- trimethyl-4,5-dihydro- naphtho[1,2-b]- thiophene-3- carboxamide |
| 255. | 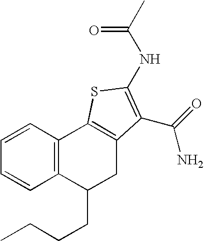 | 2-(acetylamino)-5- butyl-4,5-dihydro- naphtho[1,2-b]- thiophene-3- carboxamide |
| 256. | 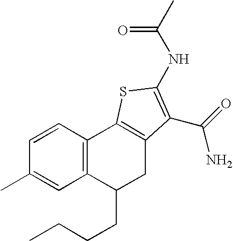 | 2-(acetylamino)-5- butyl-7-methyl-4,5- dihydronaphtho[1,2-b]- thiophene-3- carboxamide |
| 257. | 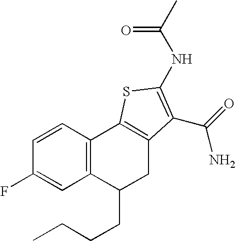 | 2-(acetylamino)-5- butyl-7-fluoro-4,5- dihydronaphtho[1,2-b]- thiophene-3- carboxamide |
| 258. | 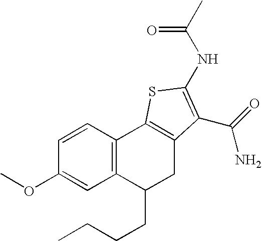 | 2-(acetylamino)-5- butyl-7-methoxy-4,5- dihydronaphtho[1,2-b]- thiophene-3- carboxamide |
| 259. | 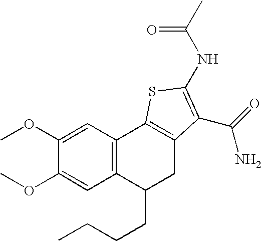 | 2-(acetylamino)-5- butyl-7,8-dimethoxy- 4,5-dihydronaphtho- [1,2-b]thiophene-3- carboxamide |
| 260. | 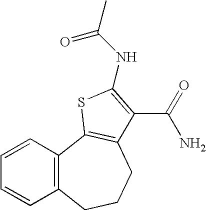 | 2-(acetylamino)-5,6- dihydro-4H-benzo- [6,7]-cyclohepta[1,2- b]thiophene-3- carboxamide |
| 261. | 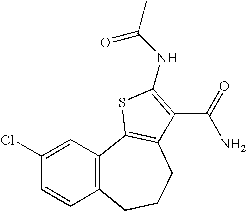 | 2-(acetylamino)-9- chloro-5,6-dihydro-4H- benzo[6,7]cyclo- hepta[1,2-b]thiophene- 3-carboxamide |
| 262. | 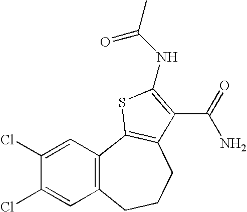 | 2-(acetylamino)-8,9- dichloro-5,6-dihydro-4H- benzo[6,7]cyclohepta[1,2- b]thiophene-3- carboxamide |
| 263. | 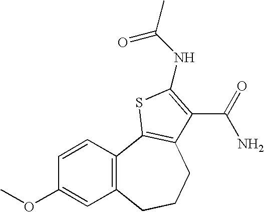 | 2-(acetylamino)-8- methoxy-5,6-dihydro-4H- benzo[6,7]cyclohepta[1,2- b]thiophene-3- carboxamide |
| 264. | 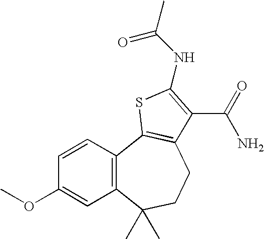 | 2-(acetylamino)-8- methoxy-6,6-dimethyl- 5,6-dihydro-4H-benzo- [6,7]cyclohepta[1,2-b]- thiophene-3-carboxamide |
| 265. | 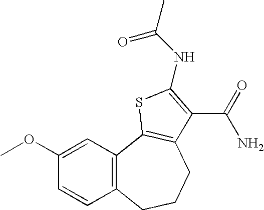 | 2-(acetylamino)-9- methoxy-5,6-dihydro-4H- benzo[6,7]cyclohepta[1,2- b]thiophene-3- carboxamide |
| 266. | 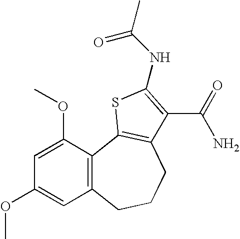 | 2-(acetylamino)-8,10- dimethoxy-5,6-dihydro- 4H-benzo[6,7]cyclo- hepta[1,2-b]thiophene-3- carboxamide |
| 267. | 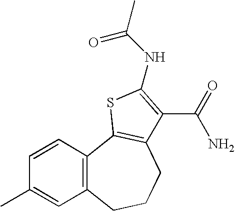 | 2-(acetylamino)-8- methyl-5,6-dihydro-4H- benzo-[6,7]cyclohepta- [1,2-b]-thiophene-3- carboxamide |
| 268. | 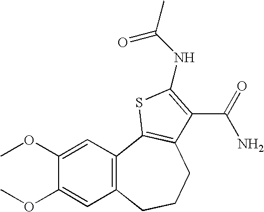 | 2-(acetylamino)-8,9- dimethoxy-5,6-dihydro- 4H-benzo[6,7]cyclo- hepta[1,2-b]thiophene-3- carboxamide |
| 269. | 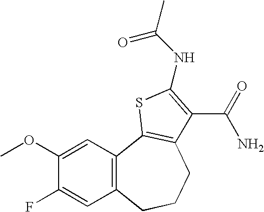 | 2-(acetylamino)-8-fluoro- 9-methoxy-5,6-dihydro- 4H-benzo[6,7]-cyclo- hepta[1,2-b]-thiophene-3- carboxamide |
| 270. | 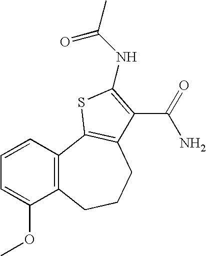 | 2-(acetylamino)-7- methoxy-5,6-dihydro-4H- benzo[6,7]cyclohepta[1,2- b]thiophene-3- carboxamide |
| 271. | 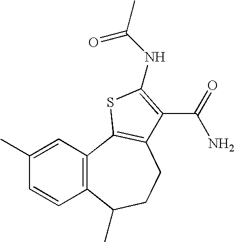 | 2-(acetylamino)-6,9- dimethyl-5,6-dihydro-4H- benzo[6,7]cyclohepta[1,2- b]thiophene-3- carboxamide |
| 272. | 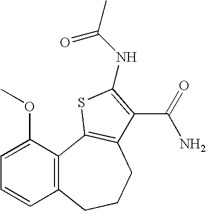 | 2-(acetylamino)-10- methoxy-5,6-dihydro-4H- benzo[6,7]cyclohepta[1,2- b]thiophene-3- carboxamide |
| 273. | 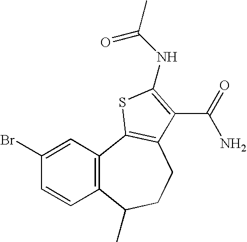 | 2-(acetylamino)-9-bromo- 6-methyl-5,6-dihydro-4H- benzo[6,7]cyclohepta[1,2- b]thiophene-3- carboxamide |
| 274. | 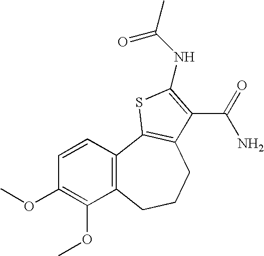 | 2-(acetylamino)-7,8- dimethoxy-5,6-dihydro- 4H-berzo[6,7]cyclo- hepta[1,2-b]thiophene-3- carboxamide |
| 275. | 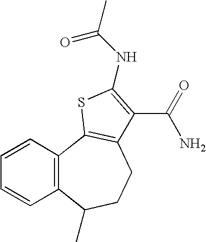 | 2-(acetylamino)-6-methyl- 5,6-dihydro-4H-benzo- [6,7]cyclohepta-[1,2-b]- thiophene-3-carboxamide |
| 276. | 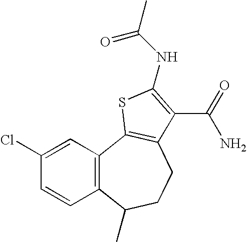 | 2-(acetylamino)-9-chloro- 6-methyl-5,6-dihydro-4H- benzo-[6,7]cyclohepta- [1,2-b]-thiophene-3- carboxamide |
| 277. | 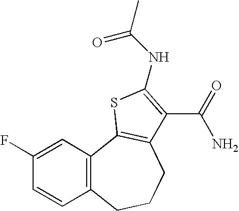 | 2-(acetylamino)-9-fluoro- 5,6-dihydro-4H-benzo- [6,7]cyclohepta[1,2-b]- thiophene-3-carboxamide |
| 278. | 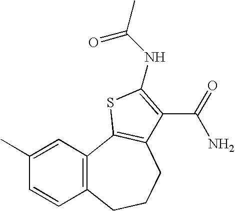 | 2-(acetylamino)-9-methyl- 5,6-dihydro-4H-benzo- [6,7]cyclo-hepta[1,2-b]- thiophene-3-carboxamide |
| 279. | 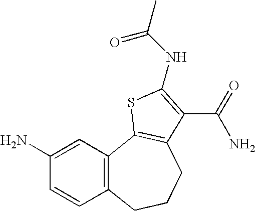 | 2-(acetylamino)-9-amino- 5,6-dihydro-4H-benzo- [6,7]cyclo-hepta[1,2-b]- thiophene-3-carboxamide |
| 280. | 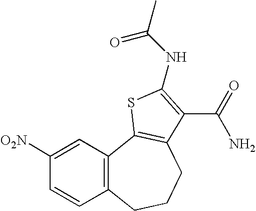 | 2-(acetylamino)-9-nitro- 5,6-dihydro-4H-benzo- [6,7]cyclohepta[1,2-b]- thiophene-3-carboxamide |
| 281. | 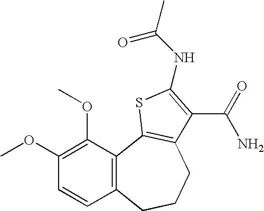 | 2-(acetylamino)-9,10- dimethoxy-5,6-dihydro- 4H-benzo[6,7]cyclo- hepta[1,2-b]thiophene-3- carboxamide |
| 282. | 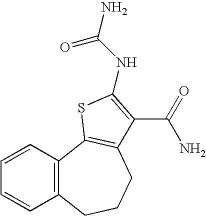 | 2-[(aminocarbonyl)- amino]-5,6-dihydro-4H- benzo[6,7]cyclohepta[1,2- b]thiophene-3- carboxamide |
| 283. | 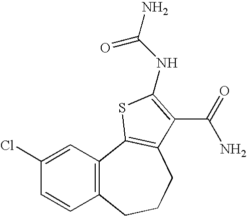 | 2-[(aminocarbonyl)- amino]-9-chloro-5,6- dihydro-4H-benzo[6,7]- cyclohepta[1,2-b]- thiophene-3-carboxamide |
| 284. | 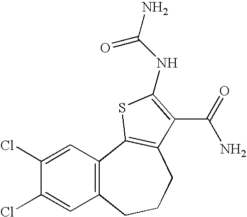 | 2-[(aminocarbonyl)- amino]-8,9-dichloro-5,6- dihydro-4H-benzo[6,7]- cyclohepta[1,2-b]- thiophene-3-carboxamide |
| 285. | 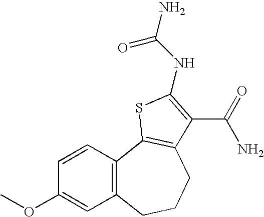 | 2-[(aminocarbonyl)- dihydro-4H-benzo[6,7]- cyclohepta[1,2-b]- thiophene-3-carboxamide |
| 286. | 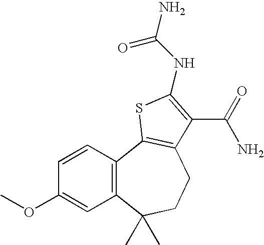 | 2-[(aminocarbonyl)- dimethyl-5,6-dihydro-4H- benzo[6,7]cyclohepta[1,2- b]thiophene-3- carboxamide |
| 287. | 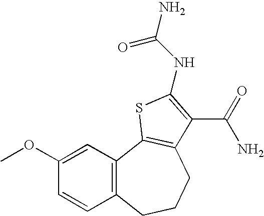 | 2-[(aminocarbonyl)- amino]-9-methoxy-5,6- dihydro-4H-benzo[6,7]- cyclohepta[1,2-b]- thiophene-3-carboxamide |
| 288. | 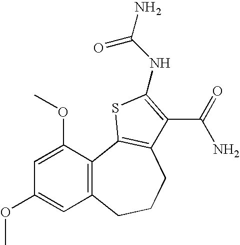 | 2-[(aminocarbonyl)- aminol-8,10-dimethoxy- 5,6-dihydro-4H-benzo- [6,7]cyclohepta[1,2-b]- thiophene-3-carboxamide |
| 289. | 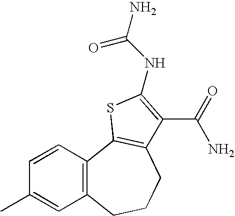 | 2-[(aminocarbonyl)- amino]-8-methyl-5,6- dihydro-4H-benzo[6,7]- cyclohepta[1,2-b]- thiophene-3-carboxamide |
| 290. | 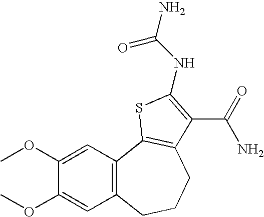 | 2-[(aminocarbonyl)amino]- 8,9-dimethoxy-5,6- dihydro-4H-benzo[6,7]- cyclohepta[1,2-b]- thiophene-3-carboxamide |
| 291. | 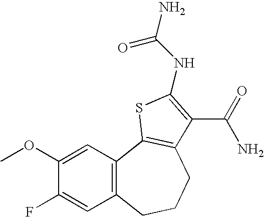 | 2-[(aminocarbonyl)- amino]-8-fluoro-9- methoxy-5,6-dihydro-4H- benzo[6,7]cyclohepta[1,2- b]thiophene-3- carboxamide |
| 292. | 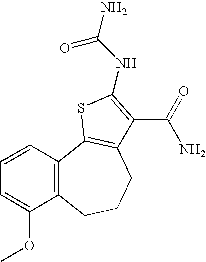 | 2-[(aminocarbonyl)- amino]-7-methoxy-5,6- dihydro-4H-benzo[6,7]- cyclohepta[1,2-b]- thiophene-3-carboxamide |
| 293. | 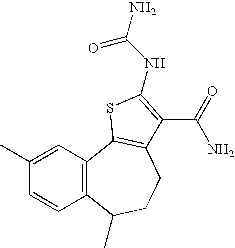 | 2-[(aminocarbonyl)- amino]-6,9-dimethyl-5,6- dihydro-4H-benzo- [6,7lcyclo-hepta[1,2-b]- thiophene-3-carboxamide |
| 294. | 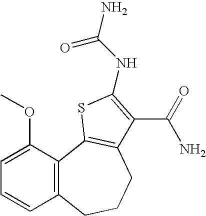 | 2-[(aminocarbonyl)- amino]-10-methoxy-5,6- dihydro-4H-benzo[6,7]- cyclo-hepta[1,2-b]- thiophene-3-carboxamide |
| 295. | 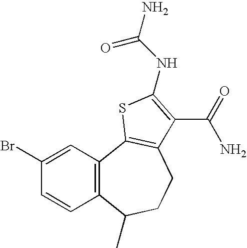 | 2-[(aminocarbonyl)- amino]-9-bromo-6- methyl-5,6-dihydro-4H- benzo[6,7]cyclohepta[1,2- b]thiophene-3- carboxamide |
| 296. | 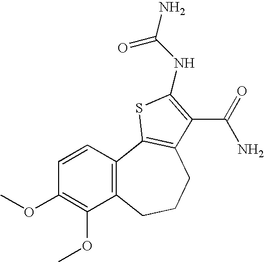 | 2-[(aminocarbonyl)- amino]-7,8-dimethoxy- 5,6-dihydro-4H-benzo- [6,7]cyclohepta[1,2-b]- thiophene-3-carboxamide |
| 297. | 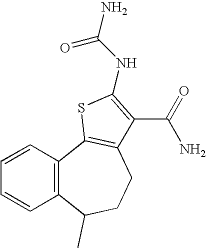 | 2-[(aminocarbonyl)- amino]-6-methyl-5,6- dihydro-4H-benzo- [6,7]-cyclohepta[1,2- b]thiophene-3- carboxamide |
| 298. | 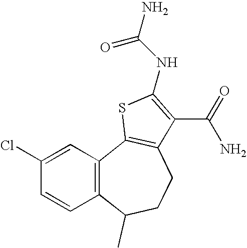 | 2-[(aminocarbonyl)- amino]-9-chloro-6- methyl-5,6-dihydro- 4H-benzo-[6,7]cyclo- hepta[1,2-b]thiophene- 3-carboxamide |
| 299. | 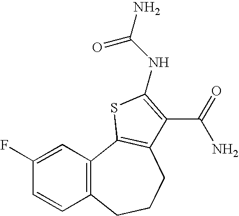 | 2-[(aminocarbonyl)- amino]-9-fluoro-5,6- dihydro-4H-benzo- [6,7]cyclohepta[1,2-b]- thiophene-3- carboxamide |
| 300. | 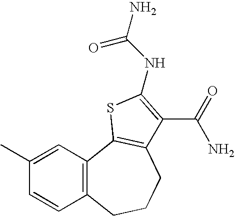 | 2-[(aminocarbonyl)- aminol-9-methyl-5,6- dihydro-4H- benzo[6,7]- cyclohepta[1,2-b]- thiophene-3- carboxamide |
| 301. | 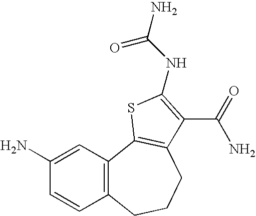 | 9-amino-2-[(amino- carbonyl)aminol-5,6- dihydro-4H-benzo- [6,7]-cyclohepta[1,2- b]thiophene-3- carboxamide |
| 302. | 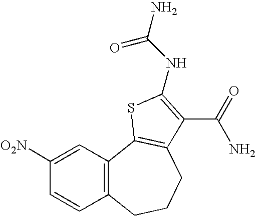 | 2-[(aminocarbonyl)- amino]-9-nitro-5,6- dihydro-4H-benzo- [6,7]cyclohepta[1,2-b]- thiophene-3- carboxamide |
| 303. | 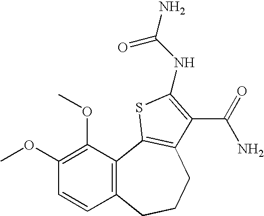 | 2-[(aminocarbonyl)- amino]-9,10- dimethoxy-5,6- dihydro-4H-benzo- [6,7]cyclo-hepta[1,2- b]thiophene-3- carboxamide |

Biological Evaluation

Materials

SAM2™ 96 Biotin capture plates were from Promega. Anti-FLAG affinity resin, FLAG-peptide, NP40 (Nonidet P40), BSA, ATP, ADP, AMP, LPS (E. coli serotype 0111:B4), and dithiothreitol were obtained from Sigma Chemicals. Antibodies specific for NEMO (IKKγ) (FL-419), IKK1(H-744), IKK2(H-470) and IκBα(C-21) were purchased from Santa Cruz Biotechnology. Ni-NTA resin was purchased from Qiagen. Peptides were purchased from American Peptide Company. Protease inhibitor cocktail tablets were from Boehringer Mannheim. Sephacryl S-300 column was from Pharmacia LKB Biotechnology. Centriprep-10 concentrators with a molecular weight cutoff of 10 kDa and membranes with molecular weight cut-off of 30 kDa were obtained from Amicon. [γ-³³P] ATP (2500 Ci/mmol) and [γ-³²P] ATP (6000 Ci/mmol) were purchased from Amersham. The other reagents used were of the highest grade commercially available.

Cloning and Expression

cDNAs of human IKK1 and IKK2 were amplified by reverse transcriptase-polymerase chain reaction from human placental RNA (Clonetech). hIKK1 was subcloned into pFastBac HTa (Life Technologies) and expressed as N-terminal His₆-tagged fusion protein. The hIKK2 cDNA was amplified using a reverse oligonucleotide primer which incorporated the peptide sequence for a FLAG-epitope tag at the C-terminus of the IKK2 coding region (DYKDDDDKD). The hIKK2:FLAG cDNA was subcloned into the baculovirus vector pFastBac. The rhIKK2 (S177S, E177E) mutant was constructed in the same vector used for wild type rhIKK2 using a QuikChange™ mutagenesis kit (Stratagene). Viral stocks of each construct were used to infect insect cells grown in 40L suspension culture. The cells were lysed at a time that maximal expression and rhIKK activity were demonstrated. Cell lysates were stored at −80° C. until purification of the recombinant proteins was undertaken as described below.

Enzyme Isolation

All purification procedures were carried out at 4° C. unless otherwise noted. Buffers used are: buffer A: 20 mM Tris-HCl, pH 7.6, containing 50 mM NaCl, 20 mM NaF, 20 mM β-Glycerophosphate, 500 uM sodiumortho-vanadate, 2.5 mM metabisulfite, 5 mM benzamidine, 1 mM EDTA, 0.5 mM EGTA, 10% glycerol, 1 mM DTT, 1×Complete™ protease inhibitors; buffer B: same as buffer A, except 150 mM NaCl, and buffer C: same as buffer A, except 500 mM NaCl.

Isolation of rhIKK1 homodimer

Cells from an 8-liter fermentation of baculovirus-expressed IKK1 tagged with His peptide were centrifuged and the cell pellet (MOI 0.1, I=72 hr) was re-suspended in 100 ml of buffer C. The cells were microfluidized and centrifuged at 100,000×g for 45 min. The supernatant was collected, imidazole added to the final concentration of 10 mM and incubated with 25 ml of Ni-NTA resin for 2 hrs. The suspension was poured into a 25 ml column and washed with 250 ml of buffer C and then with 125 ml of 50 mM imidazole in buffer C. rhIKK1 homodimer was eluted using 300 mM imidazole in buffer C. BSA and NP-40 were added to the enzyme fractions to the final concentration of 0.1%. The enzyme was dialyzed against buffer B, aliquoted and stored at −80° C.

Isolation of rhIKK2 homodimer

A 10-liter culture of baculovirus-expressing IKK2 tagged with FLAG peptide was centrifuged and the cell pellet (MOI=0.1 and 1=72 hrs) was re-suspended in buffer A. These cells were microfluidized, and centrifuged at 100,000×g for 45 min. Supernatant was passed over a G-25 column equilibrated with Buffer A. Protein peak was collected and incubated with anti-FLAG affinity resin on a rotator overnight in buffer B. The resin was washed in batch with 10-15 bed volumes of buffer C. Washed resin was poured into a column and rhIKK2 homodimer was eluted using 5 bed volumes of buffer B containing FLAG peptide. 5 mM DTT, 0.1% NP-40 and BSA (concentrated to 0.1% in final amount) was added to the eluted enzyme before concentrating in using an Amicon membrane with a molecular weight cut-off of 30 kDa. Enzyme was aliquoted and stored at −80° C.

Isolation of rhIKK1/IKK2 heterodimer

The heterodimer enzyme was produced by coinfection in a baculovirus system (FLAG IKK2/IKK1 His; MOI═0.1 and I=72 hrs). Infected cells were centrifuged and the cell pellet (10.0 g) was suspended in 50 ml of buffer A. The protein suspension was microfluidized and centrifuged at 100,000× g for 45 min. Imidazole was added to the supernatant to a final concentration of 10 mM. The protein was allowed to bind 25 ml of Ni-NTA resin by mixing for 2 hrs. The protein-resin slurry was poured into a 25 ml column and washed with 250 ml of buffer A containing 10 mM imidazole followed by 125 ml of buffer A containing 50 mM imidazole. Buffer A, containing 300 mM imidazole, was then used to elute the protein. A 75 ml pool was collected and NP-40 was added to a final concentration of 0.1%. The protein solution was then dialyzed against buffer B. The dialyzed heterodimer enzyme was then allowed to bind to 25 ml of anti-FLAG M2 agarose affinity gel overnight with constant mixing. The protein-resin slurry was then centrifuged for 5 min at 2,000 rpm. The supernatant was collected and the resin re-suspended in 100 ml of buffer C containing 0.1% NP-40. The resin was washed with 375 ml of buffer C containing 0.1% NP-40. The protein-resin was poured into a 25 ml column and the enzyme eluted using buffer B containing FLAG peptide. Enzyme fractions (100 ml) were collected and concentrated to 20 ml using an Amicon membrane with molecular weight cut-off of 30 kDa. Bovine serum albumin was added to the concentrated enzyme to final concentration of 0.1%. The enzyme was then aliquoted and stored at −80° C.

Cell Culture

The wild type (wt) human pre-B cell line, 70Z/3, and its mutant, 1.3E2, were generously provided by Dr. Carol Sibley. Wt 70Z/3 and 1.3E2 cells were grown in RPMI 1640 (Gibco) supplemented with 7% defined bovine serum (Hyclone) and 50 μM 2-mercaptoethanol. Human monocytic leukemia THP-1 cells, obtained from ATCC, were cultured in RPMI 1640 supplemented with 10% defined bovine serum, 10 mM HEPES, 1.0 mM sodium pyruvate and 50 μM 2-mercaptoethanol. For experiments, cells were plated in 6 well plates at 1×10⁶ cells/ml in fresh media. Pre-B cells were stimulated by the addition of 10 μg/ml LPS for varying lengths of time ranging from 04 hr. THP-1 cells were stimulated by the addition of 1 μg/ml LPS for 45 minutes. Cells were pelleted, washed with cold 50 mM sodium phosphate buffer, pH 7.4 containing 0.15 M NaCl and lysed at 4° C. in 20 mM Hepes buffer, pH 7.6 containing 50 mM NaCl, 1 mM EDTA, 1 mM EGTA, 1 mM sodium orthovanadate, 10 mM β-glycerophosphate, 1 mM NaF, 1 mM PMSF, 1 mM DTT and 0.5% NP40 (lysis buffer). The cytosolic fractions obtained following centrifugation at 10,000×g were stored at −80° C. until used.

Immunoprecipitation and Western Blotting

SF9 cells paste containing rhIKKs were centrifuged (100,000× g, 10 min) to remove debris. rhIKKs were immunoprecipitated (100 μg of cell paste) from the cell supernatant using 3 μg of anti-NEMO antibody (FL-419), followed by coupling to protein A sepharose beads. rhIKKs were also immunoprecipitated from affinity chromatography purified protein preparations (1 μg) using anti-FLAG, anti-His or anti-NEMO antibodies (1-4 μg) followed by protein A sepharose coupling. The native, human IKK complex was immunoprecipitated from THP-1 cell homogenates (300 μg/condition) using the anti-NEMO antibody. Immune complexes were pelleted and washed 3 times with 1 ml cold lysis buffer. Immunoprecipitated rhIKKs were chromatographed by SDS-PAGE (8% Tris-glycine) and transferred to nitrocellulose membranes (Novex) and detected by chemiluminescense (SuperSignal) using specific anti-IKK antibodies (IKK2H-470, IKK1 H-744). Native IKK2, IκBα and NEMO proteins from cytosolic lysates (20-80 μg) were separated by SDS-PAGE and visualized by chemiluminescense using specific antibodies.

Phosphatase Treatment

Immunoprecipitated rhIKKs were washed 2 times in 50 mM Tris-HCl, pH 8.2 containing 0.1 mM EDTA, 1 mM DTT, 1 mM PMSF and 2 mM MnCl₂ and resuspended in 50 l. Phosphatase (λPPase, 1000 U) was pre-diluted in the same buffer and added to the IKK samples. Following incubation at room temperature for 30 minutes with intermittent mixing, cold lysis buffer was added to the tubes to stop the reaction. After several washes, 10% of the beads were removed for Western analysis, and the remaining material was pelleted and resuspended in 100 μl of the buffer used for the in vitro kinase assay.

IKKαSAM Enzyme Assay

IKKα kinase activity was measured using a biotinylated IκBα peptide (Gly-LeuLys-Lys-Glu-Arg-Leu-Leu-Asp-Asp-Arg-His-Asp-Ser₃₂-Gly-Leu-Asp-Ser₃₆-Met Lys-Asp-Glu-Glu), a SAM2™ 96 Biotin capture plate, and a vacuum system. The standard reaction mixture contained 5 μM biotinylated IκBα peptide, 1 μM [γ-³³P] ATP (about 1×10⁵ cpm), 1 mM DTT, 50 mM KCl, 2 mM MgCl₂, 2 mM MnCl₂, 10 mM NaF, 25 mM Hepes buffer, pH. 7.6 and enzyme solution (1-10 μl) in a final volume of 50 l. After incubation at 25° C. for 30 min, 25 μl of the reaction mixture was withdrawn and added to a SAM2™ Biotin capture 96-well plate. Each well was then washed successively with 800 μl 2 M NaCl, 1.2 ml of NaCl containing 1% H₃PO₄, 400 μl H₂O, and 200 μl 95% ethanol. The plate was allowed to dry in a hood at 25° C. for 1 hr and then 25 μl of scintillation fluid (Microscint 20) was added to each well. Incorporation of [γ-³³P] ATP was measured using a Top-Count NXT (Packard). Under each assay condition, the degree of phosphorylation of IκBα peptide substrate was linear with time and concentration for all purified enzymes. Results from the biotinylated peptide assay were confirmed by SDS-PAGE analysis of kinase reaction utilizing a GST-IκBα₁₋₅₄ and [γ-³² P]ATP. The resulting radiolabeled substrate was quantitated by Phosphoimager (Molecular Dynamics). An ion exchange resin assay was also employed using [γ-³³P] ATP and GST-IκBα₁₋₅₄ fusion protein as the substrates. Each assay system yielded consistent results in regard to Kₘ and specific activities for each of the purified kinase isoforms. One unit of enzyme activity was defined as the amount required to catalyze the transfer of 1 nmole of phosphate from ATP to IκBα peptide per min. Specific activity was expressed as units per mg of protein. For experiments related to Kₘ determination of purified enzymes, various concentrations of ATP or IκBα peptide were used in the assay at either a fixed IκBα or ATP concentration. For IκBα peptide Kₘ, assays were carried out with 0.1 μg of enzyme, 5 μM ATP and IκBα peptide from 0.5 to 20 μM. For ATP Kₘ, assays were carried out with 0.1 μg of enzyme, 10 μM IκBα peptide and ATP from 0.1 to 10 μM. For Kₘ determination of rhIKK1 homodimer, due to its low activity and higher Kₘ for IκBα peptide, rhIKK1 homodimer (0.3 μg) was assayed with 125 μM IκBα peptide and a 5-fold higher specific activity of ATP (from 0.1 to 10 μM) for ATP Kₘ experiments and a 5-fold higher specific activity of 5 μM ATP and IκBα peptide (from 5 to 200 μM) for IκBα peptide Kₘ experiments.

IKKβ Resin Enzyme Assay

IKKβ kinase activity was measured using a biotinylated IκBα peptide (Gly-LeuLys-Lys-Glu-Arg-Leu-Leu-Asp-Asp-Arg-His-Asp-Ser₃₂-Gly-Leu-Asp-Ser₃₆-Met Lys-Asp-Glu-Glu) (American Peptide Co.). 20 ul of the standard reaction mixture contained 5 μM biotinylated IκBα peptide, 0.1 μCi/reaction [γ³³P] ATP (Amersham) (about 1×10⁵ cpm), 1 μM ATP (Sigma), 1 mM DTT (Sigma), 2 mM MgCl₂ (Sigma), 2 mM MnCl₂ (Sigma), 10 mM NaF (Sigma), 25 mM Hepes (Sigma) buffer, pH 7.6 and 20 μl enzyme solution and 10 ul inhibitor in a final volume of 50 l. After incubation at 25° C. for 30 min, 150 μl resin (Dowex anion-exchange resin AGIX8 200-400 mesh) in 900 mM formate, pH 3.0 was added to each well to stop the reaction. Resin was allowed to settle for one hour and 50 ul of supernatant was removed to a Micolite-2 flat bottom plate (Dynex). 150 μl of scintillation fluid (Microscint 40) (Packard) was added to each well. Incorporation of [γ-³³P] ATP was measured using a Top-Count NXT (Packard).

IKK Heterodimer Resin Enzyme Assay

IKK heterodimer kinase activity was measured using a biotinylated IκBα peptide (Gly-Leu-Lys-Lys-Glu-Arg-Leu-Leu-Asp-Asp-Arg-His-Asp-Ser₃₂-Gly-Leu-Asp-Ser₃₆-Met-Lys-Asp-Glu-Glu) (American Peptide Co.). 20 ul of the standard reaction mixture contained 5 μM biotinylated IκBα peptide, 0.1 μCi/reaction [γ-³³P] ATP (Amersham) (about 1×10⁵ cpm), 1 μM ATP (Sigma), 1 mM DTT (Sigma), 2 mM MgCl₂ (Sigma), 2 mM MnCl₂ (Sigma), 10 mM NaF (Sigma), 25 mM Hepes (Sigma) buffer, pH 7.6 and 20 μl enzyme solution and 10 μl inhibitor in a final volume of 50 μl. After incubation at 25° C. for 30 min, 150 μl resin (Dowex anion-exchange resin AGIX8 200-400 mesh) in 900 mM formate, pH 3.0 was added to each well to stop the reaction. Resin was allowed to settle for one hour and 50 μl of supernatant was removed to a Micolite-2 flat bottom plate (Dynex). 150 μl of scintillation fluid (Microscint 40) (Packard) was added to each well. Incorporation of [γ-³³P] ATP was measured using a Top-Count NXT (Packard).

What is claimed is:

1 A compound of formula I 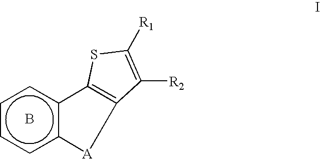
or isomers, tautomers, polymorphs, carriers, prodrugs, pharmaceutically acceptable salts thereof, wherein;
A is (CH₂)ₘ or (CH₂)ₘ—W—(CH₂)ₙ, wherein A is optionally substituted with one or more substituent independently selected from the group consisting of sulfamyl, halo, alkyl, alkoxy, hydroxyl and haloalkyl, CF₃, COCF₃, CN, NO₂, hydrido, OR³, OCOOR³, CO₂H, CO₂R³, CONH₂, CONHR³, CON(R³)₂, COR³, SR³, SOR³, SCOOR³, SO₂R³, NH₂, NHR³, NR³R³, NR³COR³, NR³CONHR³, NR³SO₂R³, NR³SO₂NHR³, SO₂NHR³, and SO₂N(R³)₂;
B is a 6-membered aromatic hydrocarbon ring, optionally substituted with one or more substituent independently selected from the group consisting of OR³, SR⁴, SO₂N(R⁴)₂, NHR⁴, NHCOR⁴, NR⁴COR⁴, NHCO(OR⁴), NR⁴CO(OR⁴), NHCONHR³, NR³CONHR⁴, NR³SO₂R⁴, NHSO₂R⁴, NHSO₂N(R⁴)₂, NHCONR⁴R⁴, CO₂R⁴, CON(R⁴)₂, aryl, heteroaryl, heterocyclic, halo, alkylthio, alkylsulfinyl, alkylsulfonyl, cyano, carboxyl, alkoxycarbonyl, amido, N-monoalkylamido, N-monoarylamido, alkyl, alkenyl, alkynyl, N,N-dialkylamido, N-alkyl-N-arylamido, haloalkyl hydrido, hydroxyl, alkoxy, hydroxyalkyl, haloalkoxy, sulfamyl, N-alkylsulfamyl, amino, alkylamino, and nitro;
W is S(O)ₚ, O, CH═N, N(O)═CH, and NR⁴;
m is 0 to 3, inclusive;
n is 0 to 3, inclusive;
p is 0 to 2, inclusive;
R¹ and R² are independently selected from the group consisting of: hydrido, cyano, nitro, hydroxyl, alkyl, hydroxyalkyl, haloalkyl, alkoxy, haloalkoxy, amidine, guanidine, CONHR⁵, NHR⁵, and NHCXNHR⁵, wherein X is O, S, or NR⁶;
R³ is selected from the group consisting of: hydrido, aryl, heteroaryl, lower alkyl, alkenyl, alkynyl, and heteroalkyl;
R⁴ is selected from the group consisting of: lower alkyl, aryl, heteroaryl, arylalkyl, heteroalkyl, haloalkyl, arylalkylamino, and heteroarylalkyl, wherein aryl, arylalkyl, heteroaryl, or heteroarylalkyl are optionally substituted with one or more radical selected from alkyl, alkoxy, halo, haloalkyl, cyano, haloalkoxy, acyl, carboxyl, hydroxy, hydroxyalkyloxy, phenoxy, benzyloxy, dialkylaminoalkyloxy, and heterocyclic;
R⁵ is selected from the group consisting of: hydrido, alkyl, aminoalkyl, hydroxyalkyl, haloalkyl, and acyl;
R⁶ is selected from the group consisting of: hydrido, alkyl, cyano, and nitro.
With the proviso, when W is CH═N and m is 0, the nitrogen atom in CH═N has to be directly attached to the ring B.

2 The compound of claim 1, wherein A is (CH₂)₂.

3 The compound of claim 1, wherein A is (CH₂)₃.

4 The compound of claim 2 or 3, wherein R¹ is NHR⁵.

5 The compound of claim 4, wherein R¹ is NHCOCH₃.

6 The compound of claim 4 wherein R¹ is NHCONHR⁵.

7 The compound of claim 6 wherein R¹ is NHCONH₂.

8 The compound of claim 2 or 3, wherein R² is CONHR⁵.

9 The compound of claim 8 wherein R² is CONH₂.

10 The compound of claim 8 wherein R¹ is NHCOCH₃ or NHCONH₂.

11 The compound of claim 1 selected from the group consisting of:
3-amino-4,5-dihydronaphtho[1,2-b]thiophene-2-carboxamide,
3-amino-8-methoxy-4,5-dihydronaphtho[1,2-b]thiophene-2-carboxamide,
3-[(2-aminoethyl)amino]-4,5-dihydronaphtho[1,2-b]thiophene-2-carboxamide hydrobromide,
3-[(3-aminopropyl)amino]-4,5-dihydronaphtho[1,2-b]thiophene-2-carboxamide hydrochloride,
3-[(aminocarbonyl)amino]4,5-dihydronaphtho[1,2-b]thiophene-2-carboxamide,
2-amino-4,5-dihydronaphtho[1,2-b]thiophene-3-carbonitrile,
2-(acetylamino)-4,5-dihydronaphtho[1,2-b]thiophene-3-carboxamide, and
2-[(aminocarbonyl)amino]4,5-dihydronaphtho[1,2-b]thiophene-3-carboxamide.

12 The compound of claim 1 selected from the group consisting of:
2-[(aminocarbonyl)amino]-7-methoxy-4,5-dihydronaphtho[1,2-b]-thiophene-3-carboxamide,
2-[(aminocarbonyl)amino]-6-methoxy-4,5-dihydronaphtho[1,2-b]-thiophene-3-carboxamide,
2-[(aminocarbonyl)amino]-6,7-dimethoxy-4,5-dihydronaphtho[1,2-b]-thiophene-3-carboxamide,
2-[(aminocarbonyl)amino]-7-isopropyl-8-methoxy-4,5-dihydronaphtho[
1,2-b]thiophene-3-carboxamide,
2-[(aminocarbonyl)amino]-4,4-dimethyl-4,5-dihydronaphtho[1,2-b]-thiophene-3-carboxamide,
2-[(aminocarbonyl)amino]-4-methyl-4,5-dihydronaphtho[1,2-b]thiophene-3-carboxamide,
2-[(aminocarbonyl)amino]-7-fluoro-4,5-dihydronaphtho[1,2-b]thiophene-3-carboxamide,
2-[(aminocarbonyl)amino]-8-fluoro-4,5-dihydronaphtho[1,2-b]thiophene-3-carboxamide,
2-[(aminocarbonyl)amino]-4-ethyl-4-methyl-4,5-dihydronaphtho[1,2-b]thiophene-3-carboxamide,
2-[(aminocarbonyl)amino]-4,4-diethyl-4,5-dihydronaphtho[1,2-b]-thiophene-3-carboxamide,
2-[(aminocarbonyl)amino]-8-methoxy-4,5-dihydronaphtho[1,2-b]-thiophene-3-carboxamide,
2-[(aminocarbonyl)amino]-9-methoxy-4,5-dihydronaphtho[1,2-b]thiophene-3-carboxamide,
2-[(aminocarbonyl)amino]-7-methoxy-5,5-dimethyl-4,5-dihydronaphtho[
1,2-b]thiophene-3-carboxamide,
2-[(aminocarbonyl)amino]-9-ethoxy-4,5-dihydronaphtho[1,2-b]-thiophene-3-carboxamide,
2-[(aminocarbonyl)amino]-7,8-dimethoxy-4,5-dihydronaphtho[1,2-b]-thiophene-3-carboxamide,
2-[(aminocarbonyl)amino]-6,9-dimethoxy-4,5-dihydronaphtho[1,2-b]-thiophene-3-carboxamide,
2-[(aminocarbonyl)amino]-6-methyl-4,5-dihydronaphtho[1,2-b]thiophene-3-carboxamide,
2-[(aminocarbonyl)amino]-9-methyl-4,5-dihydronaphtho[1,2-b]thiophene-3-carboxamide,
2-[(aminocarbonyl)amino]-7-methyl-4,5-dihydronaphtho[1,2-b]-thiophene-3-carboxamide,
2-[(aminocarbonyl)amino]-8-methyl-4,5-dihydronaphtho[1,2-b]thiophene-3-carboxamide,
2-[(aminocarbonyl)amino]-7,9-dimethoxy-4,5-dihydronaphtho[1,2-b]-thiophene-3-carboxamide,
2-[(aminocarbonyl)amino]-6,7-dimethyl-4,5-dihydronaphtho-[1,2-b]-thiophene-3-carboxamide,
2-[(aminocarbonyl)amino]-7-ethyl-4,5-dihydronaphtho[1,2-b]thiophene-3-carboxamide,
2-[(aminocarbonyl)amino]-7,8-dimethyl-4,5-dihydronaphtho-[1,2-b]-thiophene-3-carboxamide,
2-[(aminocarbonyl)amino]-7-tert-butyl-4,5-dihydronaphtho-[1,2-b]-thiophene-3-carboxamide,
2-[(aminocarbonyl)amino]-7-propyl-4,5-dihydronaphtho[1,2-b]thiophene-3-carboxamide,
2-[(aminocarbonyl)amino]-6-ethoxy-4,5-dihydronaphtho[1,2-b]thiophene-3-carboxamide,
2-[(affinocarbonyl)amino]-7-butyl-4,5-dihydronaphtho[1,2-b]thiophene-3-carboxamide,
2-[(aminocarbonyl)amino]-9-propoxy-4,5-dihydronaphtho[1,2-b]thiophene-3-carboxamide,
2-[(aminocarbonyl)amino]-9-isopropoxy-4,5-dihydronaphtho-[1,2-b]thiophene-3-carboxamide,
2-[(aminocarbonyl)amino]-9-butoxy-4,5-dihydronaphtho[1,2-b]thiophene-3-carboxamide,
2-[(aminocarbonyl)amino]-9-isobutoxy-4,5-dihydronaphtho[1,2-b]thiophene-3-carboxamide,
2-[(aminocarbonyl)amino]-9-sec-butoxy-4,5-dihydronaphtho-[1,2-b]thiophene-3-carboxamide,
2-[(aminocarbonyl)amino]-7-methoxy-6-methyl-4,5-dihydronaphtho[1,2-b]thiophene-3-carboxamide,
2-[(aminocarbonyl)amino]-8-methoxy-9-methyl-4,5-dihydronaphtho[1,2-b]thiophene-3-carboxamide,
2-[(aminocarbonyl)amino]-6-isopropyl-8-methoxy-9-methyl-4,5-dihydronaphtho[
1,2-b]thiophene-3-carboxamide,
2-[(aminocarbonyl)amino]-7-isopropyl-6-methoxy-4,5-dihydronaphtho[1,2-b]thiophene-3-carboxamide,
2-[(aminocarbonyl)amino]-7-isopropyl-4,5-dihydronaphtho-[1,2-b]thiophene-3-carboxamide,
2-[(aminocarbonyl)amino]-7-bromo-4,5-dihydronaphtho[1,2-b]thiophene-3-carboxamide,
2-[(aminocarbonyl)amino]-5,5,8-trimethyl-4,5-dihydronaphtho-[1,2-b]thiophene-3-carboxamide,
2-[(aminocarbonyl)amino]-6-chloro-4,5-dihydronaphtho[1,2-b]thiophene-3-carboxamide,
2-[(aminocarbonyl)amino]-7-chloro-4,5-dihydronaphtho[1,2-b]thiophene-3-carboxamide,
2-[(aminocarbonyl)amino]-8-chloro-4,5-dihydronaphtho[1,2-b]thiophene-3-carboxamide,
2-[(aminocarbonyl)amino]-6,8-dichloro-4,5-dihydronaphtho[1,2-b]thiophene-3-carboxamide,
2-[(aminocarbonyl)amino]-7,8-dichloro-4,5-dihydronaphtho[1,2-b]thiophene-3-carboxamide,
2-[(aminocarbonyl)amino]-5,5-dimethyl-4,5-dihydronaphtho[1,2-b]thiophene-3-carboxamide,
2-[(aminocarbonyl)amino]-4,5,5-trimethyl-4,5-dihydronaphtho[1,2-b]thiophene-3-carboxamide,
2-[(aminocarbonyl)amino]-8-hexyl-4,5-dihydronaphtho[1,2-b]thiophene-3-carboxamide,
6-(acetylamino)-2-[(aminocarbonyl)amino]4,5-dihydronaphtho[1,2-b]thiophene-3-carboxamide,
2-[(aminocarbonyl)amino]-9-bromo-4,5-dihydronaphtho[1,2-b]thiophene-3-carboxamide,
2-[(aminocarbonyl)amino]-6-fluoro-9-methoxy-4,5-dihydronaphtho[1,2-b]thiophene-3-carboxamide,
2-[(aminocarbonyl)amino]4-ethyl-4,5-dihydronaphtho[1,2-b]thiophene-3-carboxamide,
2-[(aminocarbonyl)amino]-6,9-dimethyl-4,5-dihydronaphtho[1,2-b]thiophene-3-carboxamide,
2-[(aminocarbonyl)-mino]-7-ethoxy-4,5-dihydronaphtho[1,2-b]thiophene-3-carboxamide,
2-[(aminocarbonyl)amino]-7-propoxy-4,5-dihydronaphtho[1,2-b]thiophene-3-carboxamide,
2-[(aminocarbonyl)amino]-8-ethoxy-4,5-dihydronaphtho[1,2-b]thiophene-3-carboxamide,
2-[(aminocarbonyl)amino]-5-methyl-4,5-dihydronaphtho[1,2-b]thiophene-3-carboxamide,
2-[(aminocarbonyl)amino]-7-hydroxy-8-methoxy-4,5-dihydronaphtho[1,2-b]thiophene-3-carboxamide,
2-[(aminocarbonyl)amino]-8-nitro-4,5-dihydronaphtho[1,2-b]thiophene-3-carboxamide,
2-[(aminocarbonyl)amino]-6,8-dimethoxy-4,5-dihydronaphtho[1,2-b]thiophene-3-carboxamide,
2-[(aminocarbonyl)amino]-6-fluoro-4,5-dihydronaphtho[1,2-b]thiophene-3-carboxamide,
2-[(aminocarbonyl)amino]-9-fluoro-4,5-dihydronaphtho[1,2-b]thiophene-3-carboxamide,
2-[(aminocarbonyl)amino]-6-methoxy-4-methyl-4,5-dihydronaphtho[1,2-b]thiophene-3-carboxamide,
2-[(aminocarbonyl)amino]-8-methoxy-4-methyl-4,5-dihydronaphtho[1,2-b]thiophene-3-carboxamide,
2-[(aminocarbonyl)amino]-6-chloro-7-methoxy-4,5-dihydronaphtho[1,2-b]thiophene-3-carboxamide,
2-[(aminocarbonyl)amino]-8-(hydroxy-methyl)-7-methoxy-4,5-dihydronaphtho[
1,2-b]thiophene-3-carboxamide,
2-[(aminocarbonyl)amino]-6-bromo-4,5-dihydronaphtho[1,2-b]thiophene-3-carboxamide,
2-[(aminocarbonyl)amino]-6-iodo-4,5-dihydronaphtho[1,2-b]thiophene-3-carboxamide,
2-[(aminocarbonyl)amino]-8-bromo-4,5-dihydronaphtho[1,2-b]thiophene-3-carboxamide,
2-[(aminocarbonyl)amino]-8-iodo-4,5-dihydronaphtho[1,2-b]thiophene-3-carboxamide,
2-[(aminocarbonyl)amino]-6,8-dimethyl-4,5-dihydronaphtho-[1,2-b]thiophene-3-carboxamide,
2-[(aminocarbonyl)amino]-6-(methylthio)-4,5-dihydronaphtho-[1,2-b]thiophene-3-carboxamide,
2-[(aminocarbonyl)amino]-6-(methyl-sulfonyl)-4,5-dihydronaphtho[1,2-b]thiophene-3-carboxamide,
2-[(aminocarbonyl)amino]-8-ethynyl-4,5-dihydronaphtho[1,2-b]thiophene-3-carboxamide,
2-[(aminocarbonyl)amino]-8-chloro-5-methyl-4,5-dihydronaphtho[1,2-b]thiophene-3-carboxamide,
2-[(aminocarbonyl)amino]-8-(methylthio)-4,5-dihydronaphtho[1,2-b]thiophene-3-carboxamide,
2-[(aminocarbonyl)amino]-8-(methyl-sulfonyl)-4,5-dihydronaphtho[1,2-b]thiophene-3-carboxamide,
2-[(aminocarbonyl)amino]-7-chloro-5-methyl-4,5-dihydronaphtho[1,2-b]thiophene-3-carboxamide,
2-[(aminocarbonyl)amino]-8-(ethylthio)-4,5-dihydronaphtho[1,2-b]thiophene-3-carboxamide,
2-[(aminocarbonyl)amino]-8-(ethyl-sulfonyl)-4,5-dihydronaphtho[1,2-b]thiophene-3-carboxamide,
2-[(aminocarbonyl)amino]-6-chloro-5-methyl-4,5-dihydronaphtho[1,2-b]thiophene-3-carboxamide,
2-[(aminocarbonyl)amino]-6-(ethylthio)-4,5-dihydronaphtho[1,2-b]thiophene-3-carboxamide,
2-[(aminocarbonyl)amino]-6-(ethyl-sulfonyl)-4,5-dihydronaphtho[1,2-b]thiophene-3-carboxamide,
2-[(aminocarbonyl)amino]-9-chloro-4,5-dihydronaphtho[1,2-b]thiophene-3-carboxamide,
2-[(aminocarbonyl)amino]-9-methoxy-4,4-dimethyl-4,5-dihydronaphtho[1,2-b-]thiophene-3-carboxamide,
2-[(aminocarbonyl)amino]-8-methoxy-7-methyl-4,5-dihydronaphtho[1,2-b]thiophene-3-carboxamide,
2-[(aminocarbonyl)amino]-7-methoxy-8-methyl-4,5-dihydronaphtho[1,2-b]thiophene-3-carboxamide,
2-[(aminocarbonyl)-amino]-6-methoxy-7-methyl-4,5-dihydronaphtho[1,2-b]thiophene-3-carboxamide,
2-[(aminocarbonyl)-amino]-9-methoxy-4-methyl-4,5-dihydronaphtho[1,2-b]thiophene-3-carboxamide,
2-[(aminocarbonyl)-amino]-6,7-diethoxy-4,5-dihydronaphtho[-1,2-b]thiophene-3-carboxamide,
2-[(aminocarbonyl)-amino]-8-ethyl-4,5-dihydronaphtho[1,2-b]thiophene-3-carboxamide,
2-[(aminocarbonyl)-amino]-8-isopropyl-4,5-dihydronaphtho[1,2-b]thiophene-3-carboxamide,
2-[(aminocarbonyl)-amino]-7,9-dimethyl-4,5-dihydronaphtho[1,2-b]thiophene-3-carboxamide,
2-[(aminocarbonyl)-amino]-8-tert-butyl-4,5-dihydronaphtho[1,2-b]thiophene-3-carboxamide,
2-[(aminocarbonyl)-amino]-7-methoxy-9-methyl-4,5-dihydronaphtho[1,2-b]thiophene-3-carboxamide,
2-[(aminocarbonyl)-amino]-6,8-difluoro-4,5-dihydronaphtho[1,2-b]thiophene-3-carboxamide,
2-[(aminocarbonyl)-amino]-7-isopropoxy-4,5-dihydronaphtho[1,2-b]thiophene-3-carboxamide,
2-[(aminocarbonyl)-amino]-6-chloro-9-methoxy-4,5-dihydronaphtho[1,2-b]thiophene-3-carboxamide,
2-[(aminocarbonyl)amino]-7,9-dichloro-4,5-dihydronaphtho[1,2-b]thiophene-3-carboxamide,
2-[(aminocarbonyl)amino]-8-(trifluoro-methyl)-4,5-dihydronaphtho[1,2-b]thiophene-3-carboxamide,
2-[(aminocarbonyl)amino]-9-methoxy-6-(methylthio)-4,5-dihydronaphtho[1,2-b]thiophene-3-carboxamide,
2-[(aminocarbonyl)amino]-9-methoxy-6-(methylsulfonyl)-4,5-dihydronaphtho[
1,2-b]thiophene-3-carboxamide,
2-[(aminocarbonyl)amino]-6-ethoxy-9-methoxy-4,5-dihydronaphtho[1,2-b]thiophene-3-carboxamide,
2-[(aminocarbonyl)amino]-6,9-diethoxy-4,5-dihydronaphtho[1,2-b]thiophene-3-carboxamide,
2-[(aminocarbonyl)amino]-8,9-dimethyl-4,5-dihydronaphtho-[1,2-b]thiophene-3-carboxamide,
2-[(aminocarbonyl)amino]-7,8-dinitro-4,5-dihydronaphtho[1,2-b]thiophene-3-carboxamide,
2-[(aminocarbonyl)amino]-7-nitro-4,5-dihydronaphtho[1,2-b]thiophene-3-carboxamide,
2-[(aminocarbonyl)amino]-8-methoxy-4,7-dimethyl-4,5-dihydronaphtho[1,2-b]thiophene-3-carboxamide,
2-[(aminocarbonyl)amino]-6-methoxy-5-methyl-4,5-dihydronaphtho[1,2-b]thiophene-3-carboxamide,
2-[(aminocarbonyl)amino]-7-iodo-4,5-dihydronaphtho[1,2-b]thiophene-3-carboxamide,
2-[(aminocarbonyl)amino]-7-chloro-8-nitro-4,5-dihydronaphtho[1,2-b]thiophene-3-carboxamide,
2-[(aminocarbonyl)amino]-5,8-dimethyl-4,5-dihydronaphtho-[1,2-b]thiophene-3-carboxamide,
2-[(aminocarbonyl)amino]-5-ethyl-8-methyl-4,5-dihydronaphtho[1,2-b]thiophene-3-carboxamide,
benzyl 3-(aminocarbonyl)-2-[(amino-carbonyl)amino]4,5-dihydronaphtho[1,2-b]thien-8-ylcarbamate,
8-amino-2-[(aminocarbonyl)amino]4,5-dihydronaphtho[1,2-b]thiophene-3-carboxamide,
8-(acetylamino)-2-[(aminocarbonyl)amino]-4,5-dihydronaphtho[1,2-b]thiophene-3-carboxamide,
2-[(aminocarbonyl)amino]-5-isopropyl-4,5-dihydronaphtho[1,2-b]thiophene-3-carboxamide,
2-[(aminocarbonyl)amino]-6,9-dichloro-4,5-dihydronaphtho[1,2-b]thiophene-3-carboxamide,
2-[(aminocarbonyl)amino]-9-tert-butyl-4,5-dihydronaphtho[1,2-b]thiophene-3-carboxamide,
2-[(aminocarbonyl)amino]-5,5,7-trimethyl-4,5-dihydronaphtho[1,2-b]thiophene-3-carboxamide,
2-[(aminocarbonyl)amino]-5-butyl-4,5-dihydronaphtho[1,2-b]thiophene-3-carboxamide,
2-[(aminocarbonyl)amino]-5-butyl-7-methyl-4,5-dihydronaphtho[1,2-b]thiophene-3-carboxamide,
2-[(aminocarbonyl)amino]-5-butyl-7-fluoro-4,5-dihydronaphtho[1,2-b]thiophene-3-carboxamide,
2-[(aminocarbonyl)amino]-5-butyl-7-methoxy-4,5-dihydronaphtho[1,2-b]thiophene-3-carboxamide,
2-[(aminocarbonyl)amino]-5-butyl-7,8-dimethoxy-4,5-dihydronaphtho[1,2-b]-thiophene-3-carboxamide,
2-(acetylamino)-4,5-dihydronaphtho[1,2-b]thiophene-3-carboxamide,
2-(acetylamino)-7-methoxy-4,5-dihydronaphtho[1,2-b]thiophene-3-carboxamide,
2-(acetylamino)-6-methoxy-4,5-dihydronaphtho[1,2-b]thiophene-3-carboxamide,
2-(acetylamino)-6,7-dimethoxy-4,5-dihydronaphtho[1,2-b]thiophene-3-carboxamide,
2-(acetylamino)-7-isopropyl-8-methoxy-4,5-dihydronaphtho-[1,2-b]thiophene-3-carboxamide,
2-(acetylamino)4,4-dimethyl-4,5-dihydronaphtho[1,2-b]thiophene-3-carboxamide,
2-(acetylamino)4-methyl-4,5-dihydronaphtho[1,2-b]thiophene-3-carboxamide,
2-(acetylamino)-7-fluoro-4,5-dihydronaphtho[1,2-b]thiophene-3-carboxamide,
2-(acetylamino)-8-fluoro-4,5-dihydronaphtho[1,2-b]thiophene-3-carboxamide,
2-(acetylamino)-4-ethyl-4-methyl-4,5-dihydronaphtho[1,2-b]thiophene-3-carboxamide,
2-(acetylamino)-4,4-diethyl-4,5-dihydronaphtho[1,2-b]thiophene-3-carboxamide,
2-(acetylamino)-8-methoxy-4,5-dihydronaphtho[1,2-b]thiophene-3-carboxamide,
2-(acetylamino)-9-methoxy-4,5-dihydronaphtho[1,2-b]thiophene-3-carboxamide,
2-(acetylamino)-7-methoxy-5,5-dimethyl-4,5-dihydronaphtho-[1,2-b]thiophene-3-carboxamide,
2-(acetylamino)-9-ethoxy-4,5-dihydronaphtho[1,2-b-thiophene-3-carboxamide,
2-(acetylamino)-7,8-dimethoxy-4,5-dihydronaphtho[1,2-b]thiophene-3-carboxamide,
2-(acetylamino)-6,9-dimethoxy-4,5-dihydronaphtho[1,2-b]thiophene-3-carboxamide,
2-(acetylamino)-6-methyl-4,5-dihydronaphtho[1,2-b]thiophene-3-carboxamide,
2-(acetylamino)-9-methyl-4,5-dihydronaphtho[1,2-b]thiophene-3-carboxamide,
2-(acetylamino)-7-methyl-4,5-dihydronaphtho[1,2-b]thiophene-3-carboxamide,
2-(acetylamino)-8-methyl-4,5-dihydronaphtho[1,2-b]thiophene-3-carboxamide
2-(acetylamino)-7,9-dimethoxy-4,5-dihydronaphtho[1,2-b]thiophene-3-carboxamide,
2-(acetylamino)-6,7-dimethyl-4,5-dihydronaphtho[1,2-b]thiophene-3-carboxamide,
2-(acetylamino)-7-ethyl-4,5-dihydronaphtho[1,2-b]thiophene-3-carboxamide,
2-(acetylamino)-7,8-dimethyl-4,5-dihydronaphtho[1,2-b]thiophene-3-carboxamide,
2-(acetylamino)-7-tert-butyl-4,5-dihydronaphtho[1,2-b]thiophene-3-carboxamide,
2-(acetylamino)-7-propyl-4,5-dihydronaphtho[1,2-b]thiophene-3-carboxamide,
2-(acetylamino)-6-ethoxy-4,5-dihydronaphtho[1,2-b]thiophene-3-carboxamide,
2-(acetylamino)-7-butyl-4,5-dihydronaphtho[1,2-b]thiophene-3-carboxamide,
2-(acetylamino)-9-propoxy-4,5-dihydronaphtho[1,2-b]thiophene-3-carboxamide,
2-(acetylamino)-9-isopropoxy-4,5-dihydronaphtho[1,2-b]thiophene-3-carboxamide,
2-(acetylamino)-9-butoxy-4,5-dihydronaphtho[1,2-b]thiophene-3-carboxamide,
2-(acetylamino)-9-isobutoxy-4,5-dihydronaphtho[1,2-b]thiophene-3-carboxamide,
2-(acetylamino)-9-sec-butoxy-4,5-dihydronaphtho[1,2-b]thiophene-3-carboxamide,
2-(acetylamino)-7-methoxy-6-methyl-4,5-dihydronaphtho[1,2-b]thiophene-3-carboxamide,
2-(acetylamino)-8-methoxy-9-methyl-4,5-dihydronaphtho[1,2-b]thiophene-3-carboxamide,
2-(acetylamino)-6-isopropyl-8-methoxy-9-methyl-4,5-dihydronaphtho[
1,2-b]thiophene-3-carboxamide,
2-(acetylamino)-7-isopropyl-6-methoxy-4,5-dihydronaphtho[1,2-b]thiophene-3-carboxamide,
2-(acetylamino)-7-isopropyl-4,5-dihydronaphtho[1,2-b]thiophene-3-carboxamide,
2-(acetylamino)-7-bromo-4,5-dihydronaphtho[1,2-b]thiophene-3-carboxamide,
2-(acetylamino)-5,5,8-trimethyl-4,5-dihydronaphtho[1,2-b]thiophene-3-carboxamide,
2-(acetylamino)-6-chloro-4,5-dihydronaphtho[1,2-b]thiophene-3-carboxamide,
2-(acetylamino)-7-chloro-4,5-dihydronaphtho[1,2-b]thiophene-3-carboxamide,
2-(acetylamino)-8-chloro-4,5-dihydronaphtho[1,2-b]thiophene-3-carboxamide,
2-(acetylamino)-6,8-dichloro-4,5-dihydronaphtho[1,2-b]thiophene-3-carboxamide,
2-(acetylamino)-7,8-dichloro-4,5-dihydronaphtho[1,2-b]thiophene-3-carboxamide,
2-(acetylamino)-5,5-dimethyl-4,5-dihydronaphtho[1,2-b]thiophene-3-carboxamide,
2-(acetylamino)-4,5,5-trimethyl-4,5-dihydronaphtho[1,2-b]thiophene-3-carboxamide,
2-(acetylamino)-8-hexyl-4,5-dihydronaphtho[1,2-b]thiophene-3-carboxamide,
2,6-bis(acetylamino)-4,5-dihydronaphtho-[1,2-b]thiophene-3-carboxamide,
2-(acetylamino)-9-bromo-4,5-dihydronaphtho[1,2-b]thiophene-3-carboxamide,
2-(acetylamino)-6-fluoro-9-methoxy-4,5-dihydronaphtho[1,2-b]thiophene-3-carboxamide,
2-(acetylamino)-4-ethyl-4,5-dihydronaphtho[1,2-b]thiophene-3-carboxamide,
2-(acetylamino)-6,9-dimethyl-4,5-dihydronaphtho[1,2-b]thiophene-3-carboxamide,
2-(acetylamino)-7-ethoxy-4,5-dihydronaphtho[1,2-b]thiophene-3-carboxamide,
2-(acetylamino)-7-propoxy-4,5-dihydronaphtho[1,2-b]thiophene-3-carboxamide,
2-(acetylamino)-8-ethoxy-4,5-dihydronaphtho[1,2-b]thiophene-3-carboxamide,
2-(acetylamino)-5-methyl-4,5-dihydronaphtho[1,2-b]thiophene-3-carboxamide,
2-(acetylamino)-7-hydroxy-8-methoxy-4,5-dihydronaphtho[1,2-b]thiophene-3-carboxamide,
2-(acetylamino)-8-nitro-4,5-dihydronaphtho[1,2-b]thiophene-3-carboxamide,
2-(acetylamino)-6,8-dimethoxy-4,5-dihydronaphtho[1,2-b]thiophene-3-carboxamide,
2-(acetylamino)-6-fluoro-4,5-dihydronaphtho[1,2-b]thiophene-3-carboxamide,
2-(acetylamino)-9-fluoro-4,5-dihydronaphtho[1,2-b]thiophene-3-carboxamide,
2-(acetylamino)-6-methoxy-4-methyl-4,5-dihydronaphtho[1,2-b]thiophene-3-carboxamide
2-(acetylamino)-8-methoxy-4-methyl-4,5-dihydronaphtho[1,2-b]thiophene-3-carboxamide,
2-(acetylamino)-6-chloro-7-methoxy-4,5-dihydronaphtho[1,2-b]thiophene-3-carboxamide,
2-(acetylamino)-8-(hydroxymethyl)-7-methoxy-4,5-dihydronaphtho[1,2-b]thiophene-3-carboxamide,
2-(acetylamino)-6-bromo-4,5-dihydronaphtho[1,2-b]thiophene-3-carboxamide,
2-(acetylamino)-6-iodo-4,5-dihydronaphtho[1,2-b]thiophene-3-carboxamide,
2-(acetylamino)-8-bromo-4,5-dihydronaphtho[1,2-b]thiophene-3-carboxamide,
2-(acetylamino)-8-iodo-4,5-dihydronaphtho[1,2-b]thiophene-3-carboxamide,
2-(acetylamino)-6,8-dimethyl-4,5-dihydronaphtho[1,2-b]thiophene-3-carboxamide,
2-(acetylamino)-6-(methylthio)4,5-dihydronaphtho[1,2-b]thiophene-3-carboxamide,
2-(acetylamino)-6-(methylsulfonyl)-4,5-dihydronaphtho[1,2-b]thiophene-3-carboxamide,
2-(acetylamino)-8-ethynyl-4,5-dihydronaphtho[1,2-b]thiophene-3-carboxamide,
2-(acetylamino)-8-chloro-5-methyl-4,5-dihydronaphtho[1,2-b]thiophene-3-carboxamide,
2-(acetylamino)-8-(methylthio)-4,5-dihydronaphtho[1,2-b]thiophene-3-carboxamide,
2-(acetylamino)-8-(methylsulfonyl)-4,5-dihydronaphtho[1,2-b]thiophene-3-carboxamide,
2-(acetylamino)-8-(ethylthio)-4,5-dihydronaphtho[1,2-b]thiophene-3-carboxamide,
2-(acetylamino)-8-(ethylsulfonyl)-4,5-dihydronaphtho[1,2-b]thiophene-3-carboxamide,
2-(acetylamino)-6-chloro-5-methyl-4,5-dihydronaphtho[1,2-b]thiophene-3-carboxamide,
2-(acetylamino)-6-(ethylthio)-4,5-dihydronaphtho[1,2-b]thiophene-3-carboxamide,
2-(acetylamino)-6-(ethylsulfonyl)-4,5-dihydronaphtho[1,2-b]thiophene-3-carboxamide,
2-(acetylamino)-9-chloro-4,5-dihydronaphtho[1,2-b]thiophene-3-carboxamide,
2-(acetylamino)-9-methoxy-4,4-dimethyl-4,5-dihydronaphtho[1,2-b]thiophene-3-carboxamide,
2-(acetylamino)-8-methoxy-7-methyl-4,5-dihydronaphtho[1,2-b]thiophene-3-carboxamide,
2-(acetylamino)-7-methoxy-8-methyl-4,5-dihydronaphtho[1,2-b]thiophene-3-carboxamide,
2-(acetylamino)-6-methoxy-7-methyl-4,5-dihydronaphtho[1,2-b]thiophene-3-carboxamide,
2-(acetylamino)-9-methoxy-4-methyl-4,5-dihydronaphtho[1,2-b]thiophene-3-carboxamide,
2-(acetylamino)-6,7-diethoxy-4,5-dihydronaphtho[1,2-b]thiophene-3-carboxamide,
2-(acetylamino)-8-ethyl-4,5-dihydro-naphtho[1,2-b]-thiophene-3-carboxamide,
2-(acetylamino)-8-isopropyl-4,5-dihydronaphtho[1,2-b]thiophene-3-carboxamide,
2-(acetylamino)-7,9-dimethyl-4,5-dihydronaphtho[1,2-b]thiophene-3-carboxamide
2-(acetylamino)-8-tert-butyl-4,5-dihydronaphtho[1,2-b]thiophene-3-carboxamide,
2-(acetylamino)-7-methoxy-9-methyl-4,5-dihydronaphtho[1,2-b]thiophene-3-carboxamide,
2-(acetylamino)-6,8-difluoro-4,5-dihydronaphtho[1,2-b]thiophene-3-carboxamide,
2-(acetylamino)-7-isopropoxy-4,5-dihydronaphtho[1,2-b]thiophene-3-carboxamide,
2-(acetylamino)-6-chloro-9-methoxy-4,5-dihydronaphtho[1,2-b]thiophene-3-carboxamide,
2-(acetylamino)-7,9-dichloro-4,5-dihydronaphtho[1,2-b-thiophene-3-carboxamide,
2-(acetylamino)-8-(trifluoromethyl)-4,5-dihydronaphtho[1,2-b]thiophene-3-carboxamide,
2-(acetylamino)-9-methoxy-6-(methylthio)-4,5-dihydronaphtho[1,2-b]thiophene-3-carboxamide,
2-(acetylamino)-9-methoxy-6-(methyl-sulfonyl)-4,5-dihydronaphtho[1,2-b]thiophene-3-carboxamide,
2-(acetylamino)-6-ethoxy-9-methoxy-4,5-dihydronaphtho-[1,2-b]thiophene-3-carboxamide,
2-(acetylamino)-6,9-diethoxy-4,5-dihydronaphtho[1,2-b]thiophene-3-carboxamide,
2-(acetylamino)-8,9-dimethyl-4,5-dihydronaphtho[1,2-b]thiophene-3-carboxamide,
2-(acetylamino)-7,8-dinitro-4,5-dihydronaphtho[1,2-b]thiophene-3-carboxamide,
2-(acetylamino)-7-nitro-4,5-dihydronaphtho[1,2-b]thiophene-3-carboxamide,
2-(acetylamino)-8-methoxy-4,7-dimethyl-4,5-dihydronaphtho[1,2-b]thiophene-3-carboxamide,
2-(acetylamino)-6-methoxy-5-methyl-4,5-dihydronaphtho[1,2-b]thiophene-3-carboxamide,
2-(acetylamino)-7-iodo-4,5-dihydronaphtho[1,2-b]thiophene-3-carboxamide,
2-(acetylamino)-7-chloro-8-nitro-4,5-dihydronaphtho[1,2-b]thiophene-3-carboxamide,
2-(acetylamino)-5,8-dimethyl-4,5-dihydronaphtho[1,2-b]thiophene-3-carboxamide,
2-(acetylamino)-5-ethyl-8-methyl-4,5-dihydronaphtho[1,2-b]thiophene-3-carboxamide,
benzyl 2-(acetyl-amino)-3-(amino-carbonyl)-4,5-dihydronaphtho[1,2-b]thien-8-ylcarbamate,
2-(acetylamino)-8-amino-4,5-dihydronaphtho[1,2-b]thiophene-3-carboxamide,
2,8-bis(acetylamino)-4,5-dihydronaphtho-[1,2-b]thiophene-3-carboxamide,
2-(acetylamino)-5-isopropyl-4,5-dihydronaphtho[1,2-b]thiophene-3-carboxamide,
2-(acetylamino)-6,9-dichloro-4,5-dihydronaphtho[1,2-b]thiophene-3-carboxamide,
2-(acetylamino)-9-tert-butyl-4,5-dihydronaphtho[1,2-b]thiophene-3-carboxamide,
2-(acetylamino)-5,5,7-trimethyl-4,5-dihydronaphtho[1,2-b]thiophene-3-carboxamide,
2-(acetyl amino)-5-butyl-4,5-dihydronaphtho[1,2-b]thiophene-3-carboxamide,
2-(acetylamino)-5-butyl-7-methyl-4,5-dihydronaphtho[1,2-b]thiophene-3-carboxamide,
2-(acetylamino)-5-butyl-7-fluoro-4,5-dihydronaphtho[1,2-b]thiophene-3-carboxamide,
2-(acetylamino)-5-butyl-7-methoxy-4,5-dihydronaphtho[1,2-b]thiophene-3-carboxamide,
2-(acetylamino)-5-butyl-7,8-dimethoxy-4,5-dihydronaphtho-[1,2-b]thiophene-3-carboxamide,
2-(acetylamino)-5,6-dihydro-4H-benzo-[6,7]-cyclo-hepta[1,2-b]thiophene-3-carboxamide,
2-(acetylamino)-9-chloro-5,6-dihydro-4H-benzo[6,7]-cyclo-hepta[1,2-b]thiophene-3-carboxamide,
2-(acetylamino)-8,9-dichloro-5,6-dihydro-4H-benzo[6,7]-cyclo-hepta[1,2-b]thiophene-3-carboxamide,
2-(acetylamino)-8-methoxy-5,6-dihydro-4H-benzo[6,7]-cyclo-hepta[1,2-b]thiophene-3-carboxamide,
2-(acetylamino)-8-methoxy-6,6-dimethyl-5,6-dihydro-4H-benzo[6,7]-cyclo-hepta[
1,2-b]-thiophene-3-carboxamide,
2-(acetylamino)-9-methoxy-5,6-dihydro-4H-benzo[6,7]-cyclo-hepta[1,2-b]thiophene-3-carboxamide,
2-(acetylamino)-8,10-dimethoxy-5,6-dihydro-4H-benzo[6,7]-cyclo-hepta[1,2-b]thiophene-3-carboxamide,
2-(acetylamino)-8-methyl-5,6-dihydro-4H-benzo-[6,7]-cyclo-hepta[1,2-b]thiophene-3-carboxamide,
2-(acetylamino)-8,9-dimethoxy-5,6-dihydro-4H-benzo[6,7]-cyclo-hepta[1,2-b]thiophene-3-carboxamide,
2-(acetylamino)-8-fluoro-9-methoxy-5,6-dihydro-4H-benzo[6,7]-cyclo-hepta[
1,2-b]-thiophene-3-carboxamide,
2-(acetylamino)-7-methoxy-5,6-dihydro-4H-benzo[6,7]-cyclo-hepta[1,2-b]thiophene-3-carboxamide,
2-(acetylamino)-6,9-dimethyl-5,6-dihydro-4H-benzo[6,7]-cyclo-hepta[1,2-b]thiophene-3-carboxamide,
2-(acetylamino)-10-methoxy-5,6-dihydro-4H-benzo[6,7]-cyclo-hepta[1,2-b]thiophene-3-carboxamide,
2-(acetylamino)-9-bromo-6-methyl-5,6-dihydro-4H-benzo[6,7]-cyclo-hepta[1,2-b]thiophene-3-carboxamide,
2-(acetylamino)-7,8-dimethoxy-5,6-dihydro-4H-benzo[6,7]-cyclo-hepta[1,2-b]thiophene-3-carboxamide,
2-(acetylamino)-6-methyl-5,6-dihydro-4H-benzo-[6,7]cyclohepta-[1,2-b]thiophene-3-carboxamide,
2-(acetylamino)-9-chloro-6-methyl-5,6-dihydro-4H-benzo[6,7]-cyclo-hepta[
1,2-b]-thiophene-3-carboxamide,
2-(acetylamino)-9-fluoro-5,6-dihydro-4H-benzo-[6,7]-cyclo-hepta[1,2-b]thiophene-3-carboxamide,
2-(acetylamino)-9-methyl-5,6-dihydro-4H-benzo[6,7]-cyclohepta[1,2-b]thiophene-3-carboxamide,
2-(acetylamino)-9-amino-5,6-dihydro-4H-benzo[6,7]-cyclo-hepta[1,2-b]thiophene-3-carboxamide,
2-(acetylamino)-9-nitro-5,6-dihydro-4H-benzo[6,7]cycloheptata[1,2-b]thiophene-3-carboxamide,
2-(acetylamino)-9,10-dimethoxy-5,6-dihydro-4H-benzo[6,7]-cyclo-hepta[1,2-b]thiophene-3-carboxamide,
2-[(aminocarbonyl)amino]-5,6-dihydro-4H-benzo[6,7]-cyclohepta[1,2-b]thiophene-3-carboxamide,
2-[(aminocarbonyl)amino]-9-chloro-5,6-dihydro-4H-benzo[6,7]-cyclo-hepta[
1,2-b]thiophene-3-carboxamide,
2-[(aminocarbonyl)amino]-8,9-dichloro-5,6-dihydro-4H-benzo[6,7]-cyclo-hepta[
1,2-b]thiophene-3-carboxamide,
2-[(aminocarbonyl)amino]-8-methoxy-5,6-dihydro-4H-benzo[6,7]-cyclo-hepta[
1,2-b]thiophene-3-carboxamide,
2-[(aminocarbonyl)amino]-8-methoxy-6,6-dimethyl-5,6-dihydro-4H-benzo[6,7]-cyclo-hepta[
1,2-b]thiophene-3-carboxamide,
2-[(aminocarbonyl)amino]-9-methoxy-5,6-dihydro-4H-benzo[6,7]-cyclo-hepta[
1,2-b]thiophene-3-carboxamide,
2-[(aminocarbonyl)amino]-8,10-dimethoxy-5,6-dihydro-4H-benzo[6,7]-cyclo-hepta[
1,2-b]thiophene-3-carboxamide,
2-[(aminocarbonyl)amino]-8-methyl-5,6-dihydro-4H-benzo[6,7]10 cyclohepta[1,2-b]thiophene-3-carboxamide,
2-[(aminocarbonyl)amino]-8,9-dimethoxy-5,6-dihydro-4H-benzo[6,7]-cyclo-hepta[
1,2-b]thiophene-3-carboxamide,
2-[(aminocarbonyl)amino]-8-fluoro-9-methoxy-5,6-dihydro-4H-benzo[6,7]-cyclo-hepta[
1,2-b]thiophene-3-carboxamide,
2-[(aminocarbonyl)amino]-7-methoxy-5,6-dihydro-4H-benzo[6,7]-cyclo-hepta[
1,2-b]thiophene-3-carboxamide,
2-[(aminocarbonyl)amino]-6,9-dimethyl-5,6-dihydro-4H-benzo[6,7]-cyclo-hepta[
1,2-b]thiophene-3-carboxamide,
2-[(aminocarbonyl)amino]-10-methoxy-5,6-dihydro-4H-benzo[6,7]-cyclo-20 hepta[1,2-b]thiophene-3-carboxamide,
2-[(aminocarbonyl)amino]-9-bromo-6-methyl-5,6-dihydro-4H-benzo[6,7]-cyclo-hepta[
1,2-b]thiophene-3-carboxamide,
2-[(aminocarbonyl)amino]-7,8-dimethoxy-5,6-dihydro-4H-benzo[6,7]-cyclo-hepta[
1,2-b]thiophene-3-carboxamide,
2-[(aminocarbonyl)amino]-6-methyl-5,6-dihydro-4H-benzo[6,7]-cyclo-hepta[
1,2-b]thiophene-3-carboxamide,
2-[(aminocarbonyl)amino]-9-chloro-6-methyl-5,6-dihydro-4H-benzo[6,7]-cyclo-hepta[
1,2-b]thiophene-3-carboxamide,
2-[(aminocarbonyl)amino]-9-fluoro-5,6-dihydro-4H-benzo[6,7]30 cyclohepta[1,2-b]thiophene-3-carboxamide,
2-[(aminocarbonyl)amino]-9-methyl-5,6-dihydro-4H-benzo[6,7]-cyclo-hepta[
1,2-b]thiophene-3-carboxamide,
9-amino-2-[(aminocarbonyl)amino]-5,6-dihydro-4H-benzo-[6,7]-cyclo-hepta[
1,2-b]thiophene-3-carboxamide,
2-[(aminocarbonyl)amino]-9-nitro-5,6-dihydro-4H-benzo[6,7]-cyclo-hepta[1,2-b]thiophene-3-carboxamide, and
2-[(aminocarbonyl)amino]-9,10-dimethoxy-5,6-dihydro-4H-benzo[6,7]-cyclo-hepta[1,2-b]thiophene-3-carboxamide.

13 A composition comprising the compound of claim 1, 2, 3, 11, or 12, and at least one pharmaceutically acceptable carrier.

14 A method of treating cancer, inflammation or an inflammation associated disorder in a subject, said method comprising administering to the subject having or susceptible to such cancer, inflammation or inflammation associated disorder, a therapeutically-effective amount of a compound of claim 1, 2, 3, 11, or 12.

15 The method of claim 14 for use in the treatment of cancer.

16 The method of claim 14 for use in the treatment of inflammation.

17 The method of claim 14 for use in the treatment of an inflammation-associated disorder.

18 The method of claim 17 wherein the inflammation-associated disorder is arthritis.

19 The method of claim 17 wherein the inflammation-associated disorder is pain

20 The method of claim 17 wherein the inflammation-associated disorder is fever.

21 The method of claim 17 wherein the inflammation-associated disorder is asthma.

22 The method of claim 17 wherein the inflammation-associated disorder is inflammatory bowel disease.
